(19) United States
(12) Patent Application Publication
(10) Pub. No.: US 2024/0239786 A1
(43) Pub. Date: Jul. 18, 2024

(54) COMPOSITIONS AND METHODS FOR THE TREATMENT AND PREVENTION OF CANCER

(71) Applicant: Yale University, New Haven, CT (US)

(72) Inventors: Z Ping Lin, New Haven, CT (US); Elena Ratner, New Haven, CT (US); Yong-Lian Zhu, New Haven, CT (US)

(21) Appl. No.: 18/555,112

(22) PCT Filed: Apr. 11, 2022
(86) PCT No.: PCT/US22/24240

§ 371 Date: Oct. 12, 2023

Related U.S. Application Data
(60) Provisional application No. 63/173,772, filed on Apr. 12, 2021.

(51) Int. Cl. C07D 417/14; A61K 31/4196; A61K 31/454; A61K 31/4545; A61K 31/502; A61P 35/00; C07D 249/12; C07D 401/04; C07D 401/14; C07D 409/14

(52) U.S. Cl. CPC C07D 417/14; A61K 31/4196; A61K 31/454; A61K 31/4545; A61K 31/502; A61P 35/00; C07D 249/12; C07D 401/04; C07D 401/14; C07D 409/14

(57) ABSTRACT

The present invention relates to compounds represented by Formulae (I) to (V), or a racemate, enantiomer, diastereomer, pharmaceutically acceptable salt, solvate, hydrate, or derivative thereof, and compositions thereof. In various aspects, the present invention also relates to methods of reducing or inhibiting ribonucleotide reductase (RNR), poly ADP-ribose polymerase (PARP), and/or homologous recombination (HR) repair using said compounds. In some aspects, the present invention relates to methods of inducing at least one double strand break (DSB) using said compounds. In another aspect, the present invention relates to methods of treating or preventing cancer (e.g., ovarian cancer, BRCA-wild type cancer, etc.) using said compounds.

### CROSS-REFERENCE TO RELATED APPLICATIONS

This application claims priority to and the benefit of U.S. Provisional Application No. 63/173,772, filed Apr. 12, 2021, the disclosure of which is hereby incorporated by reference herein in its entirety.

### BACKGROUND OF THE INVENTION

Clinical approvals for poly(adenosine diphosphate(ADP)-ribose) polymerase (PARP) inhibitors provide new treatment options for women with epithelial ovarian cancer (EOC). Olaparib is the first-in-class PARP inhibitor approved for the treatment of patients with advanced breast cancer (BRCA)-mutated epithelial ovarian cancer (EOC) who have been treated with three or more prior lines of chemotherapy. Thereafter, olaparib has been approved for the first-line maintenance treatment of patients with recurrent platinum-sensitive EOC regardless of BRCA status. Two more PARP inhibitors rucaparib and niraparib have been subsequently approved for similar indications in EOC.

PARP inhibitors demonstrate clinical efficacy by targeting BRCA mutations or defects in homologous recombination (HR) repair in breast and ovarian cancers (Ratner E S et al., 2012, Curr Opin Oncol, 24:564-571; Audeh M W et al., 2010, Lancet, 376:245-251). PARP is a nuclear protein essential for the repair of DNA single strand breaks (SSBs) (Schreiber V et al., 2006, Nat Rev Mol Cell Biol, 7:517-528). PARP binds to SSBs and catalyzes polymerization of ADP-ribose chains which signal and recruit other proteins to engage the repair process (El-Khamisy S F et al., 2003, Nucleic Acids Res, 31:5526-5533; Dantzer F et al., 1999, Biochimie, 81:69-75).

It has been suggested that inhibition of PARP leads to persistent SSBs (Trucco C et al., 1998, Nucleic Acids Res, 26:2644-2649; Pachkowski B F et al., 2009, Mutat Res, 671:93-99), which are converted into DNA double strand breaks (DSBs) by replication forks (Kuzminov A et al., 2001, Proc Natl Acad Sci USA, 98:8241-8246). Thus, the resulting DSBs are necessarily repaired by HR (Arnaudeau C et al., 2001, J Mol Biol, 307:1235-1245; Woodhouse B C et al., 2008, DNA Repair (Amst), 7:932-940). It has been alternatively posited that HR repair is critical for replication restart or bypass of stalled replication forks in PARP-trapped SSB intermediates (Helleday T et al., 2011, Mol Oncol, 5:387-393). Breast cancer gene 1 (BRCA1) and breast cancer gene 2 (BRCA2) proteins are critical components of the HR pathway. Therefore, cancer cells with BRCA1 and BRCA2 mutations or defects in the HR pathway are hypersensitive to the lethality of PARP inhibitors (Bryant H E et al., 2005, Nature, 434:913-917; Farmer H et al., 2005, Nature 434:917-921).

However, the effectiveness of PARP inhibitors is limited to EOC with BRCA mutations (˜15%) (Jayson G C et al., 2014, Lancet, 384:1376-1388) and about 50% of high-grade serous EOC that exhibits HR deficiency (Turner N et al., 2004, Nat Rev Cancer, 4:814-819). A significant portion of EOC remains resistant to PARP inhibitors. Furthermore, the reversion of mutated BRCA genes to restore HR repair function have been identified in both preclinical and clinical studies of EOC with acquired resistance to platinum and PARP inhibitors (Sakai W et al., 2008, Nature, 451:1116-1120; Swisher E M et al., 2008, Cancer Res, 68:2581-2586; Norquist B et al., 2011, J Clin Oncol, 29:3008-3015). Given the growing use of PARP inhibitors, the cases of patients with PARP inhibitor resistant EOC is predictably on the rise.

Thus, there is a need in the art for compounds and methods for inhibiting poly ADP-ribose polymerase (PARP), ribonucleotide reductase (RNR), homologous recombination (HR) repair, or any combination thereof and treating or preventing cancers, such as cancers that are resistant to PARP inhibitors. The present invention addresses this unmet need in the art.

### SUMMARY OF THE INVENTION

In one aspect, the present invention relates, in part, to a compound represented by one of Formulae (I) to (V):

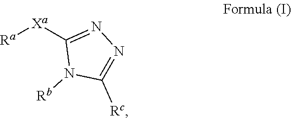

or a racemate, enantiomer, diastereomer, pharmaceutically acceptable salt, solvate, hydrate, or derivative thereof;

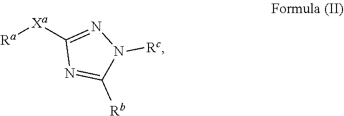

or a racemate, enantiomer, diastereomer, pharmaceutically acceptable salt, solvate, hydrate, or derivative thereof;

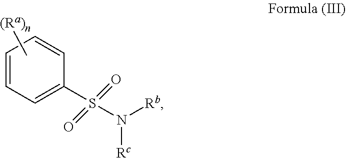

or a racemate, enantiomer, diastereomer, pharmaceutically acceptable salt, solvate, hydrate, or derivative thereof;

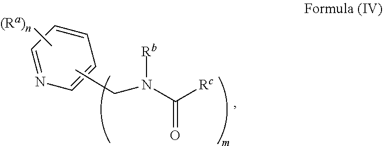

or a racemate, enantiomer, diastereomer, pharmaceutically acceptable salt, solvate, hydrate, or derivative thereof; or

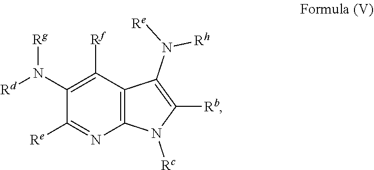

or a racemate, enantiomer, diastereomer, pharmaceutically acceptable salt, solvate, hydrate, or derivative thereof.

In some embodiments, each occurrence of X^{a} is independently selected from O, S, —SO₂, —N(R^{x}), —C(R^{x})(R^{y}), or —C═R^{z}.

In some embodiments, each occurrence of R^{a}, R^{b}, R^{c}, R^{d}, R^{e}, R^{f}, R^{g}, R^{h}, R^{x}, and R^{y} is independently selected from hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, or any combination thereof. In some embodiments, each occurrence of R^{a}, R^{b}, and R^{c} is independently selected from hydrogen, deuterium, fluoro, chloro, bromo, iodo, alkyl, aryl, aryl alkyl, heteroaryl, heteroaryl alkyl, alkoxycarbonyl, amino, aminoalkyl, aminoalkyl-aryl, aminoalkyl-heteroaryl, amido, secondary amide, tertiary amide, aminoalkenyl, aminoalkynyl, aminoacetate, acyl, hydroxyl, hydroxyalkyl, hydroxyaryl, hydroxyalkyl-aryl, hydroxyheteroaryl, hydroxyalkyl-heteroaryl, alkoxy, carboxyl, carboxylate, ester, aminoaryl, aminoaryl alkyl, aminoheteroaryl, aminoheteroaryl alkyl, thioaryl, thioaryl alkyl, thioheteroaryl, thioheteroaryl alkyl, or any combination thereof.

In some embodiments, R^{z} is selected from O, S, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, or any combination thereof.

In some embodiments, R^{a}, R^{b}, R^{c}, R^{d}, R^{e}, R^{f}, R^{g}, R^{h}, R^{x}, R^{y}, and R^{z} are each independently optionally substituted.

In some embodiments, R^{b} and R^{c} are optionally joint to form a cycle.

In some embodiments, m is an integer represented by 0 to 5. In one embodiment, m is an integer represented by 1.

In some embodiments, n is an integer represented by 0 to 5.

In some embodiments, R^{a}—X^{a} is

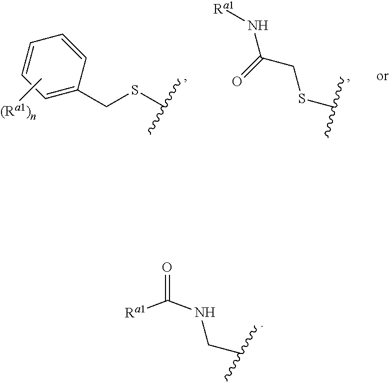

In some embodiments, R^{b} is

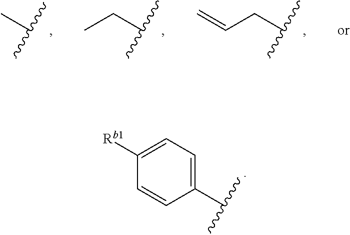

In some embodiments, R^{c} is

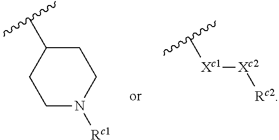

In some embodiments, the compound represented by Formula (I) is selected from

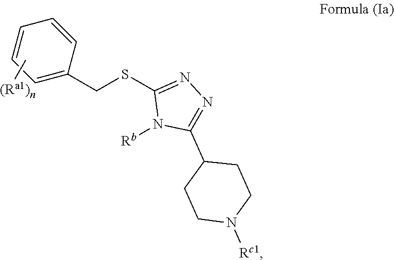

or a racemate, enantiomer, diastereomer, pharmaceutically acceptable salt, solvate, hydrate, or derivative thereof;

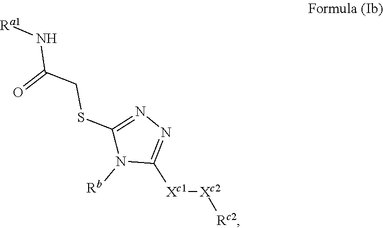

or a racemate, enantiomer, diastereomer, pharmaceutically acceptable salt, solvate, hydrate, or derivative thereof; and

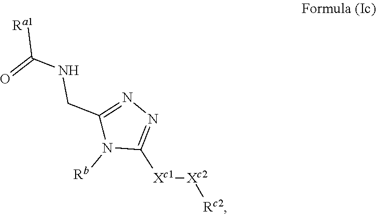

or a racemate, enantiomer, diastereomer, pharmaceutically acceptable salt, solvate, hydrate, or derivative thereof.

In some embodiments, each occurrence of R^{a1} is independently selected from hydrogen, deuterium, fluoro, chloro, bromo, iodo, alkyl, aryl, aryl alkyl, heteroaryl, heteroaryl alkyl, aminoaryl, aminoaryl alkyl, aminoheteroaryl, aminoheteroaryl alkyl, thioaryl, thioaryl alkyl, thioheteroaryl, thioheteroaryl alkyl, or any combination thereof.

In some embodiments, R^{b1} is selected from hydrogen, deuterium, fluoro, chloro, bromo, iodo, alkyl, or any combination thereof.

In some embodiments, R^{c1} and R^{c2} are independently selected from hydrogen, deuterium, fluoro, chloro, bromo, iodo, alkyl, aryl, aryl alkyl, heteroaryl, heteroaryl alkyl, alkoxycarbonyl, acyl, hydroxyl, hydroxyalkyl, hydroxyaryl, hydroxyalkyl-aryl, hydroxyheteroaryl, hydroxyalkyl-heteroaryl, alkoxy, carboxyl, carboxylate, ester, aminoaryl, aminoaryl alkyl, aminoheteroaryl, aminoheteroaryl alkyl, thioaryl, thioaryl alkyl, thioheteroaryl, thioheteroaryl alkyl, or any combination thereof. In some

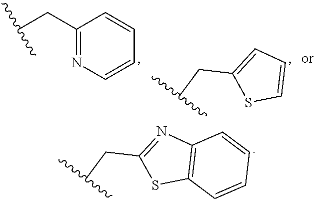

embodiments, R^{c1} is hydrogen, methyl, ethyl,

In some embodiments, X^{c1} and X^{c2} are independently selected from O, S, —SO₂, —N(R^{x}), or —C(R^{x})(R^{y}).

In some embodiments, R^{x} and R^{y} are independently selected from hydrogen or alkyl.

In some embodiments, R^{a1}, R^{b1}, R^{c1}, R^{c2}, R^{x}, and R^{y} are each independently optionally substituted.

In some embodiments, the compound represented by Formula (Ia) is selected from

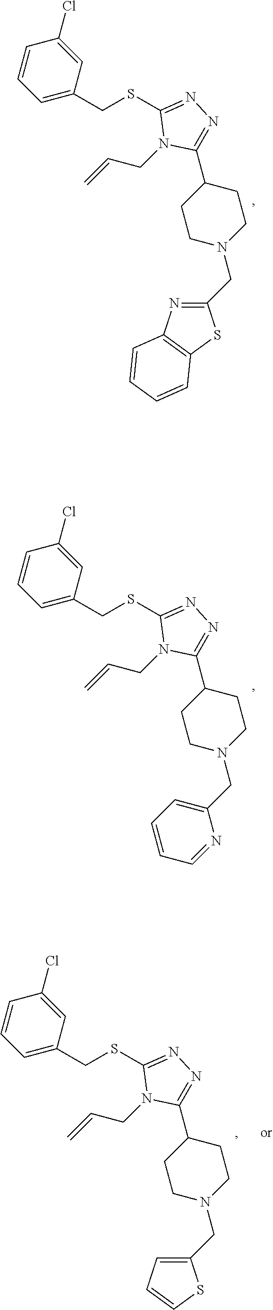 [SMILES: C=CCn1c(SCc2cccc(Cl)c2)nnc1C1CCN(Cc2nc3ccccc3s2)CC1 ; C=CCn1c(SCc2cccc(Cl)c2)nnc1C1CCN(Cc2ccccn2)CC1 ; C=CCn1c(SCc2cccc(Cl)c2)nnc1C1CCN(Cc2cccs2)CC1]

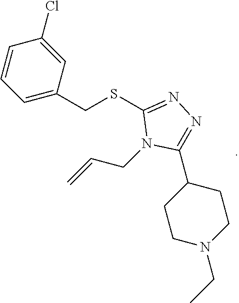 [SMILES: C=CCn1c(SCc2cccc(Cl)c2)nnc1C1CCN(CC)CC1]

In some embodiments, the compound represented by Formula (Ib) is selected from

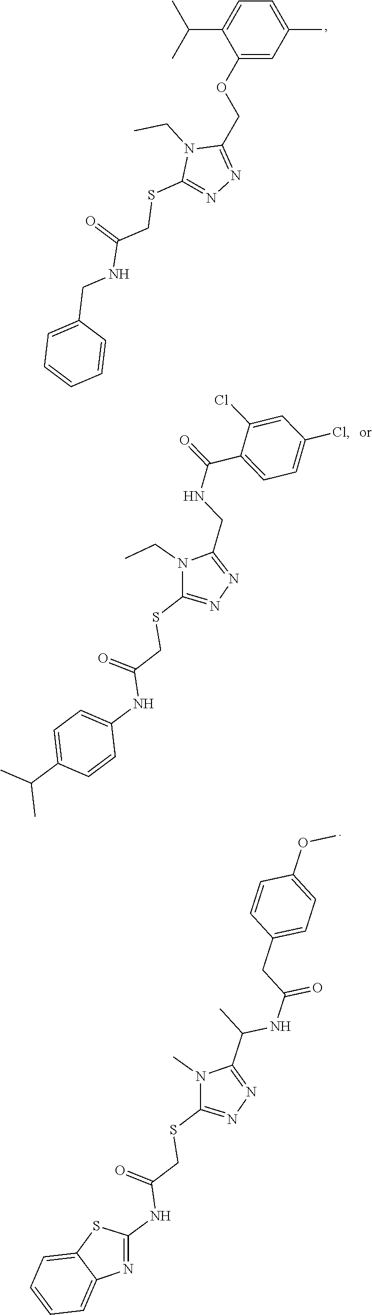

In one embodiment, the compound represented by Formula (Ic) is

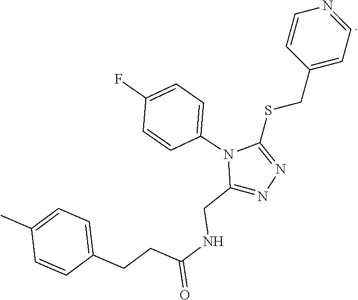 [SMILES: Cc1ccc(CCC(=O)NCc2nnc(SCc3ccncc3)n2-c2ccc(F)cc2)cc1]

In some embodiments, the compound represented by Formula (II) is selected from

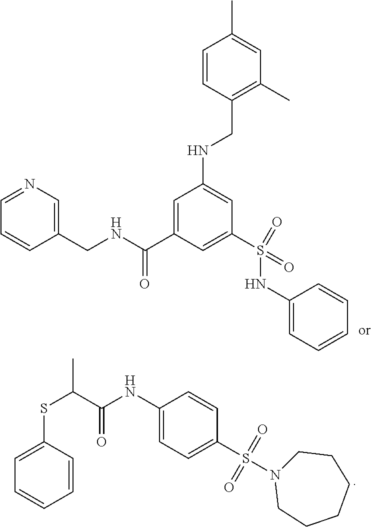 [SMILES: Cc1ccc(CNc2cc(C(=O)NCc3cccnc3)cc(S(=O)(=O)Nc3ccccc3)c2)c(C)c1 ; CC(Sc1ccccc1)C(=O)Nc1ccc(S(=O)(=O)N2CCCCCC2)cc1]

In some embodiments, the compound is a ribonucleotide reductase (RNR) inhibitor.

In some embodiments, the compound represented by Formula (IV) is a compound represented by Formula (IVa)

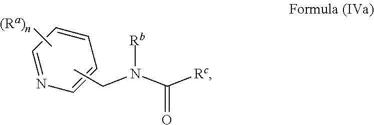

or a racemate, enantiomer, diastereomer, pharmaceutically acceptable salt, solvate, hydrate, or derivative thereof.

In one aspect, the present invention relates, in part, to a composition comprising at least one compound of the present invention. In some embodiments, the composition further comprises at least one poly ADP-ribose polymerase (PARP) inhibitor. In some embodiments, the poly ADP-ribose polymerase (PARP) inhibitor is olaparib.

In one aspect, the present invention relates, in part, to a method of reducing the level or activity of poly ADP-ribose polymerase (PARP). In another aspect, the present invention relates, in part, to a method of reducing the level or activity of ribonucleotide reductase (RNR). In another aspect, the present invention relates, in part, to a method of reducing homologous recombination (HR) repair. In another aspect, the present invention relates, in part, to a method of inducing at least one double strand break (DSB). In another aspect, the present invention relates, in part, to a method of reducing the level or activity of cyclin A2. In another aspect, the present invention relates, in part, to a method of reducing the level or activity of Rad51 foci.

In one aspect, the present invention relates, in part, to a method of regulating the level or activity of at least one selected from the group consisting of checkpoint kinase 1 (Chk1), ataxia telangiectasia and Rad3-related protein (ATR), replication protein A32 (RPA32), histone H2A, such as H2AX, histone H1, cyclin-dependent kinase 1 (CDK1), and cyclin-dependent kinase 2 (CDK2). In another aspect, the present invention relates, in part, to a method of regulating the phosphorylation of at least one selected from the group consisting of checkpoint kinase 1 (Chk1), ataxia telangiectasia and Rad3-related protein (ATR), replication protein A32 (RPA32), histone H2A, such as H2AX, histone H1, cyclin-dependent kinase 1 (CDK1), and cyclin-dependent kinase 2 (CDK2).

In one aspect, the present invention relates, in part, to a method of increasing the level or activity of programmed death-ligand 1 (PD-L1). In another aspect, the present invention relates, in part, to a method of increasing the level or activity of at least one tumor neoantigen. In various embodiments, the method comprises administering a therapeutically effective amount of at least one compound of the present invention or a composition thereof.

In one aspect, the present invention relates, in part, to a method of preventing or treating cancer in a subject in need thereof. In another aspect, the present invention relates, in part, to a method of administering at least one compound of claim 1 or a composition thereof to a subject. In various embodiments, the method comprises administering a therapeutically effective amount of at least one compound of the present invention or a composition thereof to the subject. In one embodiment, the cancer is ovarian cancer, BRCA-wild type cancer, or a combination thereof.

In some embodiments, the method further comprises administering at least one poly ADP-ribose polymerase (PARP) inhibitor. In one embodiment, the at least one PARP inhibitor is olaparib. In some embodiments, the at least one PARP inhibitor is administered at the same time as the compound of claim 1 or a composition thereof.

### BRIEF DESCRIPTION OF THE DRAWINGS

The following detailed description of preferred embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A through FIG. 1D, depicts representative results for in silico screening and hit clustering for small molecule inhibitors of ribonucleotide reductase (RNR). FIG. 1A depicts a schematic representation of surface rendering for the triapine-binding pocket of the R2 subunit of RNR. A docking pose of triapine in the surface cavity of the putative triapine-binding pocket was modeled by the GOLD docking program and visualized using the PyMOL program. FIG. 1B depicts a schematic diagram of molecular interactions between triapine and the triapine-binding pocket. The docking pose of triapine shown in FIG. 1A was run by the LigPlot+ program to generate the 2D representation of two hydrogen bonds and eight hydrophobic interactions with the binding pocket. FIG. 1C depicts representative results demonstrating the hit clustering of top-ranked 200 compounds. These in silico hits were clustered to 20 groups. The percentage of hits of each group and the average of GOLDScore within each group are shown. FIG. 1D depicts schematic representation of structures of top 5 pharmacophore groups A, B, DB, G, and M. R₁, R₂, and R₃ represent sides chain with varying structure in each group. Each group consists of 7-15% of top-ranked 200 compounds.

FIG. 2, comprising FIG. 2A through FIG. 2F, depicts representative results of follow-up assays of in silico hit compounds in PEO1 and PEO4 cells. FIG. 2A depicts representative results of cytotoxicity assays on selected 25 in silico hit compounds in PEO1 and PEO4 cells. Cells were plated for 24 hr and then treated with 50 μM compounds for 72 hr. For a positive control, cells were treated with 1 μM triapine for 72 hr. The percentage of cell survival was determined by the MTS cytotoxicity assay. FIG. 2B depicts representative results demonstrating that DNA synthesis assays on 10 compounds caused a 25% or more decrease in cell survival from the MTS assay. Cells were treated with 50 μM compounds or 1 μM triapine for 24 hr. During the last hour, cells were pulse-labeled with EdU, stained with the Click-iT EdU Alexa Fluor 488 Assay Kit, counterstained with 7-AAD, and subsequently analyzed by flow cytometry. EdU-positive cells were gated to determine the percentage of the S phase cell population. The bivariate plots of Alexa Fluor 488 (EdU-positive) and PE-Cy5 (7-AAD-positive) are shown. FIG. 2C depicts representative results demonstrating that DNA synthesis assays on 10 compounds caused a 25% or more decrease in cell survival from the MTS assay. Cells were treated with 50 μM compounds or 1 μM triapine for 24 hr. During the last hour, cells were pulse-labeled with EdU, stained with the Click-iT EdU Alexa Fluor 488 Assay Kit, counterstained with 7-AAD, and subsequently analyzed by flow cytometry. EdU-positive cells were gated to determine the percentage of the S phase cell population. The percentages of DNA synthesis in cells treated with each of 10 compounds and triapine relative to the DMSO-treated control is calculated and shown. FIG. 2D depicts representative results of dNTP measurement on triapine and DB4 in PEO4 cells. Cells were treated with 1 μM triapine or 50 μM DB4 for 24 hr. Cells were extracted with 65% methanol for determination of each of dNTPs expressed as pmole/10⁶ cells. Data are means±SD (N=3). *, p<0.05; **, p<0.01; ***, p<0.001, compared with the DMSO-treated control in each dNTP. FIG. 2E depicts representative results demonstrating reversal of RNR inhibition by supplementing deoxyribonucleosides (dNs). PEO1 cells were pre-treated with 1 mM dA plus 100 mM dG, 10 mM dC plus 10 mM dT, or all dNs for 1 hr and then treated with various concentrations of DB4 or triapine for 72 hr. The percentage of cell survival was determined by the MTS cytotoxicity assay. Data are means±SD (N=3). *, p<0.05; **, p<0.01; ***, p<0.001, compared with the control in each concentration of DB4 or triapine. FIG. 2F depicts schematic representation of structures and molecular compound names of triapine, DB4, and A4.

FIG. 3, comprising FIG. 3A through FIG. 3E, depicts representative results demonstrating that structural activity relationship (SAR) of DB4 analogs and molecular modeling of DB4 bound to the R2 subunit of RNR. FIG. 3A depicts schematic representation of structures of DB4 and its analogs, DB4-A, DB4-C, and DB4-F. FIG. 3B depicts representative results demonstrating effects of DB4 analogs on DNA synthesis. PEO1 and PEO4 cells were treated with 50 μM DB4 or analogs and assayed for DNA synthesis inhibition as described in FIG. 2B. The bivariate plots of Alexa Fluor 488 (EdU-positive) and PE-Cy5 (7-AAD-positive) are shown in FIG. 4. The percentage of S phase cells treated with DB4 or DB4 analogs relative to the DMSO-treated control was calculated and shown. FIG. 3C depicts a schematic representation of the surface rendering of the triapine-binding pocket and a putative docking pose of DB4. Only residues putatively interacting with DB4 are displayed in surface rendering.

FIG. 3D depicts representative schematic diagram of molecular interactions between DB4 and the triapine-binding pocket. The docking pose of DB4 shown in FIG. 3C was run by the LigPlot+ program to generate the 2D representation of one hydrogen bond and fifteen hydrophobic interactions with the binding pocket. FIG. 3E depicts representative results demonstrating effects of in silico mutagenesis of the triapine-binding pocket on the docking scores of DB4. Sixteen key amino acid residues were mutated to alanine using the PyMOL program. DB4 was re-docked into each of mutated triapine-binding pockets using the GOLD program. The GOLDScores of top-ranking docking poses similar to the wild-type (WT) control were averaged. Data are means±SD (N=3). *, p<0.05 compared with the WT control. ND, no similar docking poses detected.

FIG. 4 depicts representative results demonstrating effects of DB4 and its analogs on DNA synthesis. Cells were treated with 50 μM compounds for 24 hr. During the last hour, cells were pulse-labeled with EdU, stained with the Click-iT EdU Alexa Fluor 488 Assay Kit, counterstained with 7-AAD, and subsequently analyzed by flow cytometry. EdU-positive cells were gated to determine the percentage of the S phase cell population. The bivariate plots of Alexa Fluor 488 (EdU-positive) and PE-Cy5 (7-AAD-positive) are shown.

FIG. 5, comprising FIG. 5A through FIG. 5D, depicts representative results demonstrating chemical stability of DB4. DB4 remained mostly active and intact after 72 hr in the culture medium. The dose response curve of DB4 in DNA synthesis inhibition in PEO4 cells was generated as determined by EdU flow cytometric analysis. FIG. 5A depicts representative results demonstrating that DB4 was added to the cell culture medium at 30 μM and incubated for 0 hr, 24 hr, 48 hr, or 72 hr at 37° C. PEO4 cells were treated with these media for 24 hr. The level of DNA synthesis inhibition was determined to interpolate and calculate % DB4 remaining compared with the control at 0 hr. FIG. 5B depicts representative results for remaining DB4 after DB4 (MW=496.1) was added to the culture medium at 50 μM for 0 hr, 24 hr, 48 hr, or 72 hr at 37° C. FIG. 5C depicts representative mass spectrum obtained from mass spectrometry that was run to detect the level of the 496.13 ion at 0 hr. FIG. 5D depicts representative mass spectrum obtained from mass spectrometry that was run to detect the level of the 496.13 ion at 72 hr.

FIG. 6, comprising FIG. 6A through FIG. 6F, depicts representative results demonstrating the time course of inhibition of DNA synthesis and CDK2 activity by DB4 or triapine. FIG. 6A depicts representative results demonstrating time-dependent DNA synthesis inhibition by DB4 or triapine. PEO1 cells were treated with 30 μM DB4 or 1 μM triapine for indicated times, pulsed with EdU for 1 hr and subsequently assayed for DNA synthesis inhibition as described in FIG. 2B. The bivariate plots of Alexa Fluor 488 and PE-Cy5 were gated to determine % S phase cells. FIG. 6B depicts representative results demonstrating time-dependent DNA synthesis inhibition by DB4 or triapine. PEO4 cells were treated with 30 μM DB4 or 1 μM triapine for indicated times, pulsed with EdU for 1 hr and subsequently assayed for DNA synthesis inhibition as described in FIG. 2B. The bivariate plots of Alexa Fluor 488 and PE-Cy5 were gated to determine % S phase cells. FIG. 6C depicts representative results demonstrating changes in cell cycle distribution caused by DB4 or triapine. The bivariate plots of Alexa Fluor 488 and PE-Cy5 shown in FIG. 6A and FIG. 6B were gated to determine % cells in G1, S, and G2/M phases. FIG. 6D depicts representative results demonstrating time-dependent inhibition of CDK2 activity by DB4 or triapine. PEO4 cells were treated with 30 μM DB4 or 1 μM triapine for various time periods and subjected to western blot analysis for the levels of cyclin A2, p-CDK1/2, p-H1, p-Chk1, and HSC70. The HSC70 protein level was used as a loading control. Cropped gel images of protein bands are presented. Full-length gel images are shown in FIG. 14A. Cyclin A2, p-CDK1/2, and p-H1 bands were from re-probing the same blot. p-Chk1 and HSC70 bands were from re-probing the same duplicated blot. FIG. 6E depicts representative results demonstrating time-dependent inhibition of CDK2 activity by DB4 or triapine. The CDK2 activity is expressed by the percentage of the p-HI levels relative to that of the 0 hr control is shown. FIG. 6F depicts representative results demonstrating the effects of DB4 and triapine on double strand break (DSB) end resection. PEO4 cells were pre-treated with 30 μM DB4 or 1 μM triapine for 1 hr and then treated with 5 μM etoposide for 4 hr. Protein was analyzed by western blotting for p-Chk1, p-ATR, p-RPA32, γH2AX, and HSC70. Cropped gel images of protein bands are presented. Full-length gel images are shown in FIG. 14B. All protein bands were from re-probing the same blot. DSB end resection is assessed by the level of p-RPA32. The level of γH2AX was used to confirm DSBs induced by etoposide.

FIG. 7, comprising FIG. 7A through FIG. 7F, depicts representative results demonstrating that DB4 suppressed homologous recombination (HR) repair in ovarian cancer (BRCA)-wild type SKOV3 cells. FIG. 7A depicts representative results demonstrating effects of DB4 on HR activity in SKOV3-DR-GFP cells. Cells were transfected with the ISceI-expressing plasmid and then treated various concentrations of DB4 for 48 hr. Cells were harvested, stained with 7-AAD, and analyzed by flow cytometry for GFP-positive cells indicative of HR repair activity. FIG. 7B depicts representative results demonstrating effects of DB4 on NHEJ activity in SKOV3-EJ5-GFP cells. Cells were transfected with the ISceI-expressing plasmid and treated with various concentrations of DB4. Cells were harvested, stained with 7-AAD, and stained, and analyzed by flow cytometry for GFP-positive cells indicative of NHEJ activity. Data are means±SD (N=3). *, p<0.05; **, p<0.01, compared with the 0 μM control of ISceI-transected cells. FIG. 7C depicts representative results of confocal imaging of olaparib-induced nuclear Rad51 foci in SKOV3 cells treated DB4. Cells were treated with 30 μM DB4 for 1 hr and then treated with 25 μM olaparib or 6 hr. Cells were fixed, permeabilized, and stained with the anti-Rad51 antibody and the Alexa Fluor 488-conjugated secondary antibody. Nuclei were counterstained with DAPI. Immunofluorescence of Rad51 foci and nuclei were visualized by confocal microscopy. FIG. 7D depicts representative results demonstrating that DB4 significantly attenuated olaparib-induced Rad51 foci in SKOV3 cells. Cells were also scored for nuclei containing equal or more than 10 distinct Rad51 foci to determine the percentage of cells positive for Rad51 foci. Data are means±SD (N=10). p<0.001, compared between indicated treatment groups. FIG. 7E depicts representative results demonstrating effects of DB4 on olaparib-induced γH2AX in PEO1 and PEO4 cells. Cells were pre-treated with 30 μM DB4 and then treated with 25 μM olaparib for 24 hr. Protein was assessed by western blot analysis for the levels of γH2AX, p-H1, cyclin A2, p-CDK1/2, and HSC70. Cropped gel images of protein bands are presented. Full-length gel images are shown in FIG. 15. p-H1, cyclin A2, p-CDK1/2, and HSC70 bands were from re-probing the same blot. The γH2AX band was from a duplicated blot. FIG. 7F depicts representative results demonstrating effects of DB4 on olaparib-induced γH2AX in PEO1 and PEO4 cells. Cells were pre-treated with 30 μM DB4 and then treated with 25 μM olaparib for 24 hr. The band density of γH2AX indicative of DSBs was quantified by densitometry. Data are means±SD (N=3). p<0.05, compared between indicated treatment groups.

FIG. 8, comprising FIG. 8A through FIG. 8J, depicts representative results demonstrating that DB4 enhanced olaparib-induced DSBs and sensitized BRCA-wild EOC cells to olaparib. FIG. 8A depicts representative results demonstrating effects of DB4 on the sensitivities of PEO1 or PEO4 cells to olaparib. Cells were pre-treated with 30 μM DB4 for 1 hr and then treated with various concentrations of olaparib for 72 hr. Cell survival was determined by MTS cytotoxicity assay. Data are means±SD (N=3-4). FIG. 8B depicts representative results demonstrating that DB4 synergizes with olaparib to kill PEO4 cells. EOB calculation was used to quantify the synergy of the combination of DB4 and various concentrations of olaparib in PEO1 or PEO4 cells as shown in FIG. 8A. EOB>0, synergism; EOB=0, additivity; EOB<0, antagonism. FIG. 8C depicts representative results demonstrating effects of DB4F on the sensitivities of PEO1 or PEO4 cells to olaparib. Cells were pre-treated with 30 μM DB4-F for 1 hr and then treated with various concentrations of olaparib for 72 hr. Cell survival was determined by MTS cytotoxicity assay. Data are means±SD (N=3-4). FIG. 8D depicts representative results demonstrating that DB4 synergizes with olaparib to kill PEO4 cells. EOB calculation was used to quantify the synergy of the combination of DB4-F and various concentrations of olaparib in PEO1 or PEO4 cells as shown in FIG. 8B. EOB>0, synergism; EOB=0, additivity; EOB<0, antagonism. FIG. 8E depicts representative results demonstrating that olaparib-resistant PEO1 (PEO1-OR) cells restored wild type BRCA2 expression by TAG to TTG reversion mutation at position 5193 nt. Olaparib-resistant PEO1 clones were examined by Sanger DNA sequencing and western blot analysis of BRCA2 expression compared with PEO1 and PEO4 cells. Cropped gel images of protein bands are presented. Full-length gel images are shown in FIG. 16. BRCA2 and HSC70 bands were from re-probing the same blot. FIG. 8F depicts representative results demonstrating effects of various concentrations of DB4 on the sensitivity of PEO1-OR cells to olaparib. Cells were pre-treated with 0 μM, 5 μM, 10 μM, or 20 μM DB4 for 1 hr and then treated with various concentrations of olaparib for 72 hr. Cell survival was determined by MTS cytotoxicity assay. Data are means±SD (N=3-4). FIG. 8G depicts representative results demonstrating that DB4 and olaparib synergistically induces apoptosis in PEO4. Cells were pre-treated with various concentrations of DB4 for 1 hr and then treated with 50 μM olaparib for 24 hr. Caspase 3/7 activity was expressed as RLU per μg/μl protein. Data are means±SD (N=3). FIG. 8H depicts representative results demonstrating that DB4 and olaparib did not synergistically induce apoptosis in PEO1 cells. Cells were pre-treated with various concentrations of DB4 for 1 hr and then treated with 50 μM olaparib for 24 hr. Caspase 3/7 activity was expressed as RLU per μg/μl protein. Data are means±SD (N=3). FIG. 8I depicts representative results demonstrating that DB4 had no effects on olaparib-induced apoptosis in Hs 832(C).T cells. Cells were treated and caspase 3/7 activity was measured as described in FIG. 8G and FIG. 8H. FIG. 8J depicts representative results demonstrating that the fold change of olaparib-induced caspase 3/7 activity with increasing DB4 concentrations, shown for PEO1, PEO4, and Hs 832(C).T cells.

FIG. 9, comprising FIG. 9A and FIG. 9B, depicts representative results demonstrating that DB4 sensitized BRCA-wild type SKOV3 ovarian cancer and MDA-MB231 breast cancer cells to olaparib. Cells were plated for 24 hr, pre-treated with DB4 for 1 hr, and then treated with various concentrations of olaparib for 14 days. Colonies were stained and determined for clonogenic survival. FIG. 9A depicts representative results demonstrating that DB4 sensitized BRCA-wild type SKOV3 cells to olaparib. FIG. 9B depicts representative results demonstrating that DB4 sensitized BRCA-wild type MDA-MB231 cells to olaparib.

FIG. 10, comprising FIG. 10A through FIG. 10F, depicts representative results demonstrating that the combination of DB4 and olaparib suppressed the growth of BRCA-wild type EOC xenografts in mice. FIG. 10A depicts representative results demonstrating that the DB4-olaparib combination caused significant prolongation of the survival time of SCID-beige mice bearing with PEO4ip xenografts. SCID-Beige mice implanted ip with PEO4ip cells were treated daily (qd) with DB4 (10 mg/kg), olaparib (50 mg/kg), and the DB4-olaparib combination for 4 weeks. Kaplan Meier analysis was performed to determine the survival time of mice following treatments. Statistical significance was determined by the Mantel-Cox test comparing the control with each treatment group (N=4-5). FIG. 10B depicts representative results demonstrating that the DB4-olaparib combination used in FIG. 10A exhibited no obvert toxicity to SCID-beige mice as determined by the body weight. FIG. 10C depicts representative results demonstrating that the modified DB4-olaparib combination furthered significant prolongation of the survival time of SCID-beige mice bearing with PEO4ip xenografts. DB4 given every two days (q2d) in the DB4-olaparib combination for 4 or 6 weeks (n=4-5). FIG. 10D depicts representative results demonstrating that the DB4-olaparib combination used in FIG. 10C exhibited no obvert toxicity to SCID-beige mice as determined by the body weight. FIG. 10E depicts representative results demonstrating that the DB4-olaparib combination concertedly suppressed the sc growth of SKOV3 xenografts in mice. NOD CRISPR Prkdc Il2r Gamma (NCG) mice implanted sc with SKOV3 cells were treated as described in FIG. 10C for 4 weeks during which the tumor size was measured. Statistical significance was determined by the Wilcoxon matched-pairs signed test comparing the control with each treatment group and comparing between treatment groups. Data are means±SE (N=4-5). *, p<0.05; **, p<0.01. FIG. 10F depicts representative results demonstrating that the DB4-olaparib combination used in FIG. 10E exhibited no obvert toxicity to NCG mice as determined by the body weight.

FIG. 11 depicts representative predicted physical and chemical properties of A4 and DB4 analogs for evaluation of drug likeness. MW (molecular weight); c Log P (octanol/water partition coefficient; Log SW (intrinsic water solubility); RB (rotatable bond); tPSA (topological polar surface area); hDON (hydrogen bond donor); hACC (hydrogen bond acceptor).

FIG. 12 depicts schematic 3D representations of DB4 and DB4 analog docking poses in the triapine binding pocket. Yellow arrow indicates the protrusion of the benzene ring of the benzothiazole group of DB4 above the binding pocket.

FIG. 13, comprising FIG. 13A and FIG. 13B, depicts schematical representations of triapine and DB4 inhibiting CDK2 activity and HR repair. RFS, replication fork stalling; Bold solid line, strong; Solid line, medium; Dash line, weak.

FIG. 13A depicts schematic representation of triapine inhibiting CDK2 activity and HR repair. Triapine promptly and strongly inhibited CDK2 by ATR and Chk1, thereby blocking DSB end resection and HR repair predominately through routes 1 and 2 (in circle). FIG. 13B depicts schematic representation of DB4 inhibiting CDK2 activity and HR repair. DB4 gradually and weakly inhibited CDK2 and HR repair through route 2, allowing DSB end resection to occur (route 1). As a result, extensively resected DSB ends activated ATR and Chk1 to enforce the inhibition of CDK2 through route 3, leading to impairment of HR repair.

FIG. 14, comprising FIG. 14A and FIG. 14B, depicts representative full length gel images of FIG. 6D and FIG. 6F. Dash lines indicate the border of blots.

FIG. 14A depicts representative full length gel images of FIG. 6D. FIG. 14B depicts representative full length gel images of FIG. 6F.

FIG. 15 depicts representative full length gel images of FIG. 7E. Dash lines indicate the border of blots.

FIG. 16 depicts representative full length gel images of FIG. 8E. Dash lines indicate the border of blots.

FIG. 17 depicts representative comparison of exemplary in vitro and in vivo dosages between DB4 and other clinically used and FDA-approved RNR and PARP inhibitors.

FIG. 18, comprising FIG. 18A through FIG. 18C, depicts representative preliminary SAR for exemplary in silico hits of experimentally active RNR inhibitors.

FIG. 18A depicts exemplary in silico lead compounds of experimentally active RNR inhibitors. FIG. 18B depicts exemplary top in silico hits of experimentally active RNR inhibitors. FIG. 18C depicts representative pharmacophore overlap of exemplary top in silico hits of experimentally active RNR inhibitors.

FIG. 19 depicts schematical representation of DB4 increasing programmed death-ligand 1 (PD-L1) for harnessing an immune checkpoint inhibitor to treat BRCA-wild type EOC.

FIG. 20, comprising FIG. 20A and FIG. 20B, depicts representative effects of olaparib and/or IFNγ on BRCA-mutated PEO1 and BRCA-wild type PEO4 cells.

FIG. 20A depicts representative results demonstrating that BRCA-mutated PEO1 cells exhibited an increase in basal and olaparib-induced PD-L1 compared with BRCA-wild type PEO4 cells. FIG. 20B depicts representative results demonstrating that IFNγ markedly augmented the levels of basal and olaparib-induced PD-L1 in PEO1 cells while having relatively minor effects on that of PEO4 cells.

FIG. 21, comprising FIG. 21A through FIG. 21C, depicts representative results demonstrating that DB4 and/or olaparib increased DSBs (γH2AX) and PD-L1 in BRCA-wild type PEO4 cells. FIG. 21A depicts representative effects of DB4 and/or olaparib on BRCA-wild type PEO4 cells after 24, 48, and 72 hr. FIG. 21B depicts representative effects of DB4 and/or olaparib on the level of DSBs in BRCA-wild type PEO4 cells. FIG. 21C depicts representative effects of DB4 and/or olaparib on the level of PD-L1 protein in BRCA-wild type PEO4 cells.

FIG. 22 depicts representative effects of DB4 and/or olaparib on PD-L1-positive populations of BRCA-mutated PEO1 and BRCA-wild type PEO4 cells. DB4 and/or olaparib increased the PD-L1-positive population of BRCA-wild type PEO4 cells in the presence of IFNγ.

FIG. 23 depicts representative effects of DB4 or triapine on peripheral blood mononuclear cells (PBMCs) containing T cells that were co-cultured with BRCA-mutated PEO1 and BRCA-wild type PEO4 cells and activated by CD3-CD28 beads. PEO1 cells suppressed CD8-positive activated T cells. DB4 or triapine-treated PEO4 cells also suppressed T cell activation (arrows).

FIG. 24 depicts representative effects of DB4 or triapine on peripheral blood mononuclear cells (PBMCs) containing T cells that were co-cultured with BRCA-mutated PEO1 and BRCA-wild type PEO4 cells and activated by CD3-CD28 beads. PEO1 cells increased PD1 and LAG3-positive exhausted T cells. DB4 or triapine-treated PEO4 cells also increased PD1 and LAG3-positive exhausted T cells (arrows).

FIG. 25 depicts representative effects of anti-PD-L1 antibody, DB4, and olaparib on the survivals of humanized NCG mice bearing PEO4ip xenografts. The anti-PD-L1 antibody enhanced the efficacy of DB4 and olaparib to suppress the progression of BRCA-wild type PEO4ip xenografts and prolong the survival time of NCG mice.

### DETAILED DESCRIPTION

The present invention is based, in part, on the unexpected discovery of compounds represented by Formulae (I) to (V) that inhibit ribonucleotide reductase (RNR), poly ADP-ribose polymerase (PARP), homologous recombination (HR) repair, or any combination thereof. Thus, in one aspect, the present invention relates, in part, to compounds represented by Formulae (I) to (V). In one aspect, the present invention relates to compositions comprising at least one compound represented by Formulae (I) to (V). In some aspects, the present invention also relates, in part, to a method of reducing or inhibiting RNR, PARP, HR repair, or any combination thereof using said compounds. In one aspect, the present invention relates, in part, to a method of inducing at least one double strand break (DBS) using said compounds. In some aspects, the present invention relates, in part, to a method of reducing or inhibiting HR repair, cyclin A2, Rad51 foci, or any combination thereof using said compounds. In one aspect, the present invention provides methods of increasing the level or activity of programmed death-ligand 1 (PD-L1) or at least one tumor antigen (e.g., tumor neoantigen) using said compounds. In another aspect, the present invention provides to methods of treating or preventing cancer (e.g., ovarian cancer, BRCA-wild type cancer, etc.) using said compounds.

### Definitions

Unless defined otherwise, all technical and scientific terms used herein have the same meaning as commonly understood by one of ordinary skill in the art to which this invention belongs. The skilled artisan will understand that any methods and materials similar or equivalent to those described herein can be used in the practice or testing of the present invention.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, for example numerical values and/or ranges, such as an amount, a temporal duration, and the like, is meant to encompass variations of ±20%, ±10%, ±5%, ±1%, or ±0.1% from the specified value, as such variations are appropriate to perform the disclosed methods. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein.

The term “compound,” as used herein, unless otherwise indicated, refers to any specific chemical compound disclosed herein. In one embodiment, the term also refers to stereoisomers and/or optical isomers (including racemic mixtures) or enantiomerically enriched mixtures of disclosed compounds.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

As used herein, the term “prodrug” refers to an agent that is converted into the parent drug in vivo. For example, the term “prodrug” refers to a derivative of a known direct acting drug, which derivative has enhanced delivery characteristics and therapeutic value as compared to the drug, and is transformed into the active drug by an enzymatic or chemical process. In some embodiments, “prodrug” refers to an inactive or relatively less active form of an active agent that becomes active by undergoing a chemical conversion through one or more metabolic processes. In one embodiment, upon in vivo administration, a prodrug is chemically converted to the biologically, pharmaceutically, or therapeutically active form of the compound. In another embodiment, a prodrug is enzymatically metabolized by one or more steps or processes to the biologically, pharmaceutically, or therapeutically active form of the compound. For example, the present compounds can be administered to a subject as a prodrug that includes an initiator bound to an active agent, and, by virtue of being degraded by a metabolic process, the active agent is released in its active form.

The term “tautomers” are constitutional isomers of organic compounds that readily interconvert by a chemical process (tautomerization).

The term “isomers” or “stereoisomers” refers to compounds, which have identical chemical constitution, but differ with regard to the arrangement of the atoms or groups in space.

As used herein, the term “alkyl,” or “alkyl group” by itself or as part of another substituent means, unless otherwise stated, a straight or branched chain hydrocarbon having from 1 to 24 carbon atoms. In some embodiments, the alkyl is a C₁-C₂₄ alkyl, a C₁-C₁₂ alkyl, a C₁-C₁₀ alkyl, a C₁-C₈ alkyl, a C₁-C₆ alkyl, a C₁-C₄ alkyl, or a C₁-C₃ alkyl. For example, an alkyl comprising up to 20 carbon atoms is a C₁-C₂₀ alkyl, an alkyl comprising up to 12 carbon atoms is a C₁-C₁₂ alkyl, an alkyl comprising up to 10 carbon atoms is a C₁-C₁₀ alkyl and an alkyl comprising up to 5 carbon atoms is a C₁-C₈ alkyl. A C₁-C₈ alkyl includes C₅ alkyls, C₄ alkyls, C₃ alkyls, C₂ alkyls and C₁alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₂₀ alkyl includes all the foregoing moieties, but also includes C₁, C₁₂, C₁₃, C₁₄, C₁₅, C₁₆, C₁₇, C₁₈, C₁₉, and C₂₀ alkyls. Non-limiting examples include methyl, ethyl, n-propyl, isopropyl, n-butyl, isobutyl, sec-butyl, tert-butyl, n-pentyl, neopentyl, n-hexyl, n-heptyl, n-octyl, n-nonyl, n-decyl, n-undecyl, n-dodecyl, and cyclopropylmethyl. Unless stated otherwise specifically in the specification, an alkyl group can be optionally substituted.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkylene” by itself or as part of another molecule means a divalent radical derived from an alkane, as exemplified by (—CH₂—)ₙ. In some embodiments, the alkylene is a C₁-C₂₄ alkylene, a C₂-C₁₂ alkylene, a C₁-C₁₀ alkylene, a C₁-C₈ alkylene, a C₁-C₆ alkylene, a C₁-C₄ alkylene, or a C₁-C₃ alkylene. Non-limiting examples of C₁-C₂₀ alkylene include methylene, ethylene, propylene, n-butylene, ethenylene, propenylene, n-butenylene, propynylene, n-butynylene, and the like. The points of attachment of the alkylene chain to the rest of the molecule can be through one carbon or any two carbons within the chain. Unless stated otherwise specifically in the specification, an alkylene chain can be optionally substituted. The term “alkylene,” unless otherwise noted, is also meant to include those groups described below as “heteroalkylene.”

“Alkenyl” or “alkenyl group” refers to a straight or branched hydrocarbon chain having from 2 to 24 carbon atoms, and having one or more carbon-carbon double bonds. Each alkenyl group is attached to the rest of the molecule by a single bond. Alkenyl group comprising any number of carbon atoms from 2 to 24 are included. In some embodiments, the alkenyl is a C₂-C₂₄ alkenyl, a C₂-C₁₂ alkenyl, a C₂-C₁₀ alkenyl, a C₂-C₈ alkenyl, a C₂-C₆ alkenyl, a C₂-C₄ alkenyl, or a C₂-C₃ alkenyl. An alkenyl group comprising up to 20 carbon atoms is a C₂-C₂₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₂₄ alkenyl includes all the foregoing moieties, but also includes C₁, C₁₂, C₁₃, C₁₄, C₁₅, C₁₆, C₁₇, C₁₈, C₁₉, and C₂₄ alkenyls. Non-limiting examples of C₂-C₂₄ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkynyl” or “alkynyl group” refers to a straight or branched hydrocarbon chain having from 2 to 24 carbon atoms, and having one or more carbon-carbon triple bonds. Each alkynyl group is attached to the rest of the molecule by a single bond. In some embodiments, the alkynyl is a C₂-C₂₄ alkynyl, a C₂-C₁₂ alkynyl, a C₂-C₁₀ alkynyl, a C₂-C₈ alkynyl, a C₂-C₆ alkynyl, a C₂-C₄ alkynyl, or a C₂-C₃ alkynyl. Alkynyl group comprising any number of carbon atoms from 2 to 24 are included. An alkynyl group comprising up to 24 carbon atoms is a C₂-C₂₄ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₁, C₈, C₉ and C₁₀ alkynyls. Similarly, a C₂-C₂₄ alkynyl includes all the foregoing moieties, but also includes C₁, C₁₂, C₁₃, C₁₄, C₁₅, C₁₆, C₁₇, C₁₈, C₁₉, and C₂₄ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

As used herein, the terms “alkoxy,” “alkylamino” and “alkylthio” are used in their conventional sense, and refer to alkyl groups linked to molecules via an oxygen atom, an amino group, a sulfur atom, respectively.

As used herein, the term “alkoxy” employed alone or in combination with other terms means, unless otherwise stated, refers to a group of the formula —ORₐ where Rₐ is an alkyl, alkenyl or alknyl group having from 1 to 24 carbon atoms, as defined above, connected to the rest of the molecule via an oxygen atom, such as, for example, methoxy, ethoxy, 1-propoxy, 2-propoxy (isopropoxy) and the higher homologs and isomers. Unless stated otherwise specifically in the specification, an alkoxy group can be optionally substituted.

As used herein, the term “halo” or “halogen” alone or as part of another substituent means, unless otherwise stated, a fluorine, chlorine, bromine, or iodine atom. As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

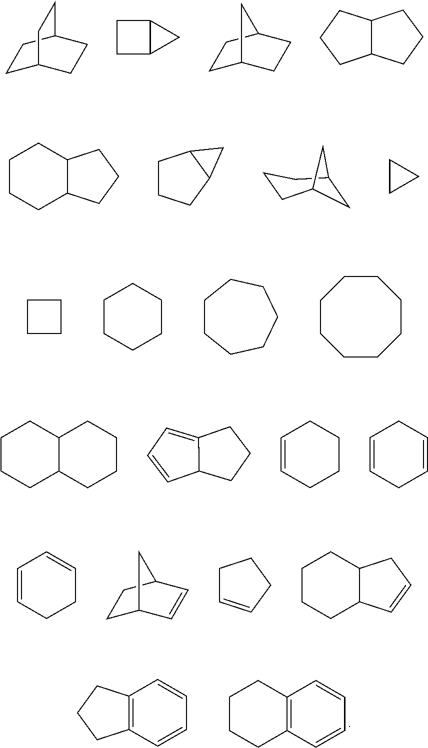

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

“Cycloalkenyl” refers to a stable non aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyls include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—Rₐ where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

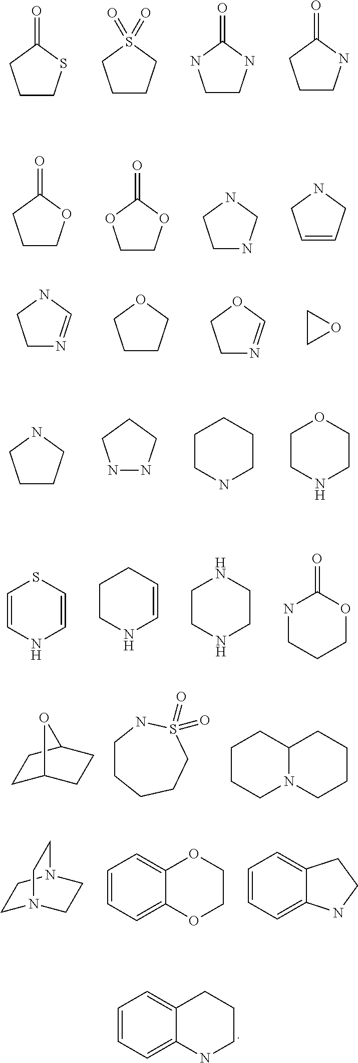

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “aryl-(C₁-C₄)alkyl” means a functional group wherein a one to three carbon alkylene chain is attached to an aryl group, e.g., —CH₂CH₂-phenyl. Preferred is aryl-CH₂— and aryl-CH(CH₃)—. The term “substituted aryl-(C₁-C₄)alkyl” means an aryl-(C₁-C₄)alkyl functional group in which the aryl group is substituted. Preferred is substituted aryl(CH₂)—. Similarly, the term “heteroaryl-(C₁-C₄)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₄)alkyl” means a heteroaryl-(C₁-C₄)alkyl functional group in which the heteroaryl group is substituted. Preferred is substituted heteroaryl-(CH₂)—.

As used herein, the term “heteroaryl” or “heteroaromatic” refers to a 5 to 24 membered ring system comprising hydrogen atoms, one to fourteen carbon atoms, one to six heteroatoms selected from the group consisting of nitrogen, oxygen and sulfur, and at least one aromatic ring. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. A polycyclic heteroaryl may include one or more rings that are partially saturated. Examples include the following moieties:

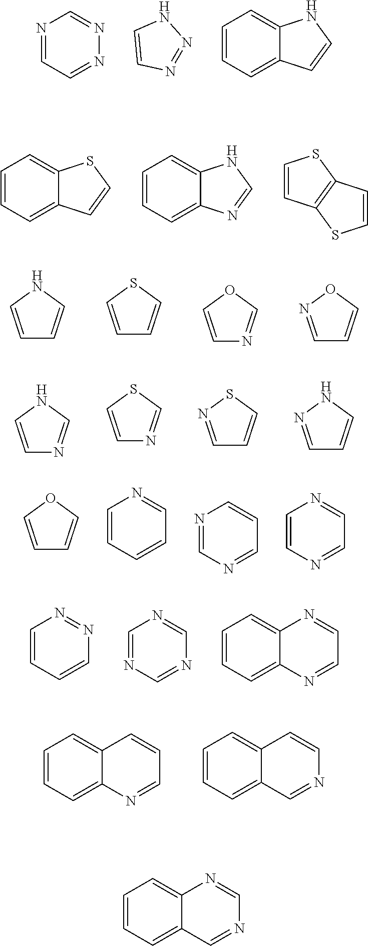

wherein any hydrogen atom in the above groups may be replaced by a bond to the molecule.

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl. Unless stated otherwise specifically in the specification, a heteroaryl group can be optionally substituted.

The aforementioned listing of heterocyclyl and heteroaryl moieties is intended to be representative and not limiting.

The term “amino” refers to a group of the formula —NRₐRₐ, —NHRₐ, or —NH₂, where each Rₐ is, independently, an alkyl, alkenyl or alkynyl group as defined above containing 1 to 24 carbon atoms. Unless stated otherwise specifically in the specification, an alkylamino group can be optionally substituted.

“Alkylamino” refers to a group of the formula —NHRₐ or —NRₐRₐ where each Rₐ is, independently, an alkyl, alkenyl or alkynyl group as defined above containing 1 to 24 carbon atoms. Unless stated otherwise specifically in the specification, an alkylamino group can be optionally substituted.

The term “cyano” refers to a group of the formula —CN group.

The term “imino” refers to a group of the formula ═NH group.

The term “nitro” refers to a group of the formula —NO₂ group.

The term “oxo” refers to a group of the formula the ═O group.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means any of the above groups (i.e., alkyl, alkylene, alkenyl, alkynyl, alkoxy, aryl, carbocyclyl, cycloalkyl, cycloalkenyl, cycloalkynyl, heterocyclyl, and/or heteroaryl) wherein at least hydrogen atom is replaced by a bond to a non-hydrogen atom or group of atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. “Substituted” also means any of the above groups in which one or more hydrogen atoms are replaced by a higher-order bond (e.g., a double- or triple-bond) to a heteroatom such as oxygen in oxo, carbonyl, carboxyl, carboxylic acid and ester groups; and nitrogen in groups such as imines, oximes, hydrazones, and nitriles. For example, “substituted” includes any of the above groups in which one or more hydrogen atoms are replaced with, for example, —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with, for

example, —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently selected from any of the above groups, including but not limited to: hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to any of the above groups, including but not limited to amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group.

In addition, each of the foregoing substituents can also be optionally substituted with one or more of the above substituents.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]2, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration. The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration.

Non-limiting examples of agents suitable for formulation with the, e.g., compounds provided by the instant invention include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

A “therapeutic” treatment is a treatment administered to a subject who exhibits signs or symptoms of pathology disease or disorder, for the purpose of diminishing or eliminating those signs or symptoms.

As used herein, the terms “effective amount,” “pharmaceutically effective amount” and “therapeutically effective amount” refer to a sufficient amount of an agent to provide the desired biological or physiologic result. That result may be reduction and/or alleviation of a sign, a symptom, or a cause of a disease or disorder, or any other desired alteration of a biological system. An appropriate effective amount in any individual case may be determined by one of ordinary skill in the art using routine experimentation.

As used herein, the term “potency” refers to the dose needed to produce half the maximal response (ED₅₀).

As used herein, the term “efficacy” refers to the maximal effect (Eₘₐₓ) achieved within an assay.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “cancer” refers to any of various types of malignant neoplasms, most of which invade surrounding tissues, may metastasize to several sites and are likely to recur after attempted removal and to cause death of the patient unless adequately treated. As used herein, neoplasia comprises cancer. Representative cancers include, for example, BRCA-wild type cancer, squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias, including non-acute and acute leukemias, such as acute myelogenous leukemia, acute lymphocytic leukemia, acute promyelocytic leukemia (APL), acute T-cell lymphoblastic leukemia, T-lineage acute lymphoblastic leukemia (T-ALL), adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas, among others, which may be treated by one or more compounds of the present invention.

A disease or disorder is “alleviated” if the severity of a sign or symptom of the disease or disorder, the frequency with which such a sign or symptom is experienced by a patient, or both, is reduced.

The term “inhibit,” as used herein, means to suppress or block an activity or function by at least about ten percent relative to a control value. Preferably, the activity is suppressed or blocked by 50% compared to a control value, more preferably by 75%, and even more preferably by 95% or more.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell from a patient (e.g., for diagnosis or ex vivo applications), who has a disease or disorder contemplated herein, a sign or symptom of a disease or disorder contemplated herein or the potential to develop a disease or disorder contemplated herein, with the purpose to cure, heal, alleviate, relieve, alter, remedy, ameliorate, improve or affect a disease or disorder contemplated herein, the signs or symptoms of a disease or disorder contemplated herein or the potential to develop a disease or disorder contemplated herein. Such treatments may be specifically tailored or modified, based on knowledge obtained from the field of pharmacogenomics. To “treat” a disease as the term is used herein, means to reduce the frequency or severity of at least one sign or symptom of a disease or disorder experienced by a subject.

“Parenteral” administration of a composition includes, e.g., subcutaneous (s.c.), intravenous (i.v.), intramuscular (i.m.), or intrasternal injection, or infusion techniques.

In one aspect, the terms “co-administered” and “co-administration” as relating to a subject refer to administering to the subject a compound useful within the invention, or salt thereof, along with a compound that may also treat any of the diseases contemplated within the invention. In one embodiment, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the unexpected discovery of compounds represented by Formulae (I) to (V) that inhibit ribonucleotide reductase (RNR), poly ADP-ribose polymerase (PARP), homologous recombination (HR) repair, or any combination thereof. Thus, in one aspect, the present invention relates, in part, to compounds represented by Formulae (I) to (V). In one aspect, the present invention relates to compositions comprising at least one compound represented by Formulae (I) to (V). In some aspects, the present invention also relates, in part, to a method of reducing or inhibiting RNR, PARP, HR repair, or any combination thereof using said compounds. In one aspect, the present invention relates, in part, to a method of inducing at least one double strand break (DBS) using said compounds. In some aspects, the present invention relates, in part, to a method of reducing or inhibiting HR repair, cyclin A2, Rad51 foci, or any combination thereof using said compounds. In one aspect, the present invention provides methods of increasing the level or activity of programmed death-ligand 1 (PD-L1) or at least one tumor antigen (e.g., tumor neoantigen) using said compounds. In another aspect, the present invention provides to methods of treating or preventing cancer (e.g., ovarian cancer, BRCA-wild type cancer, etc.) using said compounds.

### Compounds

In one aspect, the compound of the present invention is a compound represented by one of Formula (I) or a racemate, enantiomer, diastereomer, pharmaceutically acceptable salt, solvate, hydrate, or derivative thereof:

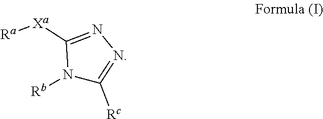

In some embodiments, X^{a} is O, S, —SO₂, —N(R^{x}), —C(R^{x})(R^{y}), or —C═R^{z}. For example, in one embodiment, X^{a} is S.

In some embodiments, each occurrence of R^{a}, R^{b}, R^{c}, R^{x}, and R^{y} is independently hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, or any combination thereof. In some embodiments, R^{z} is O, S, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, or any combination thereof.

In some embodiments, each occurrence of R^{a}, R^{b}, R^{c}, R^{x}, R^{y}, and R^{z} is independently optionally substituted. In one embodiment, R^{a} is substituted. In one embodiment, R^{b} is substituted. In one embodiment, R^{c} is substituted. In one embodiment, R^{x} is substituted. In one embodiment, R^{y} is substituted. In one embodiment, R^{z} is substituted.

In some embodiments, n is an integer represented by 0 to 5. For example, in one embodiment, n is 1. In one embodiment, n is 2. In one embodiment, n is 3. In one embodiment, n is 4. In one embodiment, n is 5. In one embodiment, n is 0.

For example, in some embodiments, R^{a}—X^{a} is

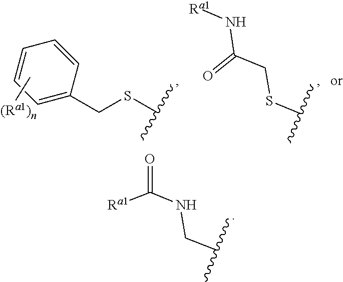

In some embodiments, R^{a} is aryl alkyl or substituted aryl alkyl. In some embodiments, R^{a} is chlorobenzyl.

In one embodiment, R^{b} is alkenyl. For example, in one embodiment, the alkenyl is allyl. Thus, in one embodiment, R^{b} is allyl. In some embodiments, R^{b} is

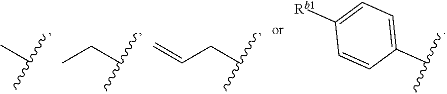

In some embodiments, R^{c} is alkyl, alkyl aryl, heteroalkyl, or heterocycloalkyl. In one embodiment, R^{c} is substituted heterocycloalkyl. In some embodiments, the heterocycloalkyl is substituted with alkyl, alkyl aryl, alkyl heteroaryl, or alkyl bicyclic heteroaryl. In one embodiment, the heterocycloalkyl is piperidine. In one embodiment, the alkyl is ethyl. In some embodiments, the heteroaryl is pyridinyl, benzothiazolyl, and thiophenyl. In some embodiments, R^{c} is

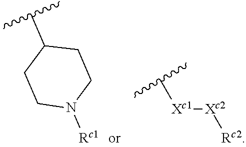

Thus, in some embodiments, R^{c} is piperidine substituted with ethyl,

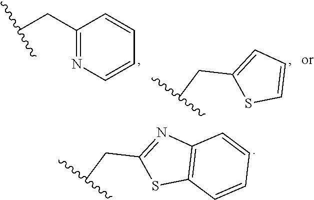

Thus, in one embodiment, the compound represented by Formula (I) is a compound represented by Formula (Ia)

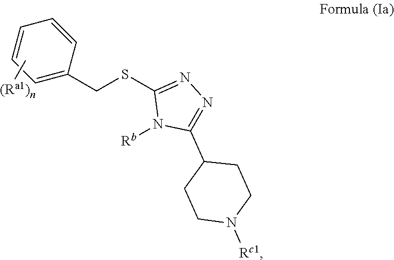

or a racemate, enantiomer, diastereomer, pharmaceutically acceptable salt, solvate, hydrate, or derivative thereof.

In one embodiment, the compound represented by Formula (I) is a compound represented by Formula (Ib)

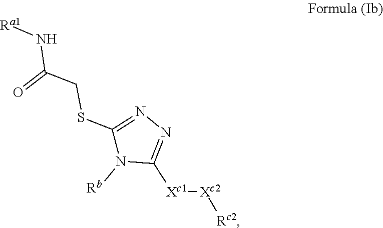

or a racemate, enantiomer, diastereomer, pharmaceutically acceptable salt, solvate, hydrate, or derivative thereof.

In one embodiment, the compound represented by Formula (I) is a compound represented by Formula (Ic)

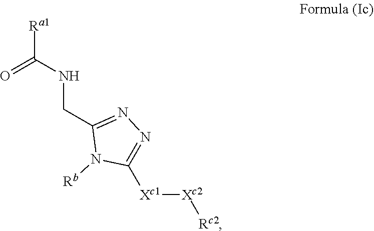

or a racemate, enantiomer, diastereomer, pharmaceutically acceptable salt, solvate, hydrate, or derivative thereof.

In some embodiments, each occurrence of R^{a1} is independently hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, —NO₂, —CN, thiol, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, or sulfoxyalkyl-heteroaryl. For example, in some embodiments, each occurrence of R^{a1} is independently selected from hydrogen, deuterium, fluoro, chloro, bromo, iodo, alkyl, aryl, aryl alkyl, heteroaryl, heteroaryl alkyl, aminoaryl, aminoaryl alkyl, aminoheteroaryl, aminoheteroaryl alkyl, thioaryl, thioaryl alkyl, thioheteroaryl, thioheteroaryl alkyl, or any combination thereof. For example, in one embodiment, R^{a1} is hydrogen or halogen. In some embodiments, halogen is Cl, F, I, or Br.

In some embodiments, R^{b1}, R^{c1}, and R^{c2} are each independently hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, —NO₂, —CN, thiol, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, or sulfoxyalkyl-heteroaryl. For example, in some embodiments, R^{b1} is selected from hydrogen, deuterium, fluoro, chloro, bromo, iodo, alkyl, or any combination thereof. In some embodiments, R^{c1} and R^{c2} are independently selected from hydrogen, deuterium, fluoro, chloro, bromo, iodo, alkyl, aryl, aryl alkyl, heteroaryl, heteroaryl alkyl, alkoxycarbonyl, acyl, hydroxyl, hydroxyalkyl, hydroxyaryl, hydroxyalkyl-aryl, hydroxyheteroaryl, hydroxyalkyl-heteroaryl, alkoxy, carboxyl, carboxylate, ester, aminoaryl, aminoaryl alkyl, aminoheteroaryl, aminoheteroaryl alkyl, thioaryl, thioaryl alkyl, thioheteroaryl, thioheteroaryl alkyl, or any combination thereof. In some embodiments, R^{c1} is hydrogen, methyl, ethyl,

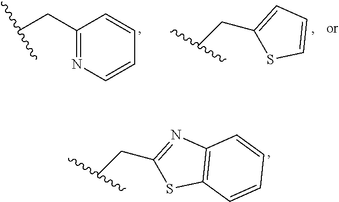 [SMILES: *Cc1ccccn1 ; *Cc1cccs1 ; *Cc1nc2ccccc2s1]

In some embodiments, X^{c1} and X^{c2} are independently selected from O, S, —SO₂, —N(R^{x}), or —C(R^{x})(R^{y}).

In some embodiments, R^{x} and R^{y} are independently selected from hydrogen or alkyl.

In some embodiments, R^{a1}, R^{b1}, R^{c1}, R^{c2}, R^{x}, and R^{y} are each independently optionally substituted. In one embodiment, R^{a1} is substituted. In one embodiment, R^{b1} is substituted. In one embodiment, R^{c1} is substituted. In one embodiment, R^{c2} is substituted. In one embodiment, R^{x} is substituted. In one embodiment, R^{y} is substituted.

For example, in some embodiments, the compound represented by Formula (Ia) is

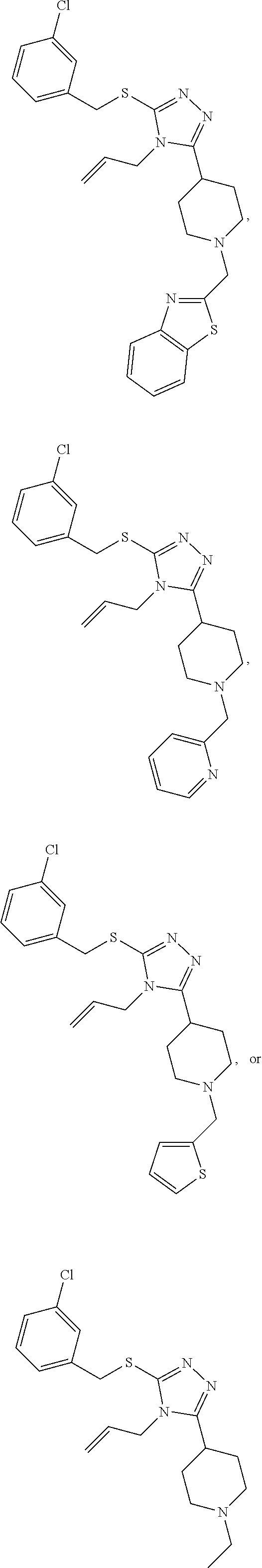 [SMILES: C=CCn1c(SCc2cccc(Cl)c2)nnc1C1CCN(Cc2nc3ccccc3s2)CC1 ; C=CCn1c(SCc2cccc(Cl)c2)nnc1C1CCN(Cc2ccccn2)CC1 ; C=CCn1c(SCc2cccc(Cl)c2)nnc1C1CCN(Cc2cccs2)CC1 ; C=CCn1c(SCc2cccc(Cl)c2)nnc1C1CCN(CC)CC1]

In some embodiments, the compound represented by Formula (Ib) is

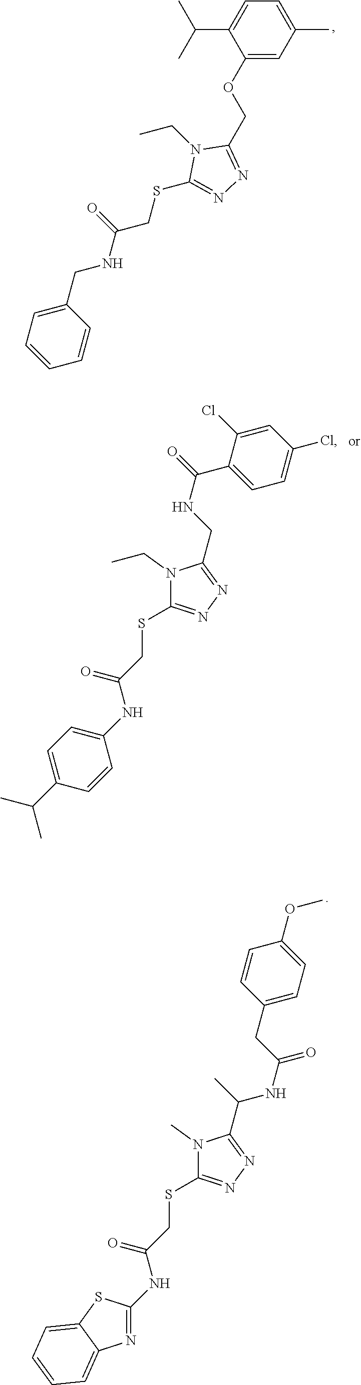

In one embodiment, the compound represented by Formula (Ic) is

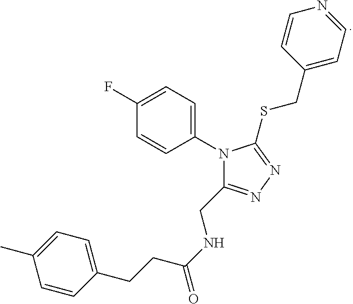 [SMILES: Cc1ccc(CCC(=O)NCc2nnc(SCc3ccncc3)n2-c2ccc(F)cc2)cc1]

In one aspect, the compound of the present invention is a compound represented by one of Formula (II) or a racemate, enantiomer, diastereomer, pharmaceutically acceptable salt, solvate, hydrate, or derivative thereof:

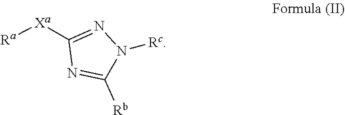

In some embodiments, X^{a} is O, S, —SO₂, —N(R^{x}), —C(R^{x})(R^{y}), or —C═R^{z}. For example, in one embodiment, X^{a} is S.

In some embodiments, each occurrence of R^{a}, R^{b}, R^{c}, R^{x}, and R^{y} is independently hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, or any combination thereof.

In some embodiments, R^{z} is O, S, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, or any combination thereof.

In some embodiments, each occurrence of R^{a}, R^{b}, R^{c}, R^{x}, R^{y}, and R^{z} is independently optionally substituted. In one embodiment, R^{a} is substituted. In one embodiment, R^{b} is substituted. In one embodiment, R^{c} is substituted. In one embodiment, R^{x} is substituted. In one embodiment, R^{y} is substituted. In one embodiment, R^{z} is substituted.

In some embodiments, n is an integer represented by 0 to 5. For example, in one embodiment, n is 1. In one embodiment, n is 2. In one embodiment, n is 3. In one embodiment, n is 4. In one embodiment, n is 5. In one embodiment, n is 0.

For example, in some embodiments, R^{a}—X^{a} is

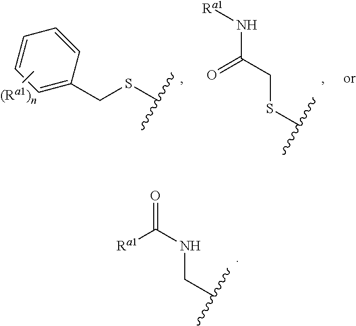

In some embodiments, R^{a} is aryl alkyl or substituted aryl alkyl. In some embodiments, R^{a} is chlorobenzyl.

In one embodiment, R^{b} is alkenyl. For example, in one embodiment, the alkenyl is allyl. Thus, in one embodiment, R^{b} is allyl. In some embodiments, R^{b} is

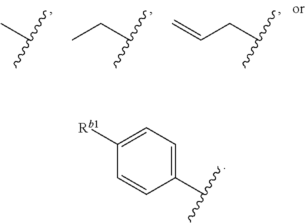

In some embodiments, R^{c} is alkyl, alkyl aryl, heteroalkyl, or heterocycloalkyl. In one embodiment, R^{c} is substituted heterocycloalkyl. In some embodiments, the heterocycloalkyl is substituted with alkyl, alkyl aryl, alkyl heteroaryl, or alkyl bicyclic heteroaryl. In one embodiment, the heterocycloalkyl is piperidine. In one embodiment, the alkyl is ethyl. In some embodiments, the heteroaryl is pyridinyl, benzothiazolyl, and thiophenyl. In some embodiments, R^{c} is

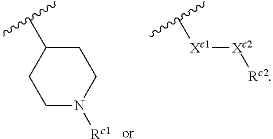

Thus, in some embodiments, R^{c} is piperidine substituted with ethyl,

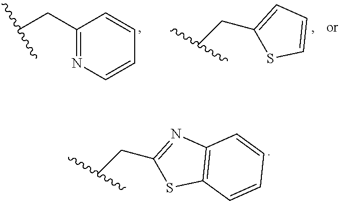 [SMILES: *Cc1ccccn1 ; *Cc1cccs1 ; *Cc1nc2ccccc2s1]

In one aspect, the compound of the present invention is a compound represented by one of Formula (III) or a racemate, enantiomer, diastereomer, pharmaceutically acceptable salt, solvate, hydrate, or derivative thereof

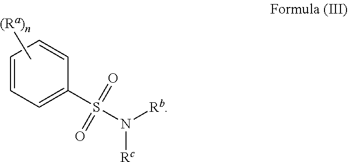

In some embodiments, each occurrence of R^{a} is independently hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, —NO₂, —CN, thiol, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, or any combination thereof. For example, in some embodiments, R^{a} is hydrogen, halogen, alkyl, aryl, or any combination thereof.

In some embodiments, R^{b} and R^{c} are each independently hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, —NO₂, —CN, thiol, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, or any combination thereof. For example, in some embodiments, R^{b} is hydrogen, alkyl, or aryl. In some embodiments, R^{c} is hydrogen, alkyl, or aryl.

In some embodiments, R^{b} and R^{c} are optionally joint to form a cycle. In one embodiment, R^{b} and R^{c} are joint to form a cycle.

In some embodiments, R^{a}, R^{b}, and R^{c} are each independently optionally substituted. In one embodiment, R^{a} is substituted. In one embodiment, R^{b} is substituted. In one embodiment, R^{c} is substituted.

In some embodiments, n is an integer represented by 0 to 5. For example, in one embodiment, n is 1. In one embodiment, n is 2. In one embodiment, n is 3. In one embodiment, n is 4. In one embodiment, n is 5. In one embodiment, n is 0.

For example, in some embodiments, the compound represented by Formula (III) is

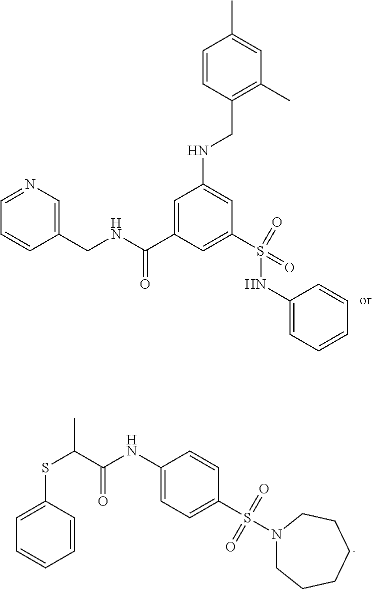 [SMILES: Cc1ccc(CNc2cc(C(=O)NCc3cccnc3)cc(S(=O)(=O)Nc3ccccc3)c2)c(C)c1 ; CC(Sc1ccccc1)C(=O)Nc1ccc(S(=O)(=O)N2CCCCCC2)cc1]

In another aspect, the compound of the present invention is a compound represented by one of Formula (IV) or a racemate, enantiomer, diastereomer, pharmaceutically acceptable salt, solvate, hydrate, or derivative thereof

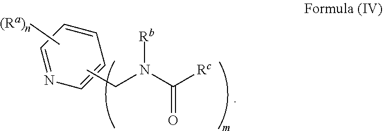

In some embodiments, each occurrence of R^{a} is independently hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, —NO₂, —CN, thiol, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, or any combination thereof.

In some embodiments, R^{b} and R^{c} are each independently hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, —NO₂, —CN, thiol, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, or any combination thereof.

In some embodiments, R^{a}, R^{b}, and R^{c} are each independently optionally substituted. In one embodiment, R^{a} is substituted. In one embodiment, R^{b} is substituted. In one embodiment, R^{c} is substituted.

In some embodiments, n is an integer represented by 0 to 5. For example, in one embodiment, n is 1. In one embodiment, n is 2. In one embodiment, n is 3. In one embodiment, n is 4. In one embodiment, n is 5. In one embodiment, n is 0.

In some embodiments, m is an integer represented by 0 to 5. For example, in one embodiment, m is 1. In one embodiment, m is 2. In one embodiment, m is 3. In one embodiment, m is 4. In one embodiment, m is 5. In one embodiment, m is 0.

Thus, in one embodiment, the compound represented by Formula (IV) is a compound represented by Formula (IVa)

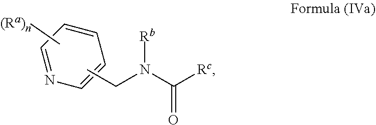

or a racemate, enantiomer, diastereomer, pharmaceutically acceptable salt, solvate, hydrate, or derivative thereof.

In yet another aspect, the compound of the present invention is a compound represented by one of Formula (V) or a racemate, enantiomer, diastereomer, pharmaceutically acceptable salt, solvate, hydrate, or derivative thereof.

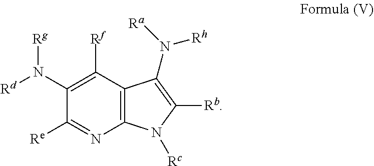

In some embodiments, R^{a}, R^{b}, R^{c}, R^{d}, R^{e}, R^{f}, R^{g}, and R^{h} are each independently hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, or any combination thereof.

In some embodiments, R^{a}, R^{b}, R^{c}, R^{d}, R^{e}, R^{f}, R^{g}, and R^{h} are each independently optionally substituted. In one embodiment, R^{a} is substituted. In one embodiment, R^{b} is substituted. In one embodiment, R^{c} is substituted. In one embodiment, R^{d} is substituted. In one embodiment, R^{e} is substituted. In one embodiment, R^{f} is substituted. In one embodiment, R& is substituted. In one embodiment, R^{h} is substituted.

For example, in some embodiments, R^{g} and R^{h} are hydrogen.

In various aspects, the compound of the present invention is a ribonucleotide reductase (RNR) inhibitor. In one embodiment, the compound represented by Formula (I) is a RNR inhibitor. In one embodiment, the compound represented by Formula (II) is a RNR inhibitor. In one embodiment, the compound represented by Formula (III) is a RNR inhibitor. In one embodiment, the compound represented by Formula (IV) is a RNR inhibitor. In one embodiment, the compound represented by Formula (V) is a RNR inhibitor.

The compounds of the present invention may be synthesized using techniques well-known in the art of organic synthesis. The starting materials and intermediates required for the synthesis may be obtained from commercial sources or synthesized according to methods known to those skilled in the art.

For example, compounds of the invention may be prepared by the general schemes described herein, using the synthetic method known by those skilled in the art.

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

In one embodiment, the compounds of the invention may exist as tautomers. All tautomers are included within the scope of the compounds presented herein.

In one embodiment, compounds described herein are prepared as prodrugs. A “prodrug” refers to an agent that is converted into the parent drug in vivo. In one embodiment, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

In one embodiment, sites on, for example, the aromatic ring portion of compounds of the invention are susceptible to various metabolic reactions. Incorporation of appropriate substituents on the aromatic ring structures may reduce, minimize or eliminate this metabolic pathway. In one embodiment, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference in their entirety). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In one embodiment, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In one embodiment, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In one embodiment, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

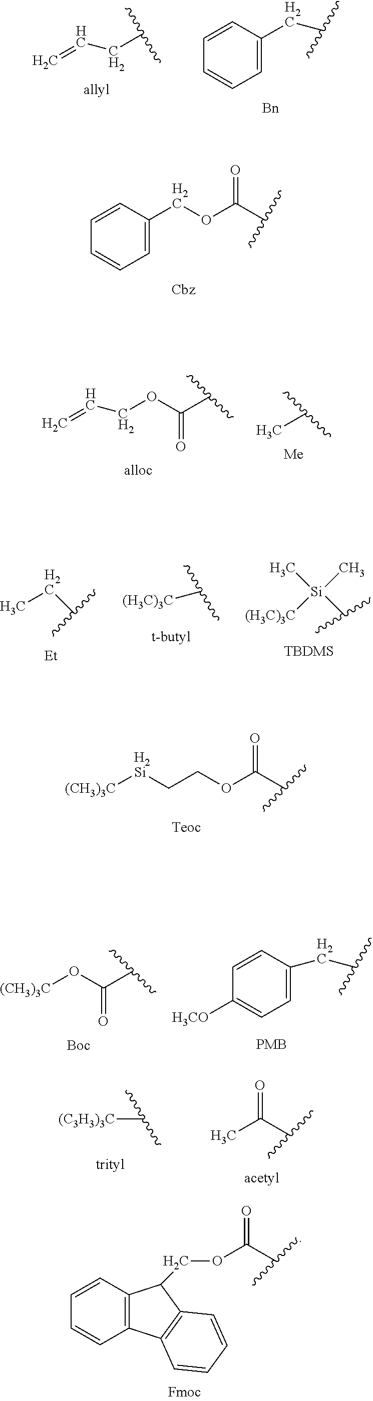

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Compositions

As described elsewhere herein, the invention is based, in part, on the discovery that the compounds of the present invention inhibit RNR, PARP, cyclin A2, Rad51 foci, and/or HR repair, induce at least one DSB, regulate the level or activity of checkpoint kinase 1 (Chk1), ataxia telangiectasia and Rad3-related protein (ATR), replication protein A32 (RPA32), histone H2A, such as H2AX, histone H1, cyclin-dependent kinase 1 (CDK1), and/or cyclin-dependent kinase 2 (CDK2), by regulating the phosphorylation of Chk1, ATR, RPA32, histone H2A, such as H2AX, histone H1, CDK1, and/or CDK2, or any combination thereof. Therefore, compounds represented by Formulae (I) to (V), analogs of compounds represented by Formulae (I) to (V), or prodrugs of compounds represented by Formulae (I) to (V) are useful therapeutics for treating cancer (e.g., ovarian cancer, BRCA-wild type cancer, etc.).

In one embodiment, the invention provides an inhibitor of RNR, PARP, cyclin A2, Rad51 foci, HR repair, CDK1, CDK2, or any combination thereof. In various embodiments, the present invention includes compositions for inhibiting the RNR, PARP, cyclin A2, Rad51 foci, HR repair, CDK1, CDK2, or any combination thereof in a subject, a tissue, or an organ in need thereof. In one embodiment, the composition comprises at least one compound represented by one of Formulae (I) to (V).

In some embodiments, the composition optionally further comprises a PARP inhibitor. Examples of such PARP inhibitors include, but are not limited to, olaparib, rucaparib, niraparib, talazoparib, veliparib, pamiparib, CEP 9722, E7016, iniparib, 3-aminobenzamide, or any combination thereof. In one embodiment, the PARP inhibitor is olaparib.

### Methods

In one aspect, the present invention provides methods of reducing or inhibiting the level or activity of PARP in a subject in need thereof. In one embodiment, the method comprises administering to the subject an effective amount of at least one compound of the present invention or a composition thereof.

In one aspect, the present invention provides methods of reducing or inhibiting the level or activity of RNR in a subject in need thereof. In one embodiment, the method comprises administering to the subject an effective amount of at least one compound of the present invention or a composition thereof.

In one aspect, the present invention provides methods of reducing or inhibiting HR repair in a subject in need thereof. In one embodiment, the method comprises administering to the subject an effective amount of at least one compound of the present invention or a composition thereof.

In one aspect, the present invention provides methods of reducing or inhibiting the level or activity of cyclin A2 in a subject in need thereof. In one embodiment, the method comprises administering to the subject an effective amount of at least one compound of the present invention or a composition thereof.

In one aspect, the present invention provides methods of reducing or inhibiting the level or activity of Rad51 foci in a subject in need thereof. In one embodiment, the method comprises administering to the subject an effective amount of at least one compound of the present invention or a composition thereof.

In one aspect, the present invention provides methods of reducing or inhibiting RNR, PARP, cyclin A2, Rad51 foci, HR repair, or any combination thereof. In one embodiment, the method comprises administering to the subject an effective amount of at least one compound of the present invention or a composition thereof.

In one aspect, the present invention provides methods of inducing at least one DSB in a subject in need thereof. In one embodiment, the method comprises administering to the subject an effective amount of at least one compound of the present invention or a composition thereof.

In various aspects, the present invention provides methods of regulating the level or activity of Chk1, ATR, RPA32, histone H2A, such as H2AX, histone H1, CDK1, CDK2, or any combination thereof in a subject in need thereof. In some embodiments, the present invention provides methods of reducing the level or activity of Chk1, ATR, RPA32, histone H2A, such as H2AX, histone H1, CDK1, CDK2, or any combination thereof in a subject in need thereof. In some embodiments, the present invention provides methods of increasing the level or activity of Chk1, ATR, RPA32, histone H2A, such as H2AX, histone H1, CDK1, CDK2, or any combination thereof in a subject in need thereof. In one embodiment, the method comprises administering to the subject an effective amount of at least one compound of the present invention or a composition thereof.

In various aspects, the present invention provides methods of regulating phosphorylation of Chk1, ATR, RPA32, histone H2A, such as H2AX, histone H1, CDK1, CDK2, or any combination thereof in a subject in need thereof. In some embodiments, the present invention provides methods of decreasing phosphorylation of Chk1, ATR, RPA32, histone H2A, such as H2AX, histone H1, CDK1, CDK2, or any combination thereof in a subject in need thereof. In some embodiments, the present invention provides methods of increasing phosphorylation of Chk1, ATR, RPA32, histone H2A, such as H2AX, histone H1, CDK1, CDK2, or any combination thereof in a subject in need thereof. In one embodiment, the method comprises administering to the subject an effective amount of at least one compound of the present invention or a composition thereof.

In one aspect, the present invention provides methods of increasing the level or activity of programmed death-ligand 1 (PD-L1) in a subject in need thereof. In one embodiment, the method comprises administering to the subject an effective amount of at least one compound of the present invention or a composition thereof.

In one aspect, the present invention provides methods of increasing the level or activity of at least one tumor antigen. In one embodiment, the tumor antigen is a tumor neoantigen. In one embodiment, the method comprises administering to the subject an effective amount of at least one compound of the present invention or a composition thereof.

In one aspect, the present invention provides methods of inhibiting RNR, PARP, cyclin A2, Rad51 foci, HR repair, or any combination thereof, inducing at least one DSB, regulating the level or activity of Chk1, ATR, RPA32, histone H2A, H2AX, histone H1, CDK1, CDK2, or any combination thereof, regulating the phosphorylation of Chk1, ATR, RPA32, histone H2A, such as H2AX, histone H1, CDK1, and CDK2, or any combination thereof, and/or increasing the level or activity of PD-L1, at least one tumor antigen, such as neoantigen, or any combination thereof.

In one aspect, the present invention provides methods of administering at least one compound of the present invention or a composition thereof to a subject. In one embodiment, the subject has cancer. In one embodiment, cancer includes, but is not limited to, an ovarian cancer, BRCA-wild type cancer, or a combination thereof.

In one aspect, the present invention provides methods of treating cancer in a subject in need thereof. In one embodiment, the method comprises administering to the subject an effective amount of at least one compound of the present invention or a composition thereof. In one embodiment, cancer includes, but is not limited to, an ovarian cancer, BRCA-wild type cancer, or a combination thereof.

The disclosed compounds can be used to slow the rate of primary tumor growth. The disclosed compounds can also be used to prevent, abate, minimize, control, and/or lessen tumor metastasis in humans and animals. The disclosed compounds when administered to a subject in need of treatment can be used to stop the spread of cancer cells. As such, the compounds disclosed herein can be administered as part of a combination therapy with one or more drugs or other pharmaceutical agents. When used as part of the combination therapy, the decrease in metastasis and reduction in primary tumor growth afforded by the disclosed compounds allows for a more effective and efficient use of any pharmaceutical or drug therapy being used to treat the patient. In addition, control of metastasis by the disclosed compound affords the subject a greater ability to concentrate the disease in one location.

The following are non-limiting examples of cancers that can be treated by the disclosed methods and compositions: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer;

Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytotna/Malignant Glioma; Craniopharyngioma; Ependymoblastoma; Ependymoma; Medulloblastoma;

Medulloepithelioma; Pineal Parenchymal Tumors of intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors;

Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocvtoma of Bone and Osteosarcoma; Medulloblastoma; Melanoma; Melanoma, intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis; Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; BRCA-wild type cancer, Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Celt Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor.

The composition of the invention may be administered to a patient or subject in need in a wide variety of ways. Modes of administration include intraoperatively intravenous, intravascular, intramuscular, subcutaneous, intracerebral, intraperitoneal, soft tissue injection, surgical placement, arthroscopic placement, and percutaneous insertion, e.g., direct injection, cannulation or catheterization. Any administration may be a single application of a composition of invention or multiple applications. Administrations may be to single site or to more than one site in the individual to be treated. Multiple administrations may occur essentially at the same time or separated in time.

### Combination Therapies

The compounds of the present invention may be useful in combination with one or more additional compounds. In certain embodiments, these additional compounds may comprise compounds of the present invention or therapeutic agents. In certain embodiments, the anti-cancer agent may comprise compounds useful for treating cancer. Such compounds include, but are not limited to, compounds which are known to treat, prevent, or reduce the symptoms of cancer.

In one embodiment, the invention includes a method comprising administering a combination of inhibitor compounds described herein. In certain embodiments, the method has an additive effect, wherein the overall effect of administering the combination of inhibitor compounds is approximately equal to the sum of the effects of administering each individual inhibitor compound. In other embodiments, the method has a synergistic effect, wherein the overall effect of administering a combination of inhibitor compounds is greater than the sum of the effects of administering each individual inhibitor compound. A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

The method comprises administering a combination of inhibitors in any suitable ratio. For example, in one embodiment, the method comprises administering three individual inhibitors at a 1:1:1 ratio. In one embodiment, the method comprises administering two individual inhibitors at a 1:1 ratio. However, the method is not limited to any particular ratio. Rather any ratio that is shown to be effective is encompassed.

In one embodiment, the method further comprises administering to the subject at least one additional therapeutic agent. In one embodiment, the therapeutic agent is selected from the group consisting of a chemotherapy, chemotherapeutic agent, radiation therapy, hormonal therapy, PARP inhibitor, immune checkpoint inhibitor, and any combination thereof.

In some embodiments, the method for treating cancer in a subject in need thereof comprises administering an effective amount of a composition comprising at least one compound of Formulae (I) to (V) to the subject prior to, concurrently with, or subsequently to the treatment with a complementary therapy for the cancer, such as surgery, chemotherapy, chemotherapeutic agent, radiation therapy, hormonal therapy, PARP inhibitor, immune checkpoint inhibitor, or any combination thereof.

Examples of PARP inhibitors include, but are not limited to, olaparib, rucaparib, niraparib, talazoparib, veliparib, pamiparib, CEP 9722, E7016, iniparib, 3-aminobenzamide, or any combination thereof.

In some embodiments, immune checkpoint inhibitor is suitable for inhibiting at least one immune checkpoint. In some embodiments, the immune checkpoint is PD1, such as PD-L1 and PD-L2, adenosine A2A receptor (A2AR), cluster of differentiation 276 (CD276), V-set domain-containing T-cell activation inhibitor 1 (VTCN1), B and T lymphocyte attenuator (BTLA), cytotoxic T-lymphocyte-associated protein 4 (CTLA-4), indoleamine 2,3-dioxygenase (IDO), killer-cell immunoglobulin-like receptor (KIR), lymphocyte activation gene-3 (LAG3), nicotinamide adenine dinucleotide phosphate NADPH oxidase isoform 2 (NOX2), T-cell immunoglobulin domain and mucin domain 3 (TIM-3), V-domain Ig suppressor of T cell activation (VISTA), sialic acid-binding immunoglobulin-type lectin 7 (SIGLEC7), or any combination thereof. For example, in one embodiment, the immune checkpoint inhibitor is suitable for inhibiting at least one PD1. In some embodiments, the immune checkpoint inhibitor is suitable for inhibiting at least one PD-L1, PD-L2, or a combination thereof. Thus, in one embodiment, the immune checkpoint inhibitor is an anti-PD-L1 antibody.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

### Administration/Dosage/Formulations

The regimen of administration may affect what constitutes an effective amount. The therapeutic formulations may be administered to the subject either before or after the onset of a disease or infection. Further, several divided dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, (e.g., human), may be carried out using known procedures, at dosages and for periods of time effective to treat the disease or infection in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or infection in the patient. Dosage regimens may be adjusted to provide the optimum therapeutic response. For example, several divided doses may be administered daily. In another example, the dose may be proportionally reduced as indicated by the exigencies of the therapeutic situation. A non-limiting example of an effective dose range for a therapeutic compound of the invention is from about 1 mg/kg to about 5,000 mg/kg of body weight/per day. One of ordinary skill in the art would be able to assess the relevant factors and make the determination regarding the effective amount of the therapeutic compound without undue experimentation.

Actual dosage levels of the active ingredients in the pharmaceutical compositions of this invention may be varied to obtain an amount of the active ingredient that is effective to achieve the desired therapeutic response for a particular patient, composition, and mode of administration, without generating excessive side effects in the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical professional, e.g., physician or veterinarian, having ordinary skill in the art may readily determine and prescribe the effective amount of the pharmaceutical composition required. For example, the physician or veterinarian could start with a dosage of the compound of the invention in the pharmaceutical composition at a level that is lower than the level required to achieve the desired therapeutic effect, and then increase the dosage over time until the desired effect is achieved.

In particular embodiments, it is advantageous to formulate the compound in dosage unit form for ease of administration and uniformity of dosage. “Dosage unit form” as used herein refers to a physically discrete unit containing a predetermined quantity of therapeutic compound calculated to produce the desired therapeutic effect, in association with the required pharmaceutical vehicle. The dosage unit forms of the invention can be selected based upon (a) the unique characteristics of the therapeutic compound and the particular therapeutic effect to be achieved, and (b) the limitations inherent in the art of compounding/formulating such a therapeutic compound for the treatment of a disease or infection in a patient.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), vegetable oils, and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In some embodiments, it is useful to include isotonic agents, for example, sugars, sodium chloride, or polyalcohols such as mannitol and sorbitol, in the composition. Prolonged absorption of the injectable compositions can be achieved by including in the composition an agent which delays absorption, for example, aluminum monostearate or gelatin. In one embodiment, the pharmaceutically acceptable carrier is DMSO, alone or in combination with other carriers.

The therapeutically effective amount or dose of a compound of the present invention depends on the age, sex and weight of the patient, the current medical condition of the patient and the severity of the disease or infection in the patient being treated. The skilled artisan is able to determine appropriate doses depending on these and other factors.

The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

Doses of the compound of the invention for administration may be in the range of from about 1 μg to about 10,000 mg, from about 20 μg to about 9,500 mg, from about 40 μg to about 9,000 mg, from about 75 μg to about 8,500 mg, from about 150 μg to about 7,500 mg, from about 200 μg to about 7,000 mg, from about 3050 μg to about 6,000 mg, from about 500 μg to about 5,000 mg, from about 750 μg to about 4,000 mg, from about 1 mg to about 3,000 mg, from about 10 mg to about 2,500 mg, from about 20 mg to about 2,000 mg, from about 25 mg to about 1,500 mg, from about 30 mg to about 1,000 mg, from about 40 mg to about 900 mg, from about 50 mg to about 800 mg, from about 60 mg to about 750 mg, from about 70 mg to about 600 mg, from about 80 mg to about 500 mg, and any and all whole or partial increments therebetween.

In some embodiments, the dose of a compound of the invention is from about 1 mg to about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, the dosage of a second compound as described elsewhere herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

In one embodiment, the compositions of the invention are administered to the patient from about one to about five times per day or more. In various embodiments, the compositions of the invention are administered to the patient, 1-7 times per day, 1-7 times every two days, 1-7 times every 3 days, 1-7 times every week, 1-7 times every two weeks, and 1-7 times per month. It is readily apparent to one skilled in the art that the frequency of administration of the various combination compositions of the invention will vary from individual to individual depending on many factors including, but not limited to, age, the disease or disorder to be treated, the severity of the disease or disorder to be treated, gender, overall health, and other factors. Thus, the invention should not be construed to be limited to any particular dosing regime and the precise dosage and composition to be administered to any patient is determined by the medical professional taking all other factors about the patient into account.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, may be reduced to a level at which the improved disease is retained. In some embodiments, a patient may require intermittent treatment on a long-term basis, or upon any recurrence of the disease or disorder.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In one embodiment, the present invention is directed to a packaged pharmaceutical composition comprising a container holding a therapeutically effective amount of a compound of the invention, alone or in combination with a second pharmaceutical agent; and instructions for using the compound to treat or prevent a disease or infection in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for parenteral administration and the like. It should be understood that the formulations and compositions that would be useful in the present invention are not limited to the particular formulations and compositions that are described herein.

### Oral Administration

For oral administration, suitable forms include tablets, dragees, liquids, drops, suppositories, or capsules, caplets and gelcaps. The compositions formulated for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica);

disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation involves the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution. U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of G-protein receptor-related diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release refers to a drug formulation that provides for gradual release of a drug over an extended period of time, and that may, although not necessarily, result in substantially constant blood levels of a drug over an extended time period. The period of time may be as long as a day, a week, or a month or more and should be a release which is longer that the same amount of agent administered in bolus form. The term delayed release is used herein in its conventional sense to refer to a drug formulation that provides for an initial release of the drug after some delay following drug administration and that mat, although not necessarily, includes a delay of from about 10 minutes up to about 12 hours.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term pulsatile release refers to a drug formulation that provides release of the drug in such a way as to produce pulsed plasma profiles of the drug after drug administration.

The term immediate release refers to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art recognize, or are able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out certain embodiments of the present invention, and are not to be construed as limiting in any way the remainder of the disclosure.

### Example 1: Novel Small Molecule Inhibitor for Ovarian Cancer Therapy—in Silico Screening Identified a Novel Small Molecule Inhibitor that Counteracts PARP Inhibitor Resistance in Ovarian Cancer

To overcome the limitation of PARP inhibitors, the present studies have undertaken the discovery and development of small molecule inhibitors of HR repair aimed to the treatment of BRCA-wild type or HR-proficient EOC. Previously disclosed triapine is a small molecule inhibitor of ribonucleotide reductase (RNR) and 1,000 times more potent than the clinically used RNR inhibitor hydroxyurea (Finch R A et al., 2000, Biochem Pharmacol, 59:983-991; Finch R A et al., 1999, Adv Enzyme Regul, 39:3-12). RNR is a heteromeric enzyme consisting of R2 and R1 subunits during the S phase of the cell cycle (Lin Z P et al., 2004, J Biol Chem, 279:27030-27038; Tanaka H et al., 2000, Nature, 404:42-49). RNR catalyzes the rate-limiting step in the conversion of ribonucleoside diphosphates (NDPs) into corresponding deoxyribonucleoside diphosphates (dNDPs), the immediate precursors of deoxyribonucleoside triphosphates (dNTPs) essential for DNA replication and repair (Thelander L et al., 1979, Annu Rev Biochem, 48:133-158). Triapine quenched the tyrosyl radical in the R2 subunit of RNR, thereby leading to enzymatic inactivation (Sartorelli A C et al., 1976, Adv Enzyme Regul, 15:117-139; Shao J et al., 2006, Mol Cancer Ther, 5:586-592). As a result, treatment with triapine promptly caused depletion of dNTPs and stalling of replicative DNA synthesis (Lin Z P et al., 2011, Mol Pharmacol, 80:1000-1012; Cory J G et al., 1995, Adv Enzyme Regul., 35:55-68).

Preclinical and clinical studies have demonstrated that triapine works effectively as chemo- and radio-sensitizer to augment the anticancer activity of DNA damaging agents and radiation (Finch R A et al., 2000, Biochem Pharmacol, 59:983-991; Barker C A et al., 2006, Clin Cancer Res, 12:2912-2918; Lin Z P et al., 2014, Molecular Cancer Res, 12:381-393; Ratner E S et al., 2016, Br J Cancer, 114:777-786). With more than 80% clinical response rates in phase I/II studies (Kunos C A et al., 2013, Gynecol Oncol, 130:75-80; Kunos C A et al., 2010, Clin Cancer Res, 16:1298-1306; Kunos C A et al., 2014, Front Oncol, 4:184), Triapine is currently being studied in combination with cisplatin and radiation under a randomized phase III trial (NCT02466971) and a phase I trial (NCT02595879) in cervical and virginal cancers (Kunos C A et al., 2010, Clin Cancer Res, 16:1298-1306; Kunos C A et al., 2019, Front Oncol, 9:1067). Triapine was well tolerated in patients and its side effects were generally manageable. The side effects of triapine included methemoglobinemia and dyspnea due to its strong iron-chelation property (Kunos C A et al., 2012, Future Oncol, 8:145-150).

The mechanistic studies have demonstrated that triapine inhibited CDK activity, abrogated CtIP-mediated DSB end resection, suppressed HR repair, and sensitized BRCA-wild type EOC to PARP inhibitors and platinum drugs in cell-based assays and tumor xenograft mouse models (Lin Z P et al., 2014, Molecular Cancer Res, 12:381-393; Ratner E S et al., 2016, Br J Cancer, 114:777-786). In keeping with these finding, independent studies also provided evidence that triapine blocked CtIP-dependent end resection of DBSs (Shou J et al., 2018, Mol Cell, 71:498-509), and inhibition of RNR by hydroxyurea suppresses HR repair (Burkhalter M D et al., 2009, DNA Repair (Amst), 8:1258-1263). It was also substantiated that depletion of the R2 subunit of RNR by siRNA suppressed the repair of endonuclease-induced DSBs by HR (Lin Z P et al., 2014, Molecular Cancer Res, 12:381-393; Ratner E S et al., 2016, Br J Cancer, 114:777-786). Furthermore, it was shown that the combination of triapine, olaparib, and cediranib effectively curbed subcutaneous growth and peritoneal progression of BRCA-wild type EOC xenografts and extended the survival time of mice (Lin Z P et al., 2018, PLOS One, 13:e0207399). Cediranib is a small molecule inhibitor of vascular endothelial growth factor (VEGF) receptor tyrosine kinases included in the combination to enhance the efficacy of olaparib and triapine.

The present study sought to discover the next-generation RNR inhibitors to circumvent some pharmacodynamic and pharmacokinetic issues of triapine. Triapine is known to exhibit a short half-life in plasma (about 2 hr) and hematological side effects linked to its iron-chelating property. Using the approach of in silico screening for hit enrichment and experimental validation of inhibitory potency, 2-[(4-{4-allyl-5-[(3-chlorobenzyl)thio]-4H-1,2,4-triazol-3-yl}-1-piperidinyl)methyl]-1,3-benzothiazole named DB4, a putative small molecule inhibitor of RNR that abrogated HR repair and sensitized BRCA-wild type or HR repair-proficient EOC, was identified. This discovery provided a potential alternative to triapine and a new class of small molecule inhibitors for future drug design and development for ovarian cancer therapy.

### In Silico Screening of a Compound Library for Docking the Triapine-Binding Pocket on the R2 Subunit of RNR

It has been postulated that triapine binds to a surface pocket of the R2 subunit of RNR (Zhou B et al., 2013, Cancer Res, 73:6484-6493; Popovic-Bijelic A et al., 2011, J Inorg Biochem, 105:1422-1431). This putative triapine-binding pocket positioned in a close proximity of the di-ferric iron center and the tyrosyl-radical residue critical for the reduction activity of RNR (Popovic-Bijelic A et al., 2011, J Inorg Biochem, 105:1422-1431). It also lied in the interface between R2 and R1 subunits of the RNR complex (Zhou B et al., 2013, Cancer Res, 73:6484-6493). Given its strong inhibitory activity toward RNR, triapine reportedly bound to this binding pocket of the R2 subunit to facilitate the inhibition of the reduction of ribonucleotide diphosphates (NDPs).

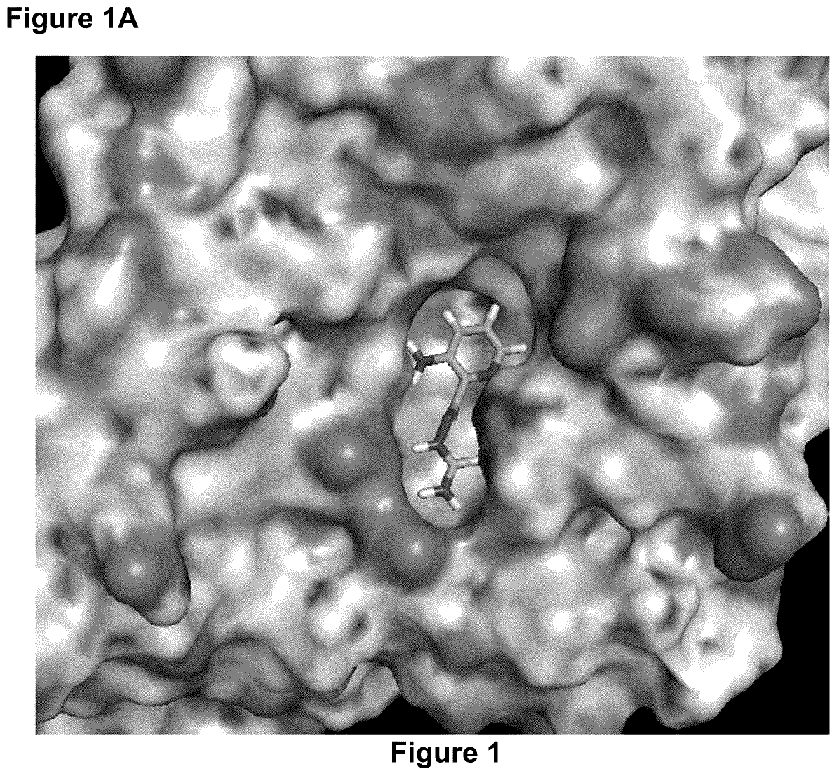

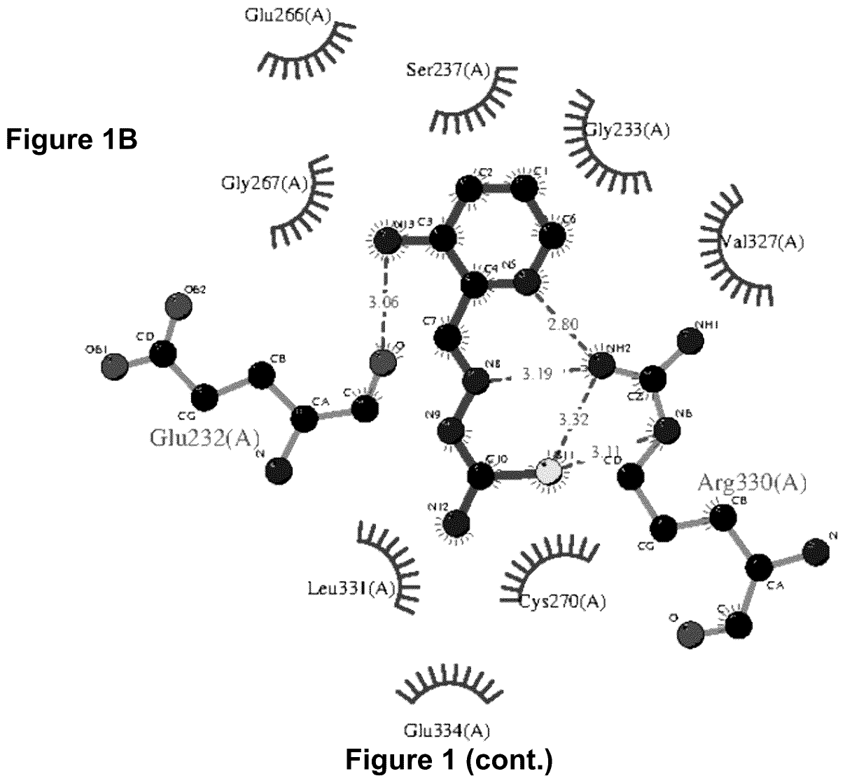

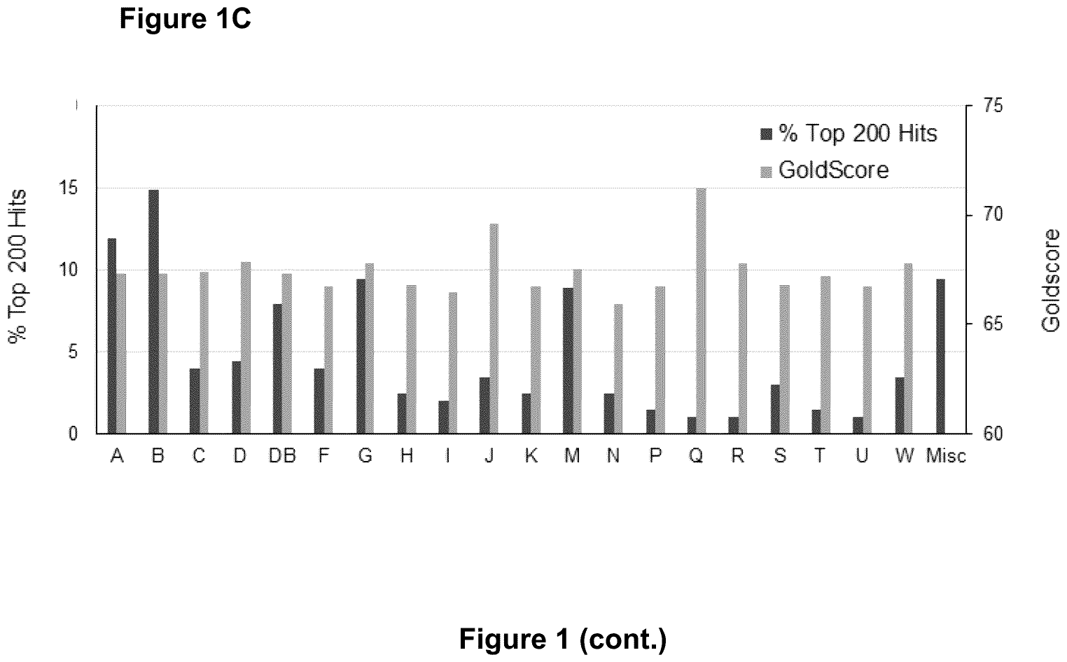

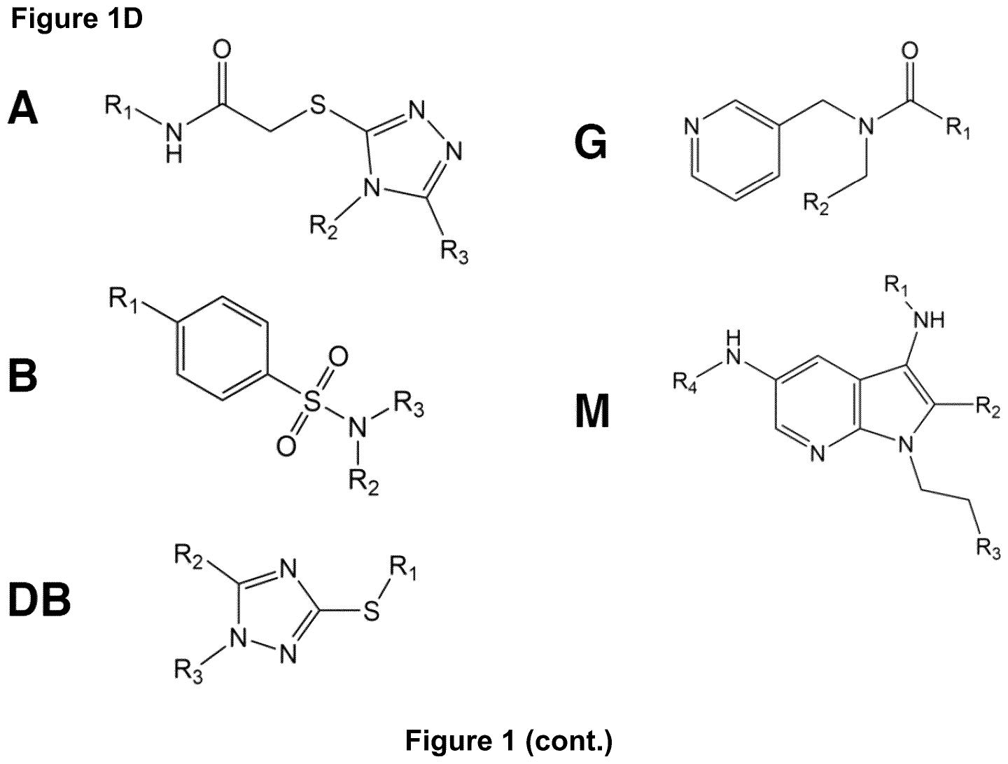

The docking of triapine into the triapine-binding pocket were performed using the GOLD docking program (Cambridge Crystallographic Data Centre) and the rendering/modeling of molecular interactions using the PyMOL (Schrödinger, Inc.) and the LigPlot+ (The European Bioinformatics Institute) programs. The results showed that triapine forms hydrogen bonds and several hydrophobic interactions with the triapine-binding pocket (FIG. 1A and FIG. 1B).

In silico screening of two subsets of the compound library (Chembridge) consisting of approximate 200,000 compounds were conducted using the GOLD program. The crystal structure of the R2 subunit of RNR was used for local docking of compounds into the triapine-binding pocket (FIG. 1A and FIG. 1B). The binding site was defined as the surface cavity centering Gly233 of the R2 subunit. Compounds were docked, scored, and ranked according to their GOLDscores. Hit clustering of 200 top-ranking compounds was performed based on structural and moiety similarities. Five distinct pharmacophores, each of which constituted 7-15% of those 200 compounds, were identified (FIG. 1C and FIG. 1D). Depending primarily on the commercial availability of these hits, total 25 compounds consisting of 3 to 9 compounds per pharmacophore group were arbitrarily chosen and acquired from Chembridge.

### Secondary Screening of in Silico Hits by Cell-Based Assays

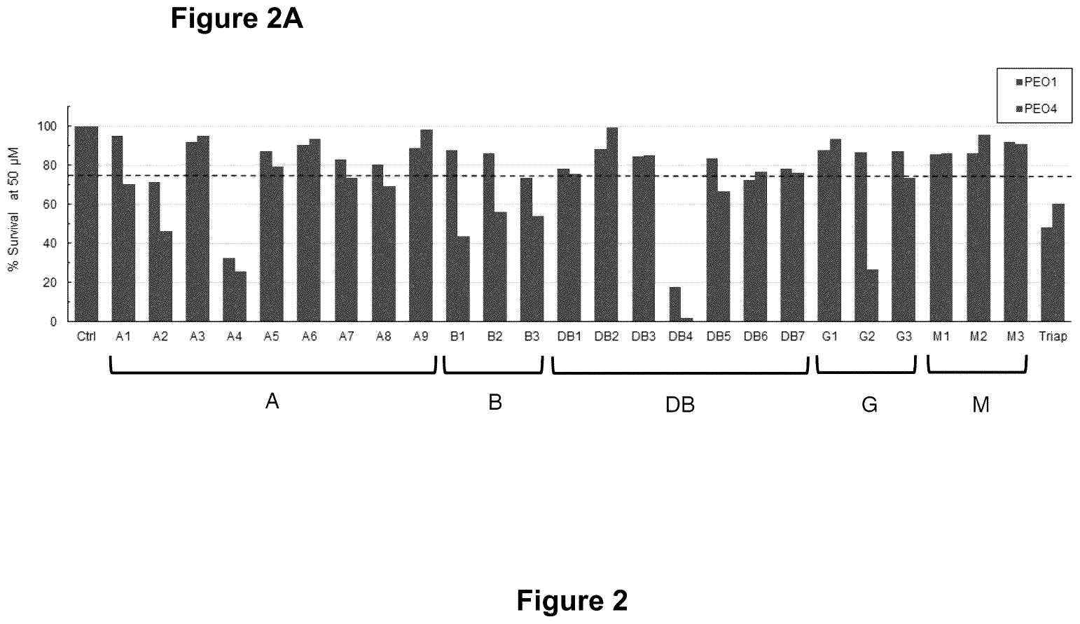

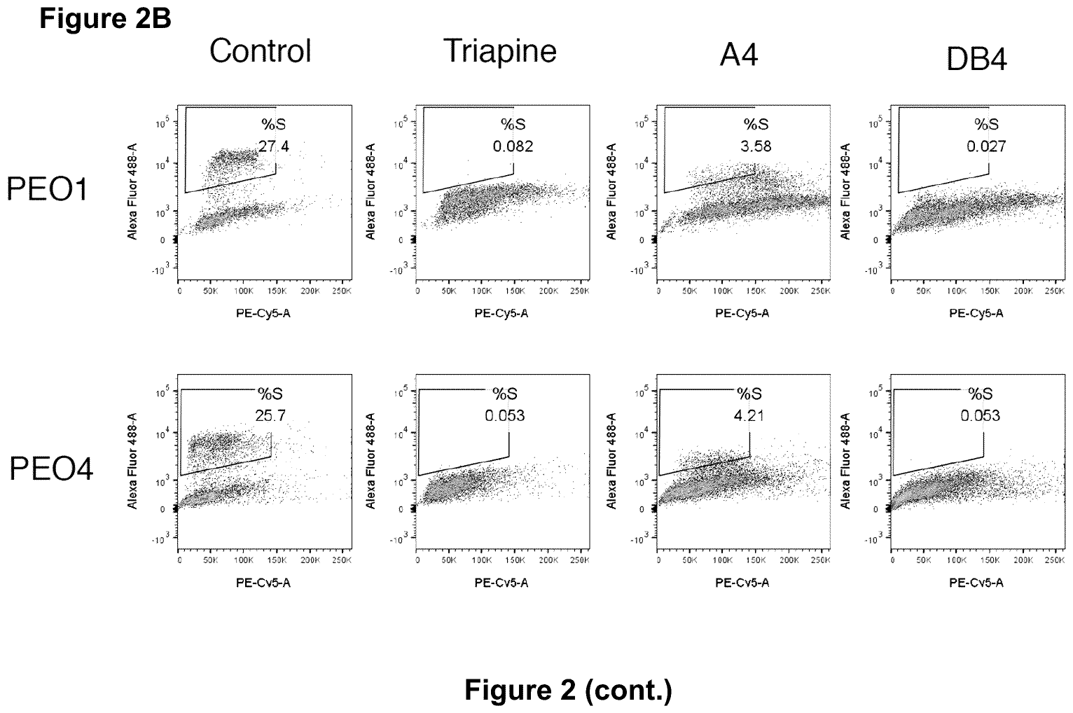

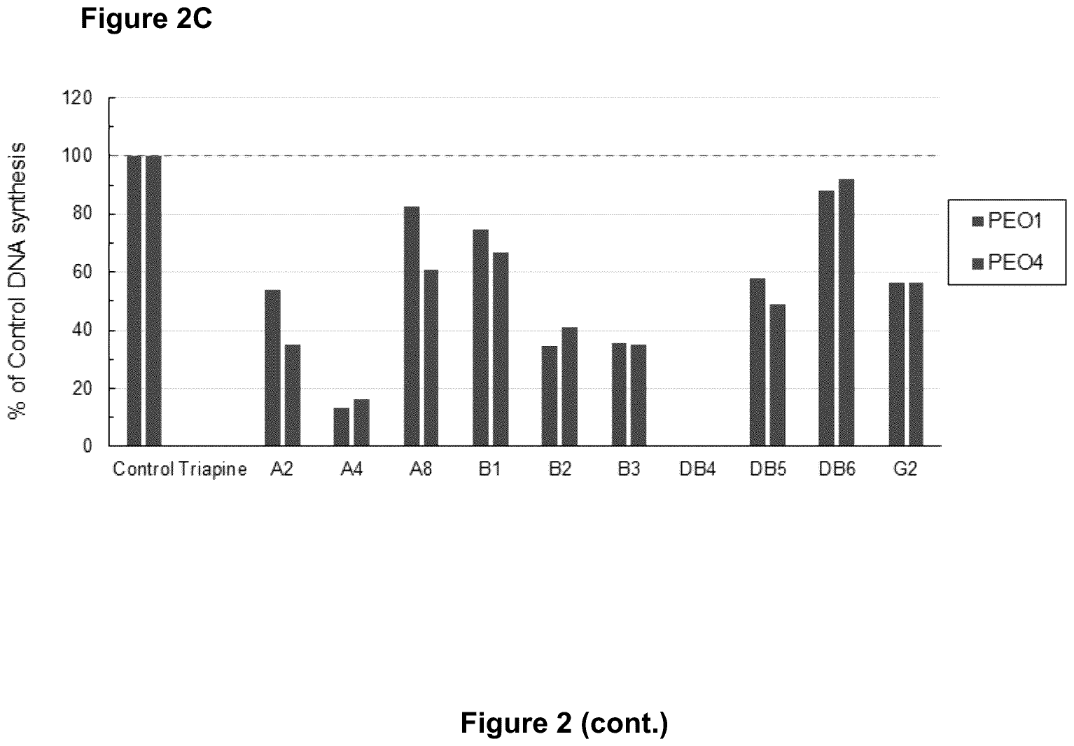

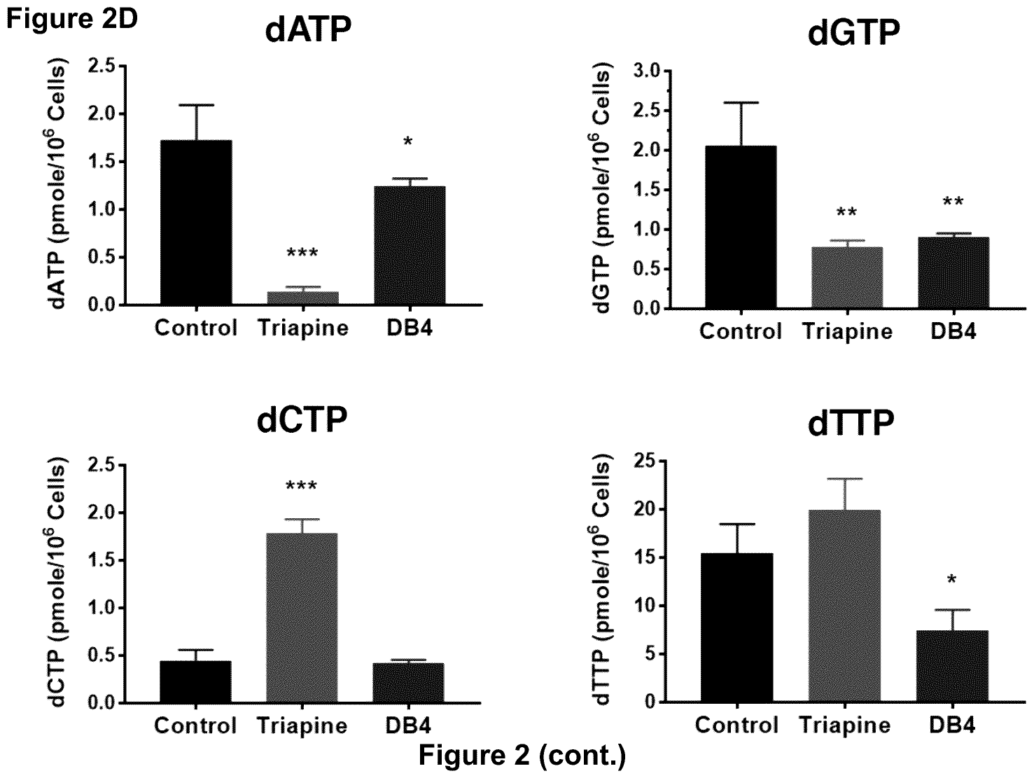

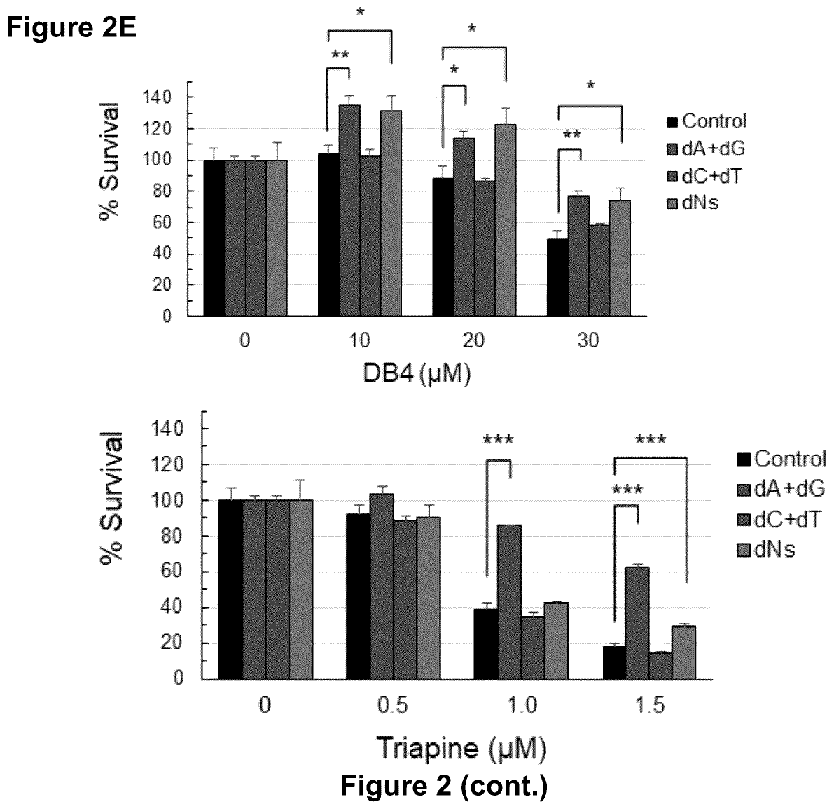

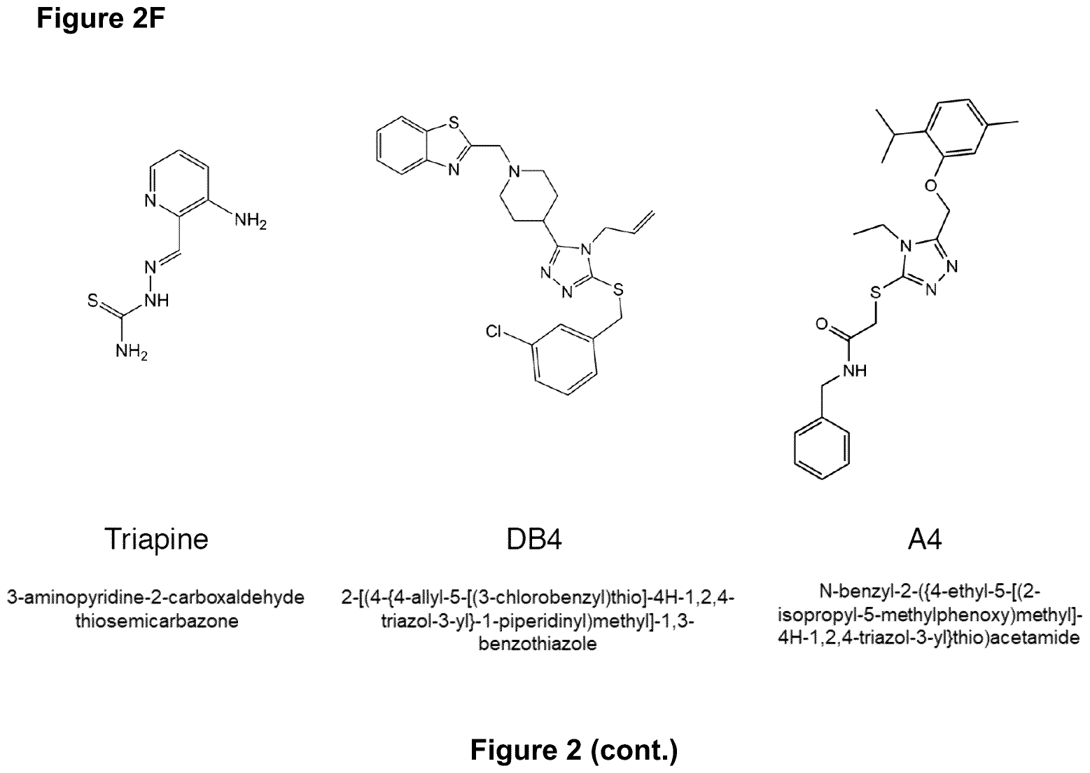

Next, 25 hit compounds were screened for their ability to inhibit cell proliferation at 50 μM using MTS cytotoxicity assay (FIG. 2A). Judged by causing equal to more than 25% inhibition of either PEO1 or PEO4 cells, 10 compounds were advanced and evaluated for the inhibition of DNA synthesis. Using the flow cytometric EdU incorporation assay, the most active compound DB4 that caused nearly complete inhibition (>99%) of DNA synthesis in a manner similar to triapine in PEO1 and PEO4 cells was identified (FIG. 2B and FIG. 2C).

Furthermore, the dNTP measurements were performed to evaluate the inhibitory effects of DB4 on RNR compared with triapine. DB4 caused significant decreases in the levels of dATP, dGTP, and dTTP. Consistent with the previous findings (Lin Z P et al., 2004, J Biol Chem, 279:27030-27038; Lin Z P et al., 2011, Mol Pharmacol, 80:1000-1012; Lin Z P et al., 2007, Biochem Pharmacol, 73:760-772), triapine produced a significant and pronounced decreases in the levels of dATP and dGTP while causing a marked increase in dCTP and somewhat elevated dTTP (FIG. 2D). The increases in dCTP and dTTP levels caused by triapine are attributable to the dominance of the pyrimidine salvage pathway in mammalian cells (Löffler M et al., 2005, Trends Mol Med, 11:430-437).

To determine whether exogenous sources of dNTPs bypass the inhibitory effects of DB4 and triapine on RNR, deoxyribonucleosides (dNs) were supplemented in the medium in the presence of DB4 or triapine. dNs is transported into cells and subsequently converted to dNDPs and then dNTPs for DNA synthesis (Lagergren J et al., 1987, Biochem Pharmacol, 36:2985-2991). The survival of cells was determined by the MTS cytotoxicity assay as a readout. The result showed that addition of dA plus dG, or all dNs, partially reversed the inhibitory effects of DB4 and triapine on cell survival (FIG. 2E).

In contrast, addition of dC plus dT had no effects and somewhat counteracted dA plus dG on reversing the effects of triapine, indicating that a delicately balance among individual dNTP levels was required for faithful DNA synthesis. Nevertheless, these findings were consistent with the results of dNTP measurements (FIG. 2D), in which DB4 and triapine caused a predominant decrease in dATP and dGTP levels. Because DB4 consistently led to suppression of cell proliferation, DNA replication, and dNTP synthesis indicative of RNR inhibition, DB4 was chosen for the following studies. DB4 is structurally unrelated to triapine and had no apparent features of known iron chelating compounds (FIG. 2F). A4 was the second active compound that loosely share structural similarity with DB4.

### Structural Activity Relationship (SAR) Between DB4 and the Triapine-Binding Pocket

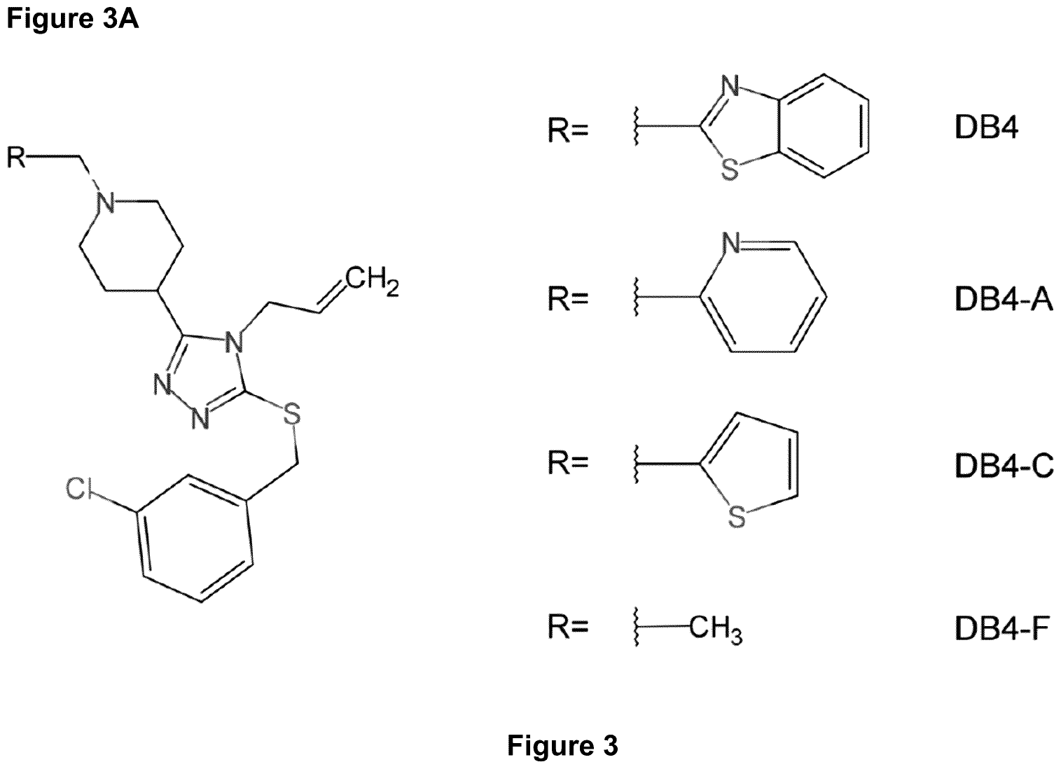

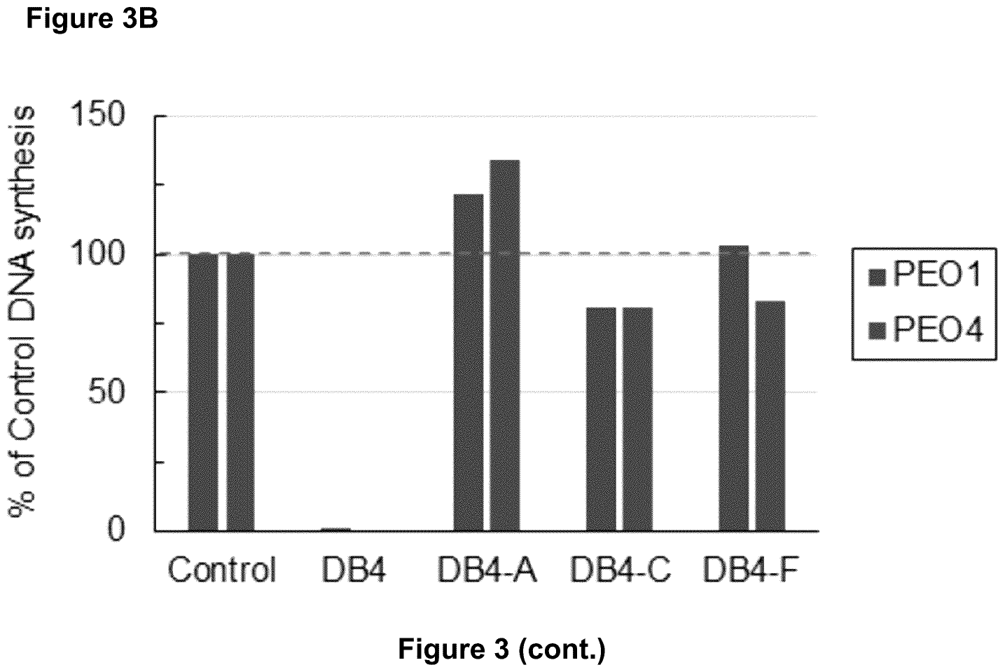

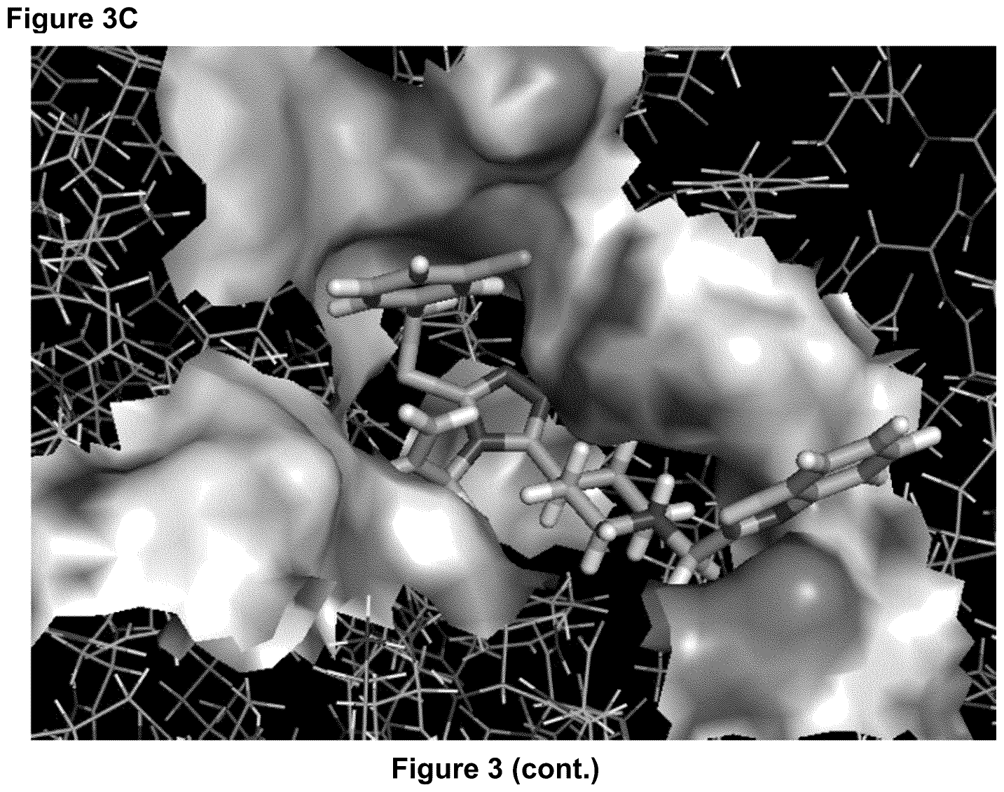

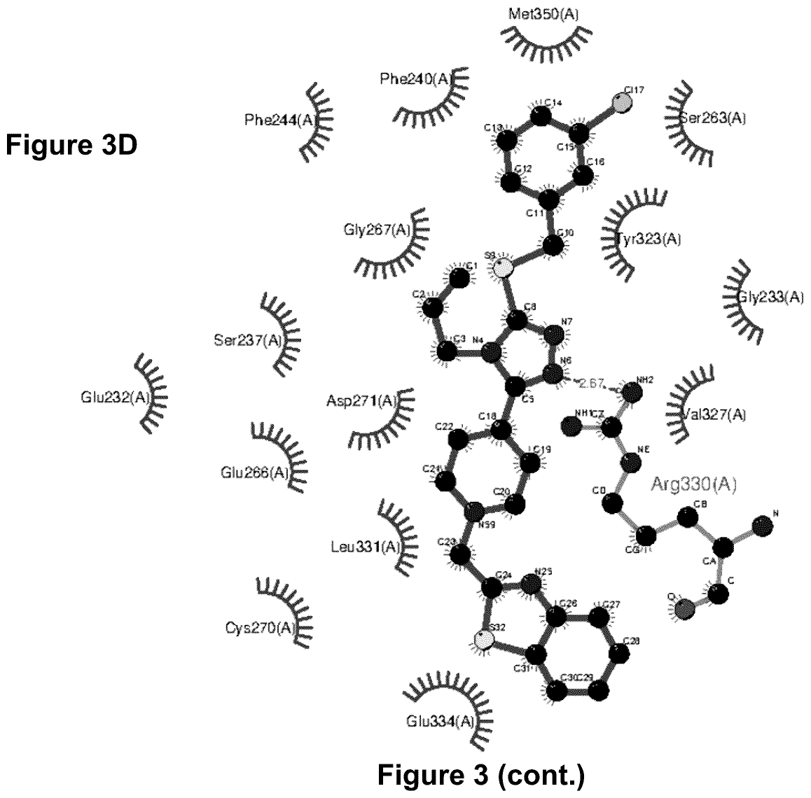

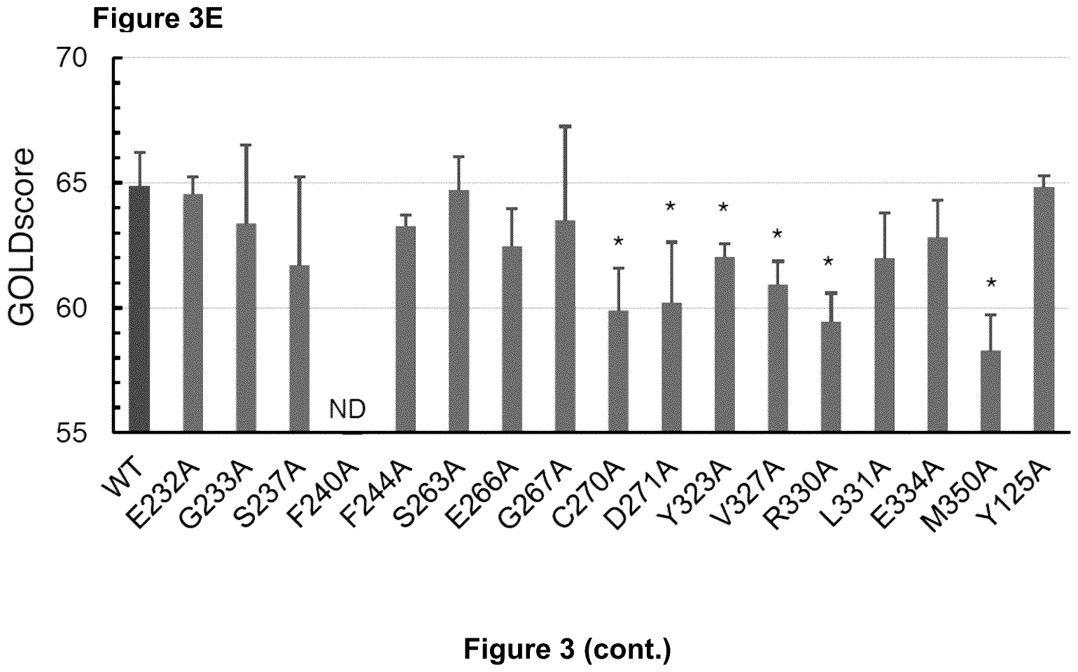

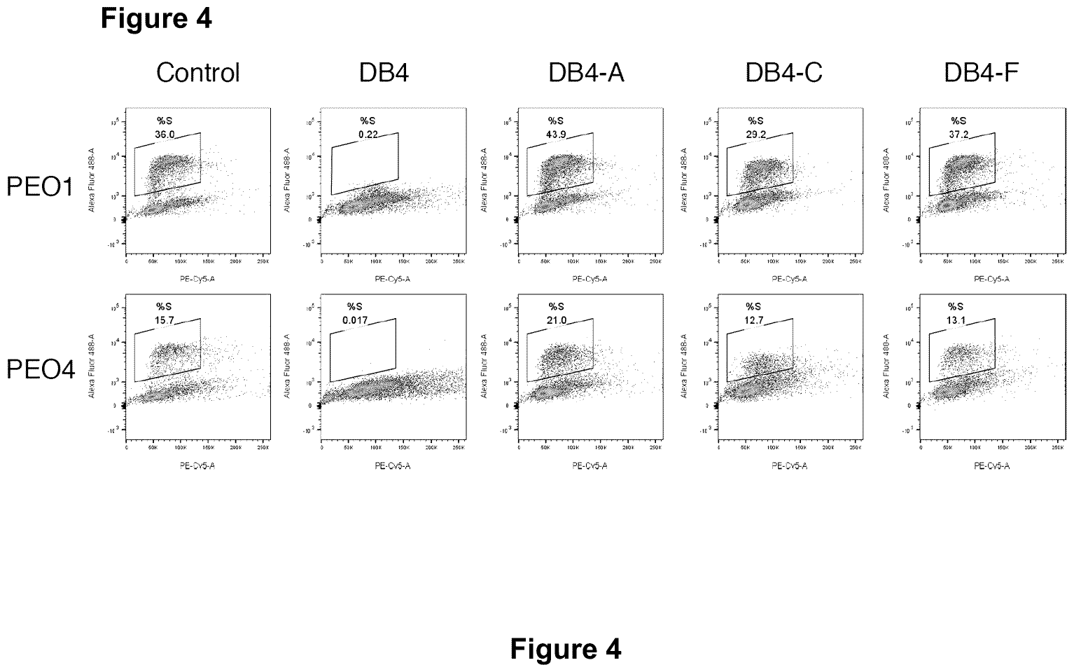

Similarity search was conducted for structural analogs of DB4 from Chembridge's compound libraries. Three compounds of DB4 analogs were obtained for evaluating their ability to inhibit DNA synthesis compared with DB4 (FIG. 3A). These compounds retained the core structure of DB4 with its methyl-benzothiazole group being substituted with methyl-pyridine, methyl-thiophene, or propyl group, as DB4-A, DB4-C, and DB4-F, respectively. PEO1 and PEO4 cells were treated with these compounds for 24 hr and DNA synthesis inhibition was determined by the flow cytometric EdU incorporation assay as described above. All DB4 analogs did not apparently inhibit DNA synthesis as opposed to DB4 in both cell lines (FIG. 3B and FIG. 4).

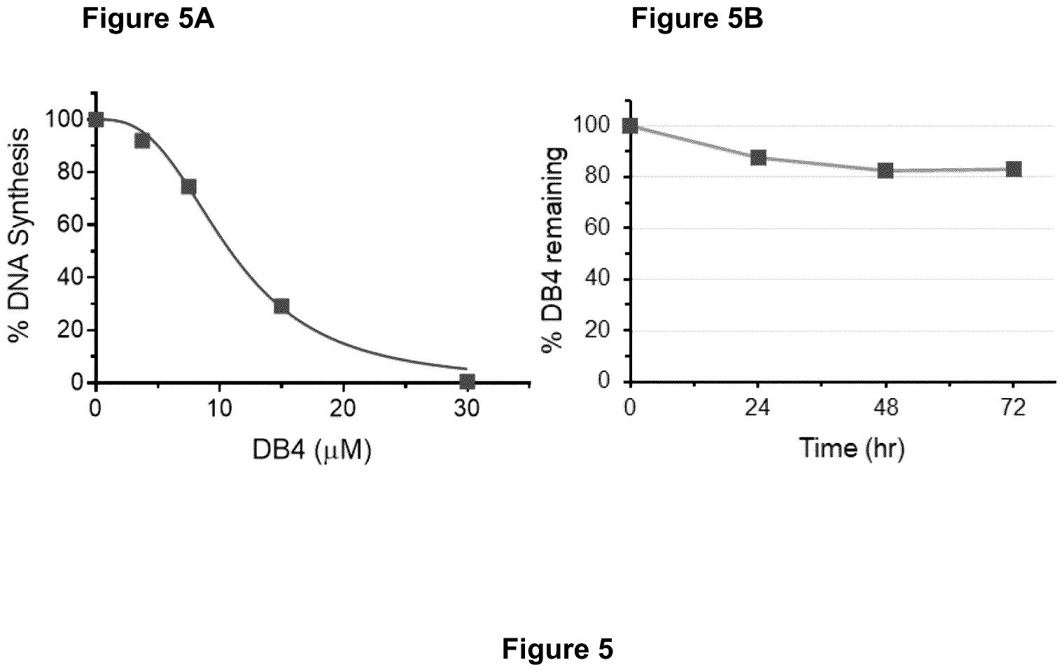

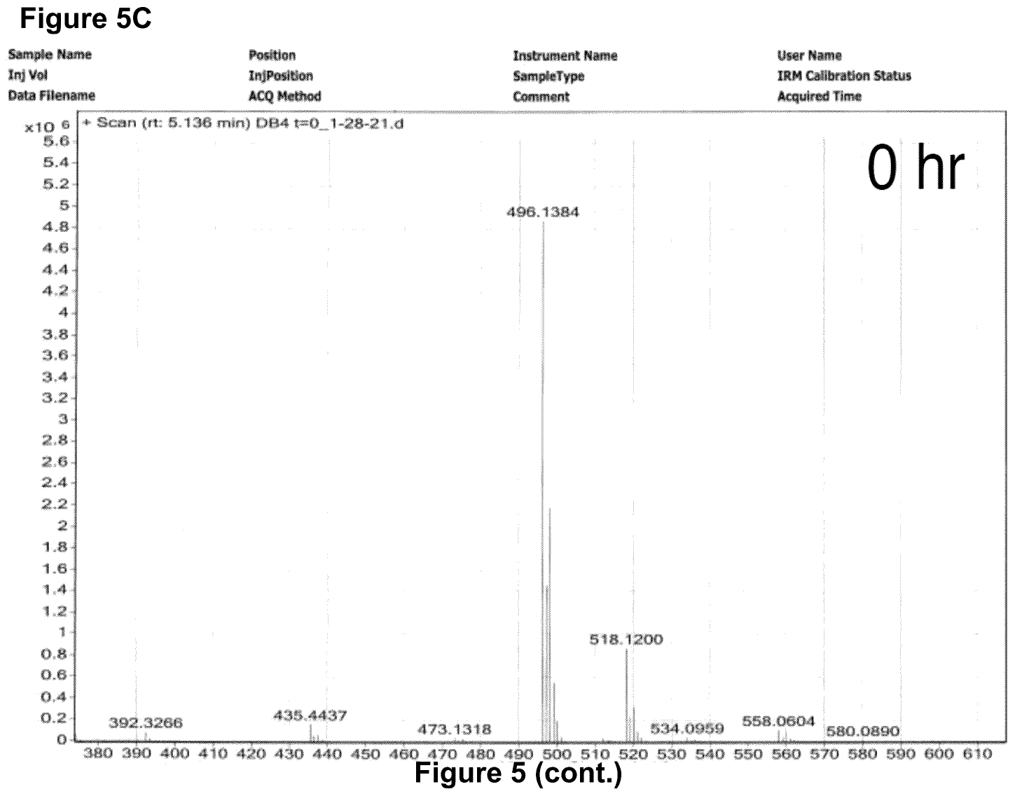

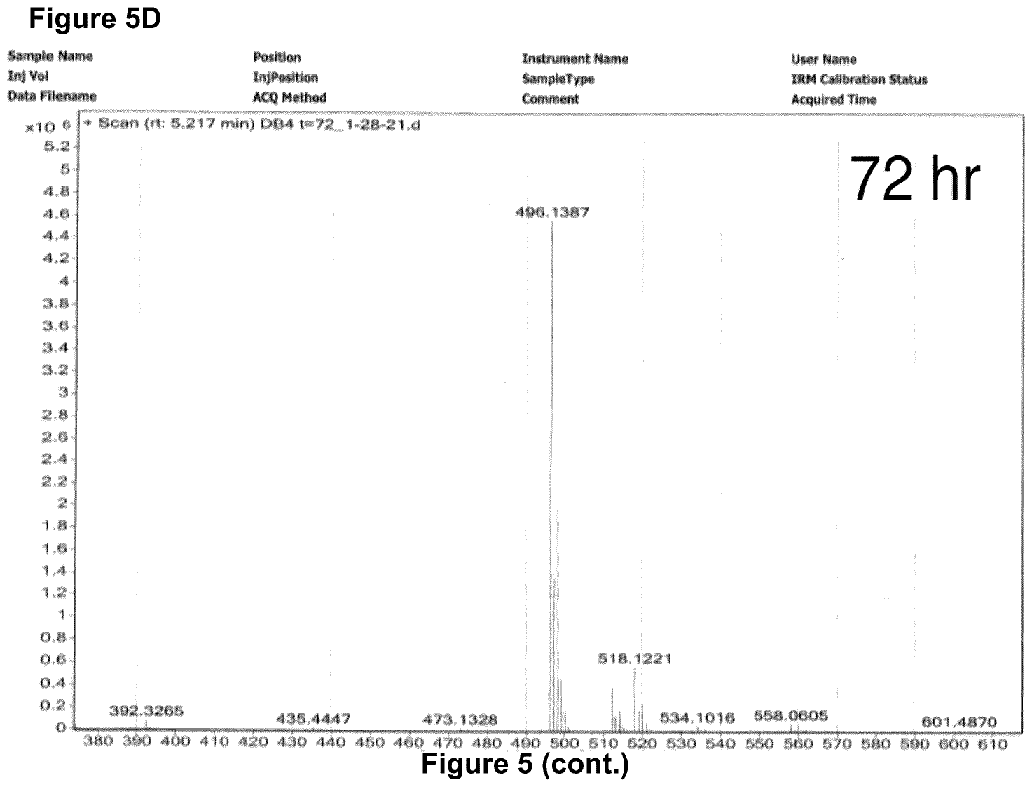

To ensure that DB4 remained chemically stable, the time course study was performed by determining the potency of DB4 following a pre-incubation in the cell-free medium at 37° C. for up to 72 hr. Cells were treated for 24 hr with the medium containing pre-incubated DB4 and DNA synthesis was determined by the EdU incorporation assay. The results showed that pre-incubated DB4 up to 72 hr was more than 82% active to inhibit DNA synthesis in PEO4 cells (FIG. 5A and FIG. 5B). Furthermore, the mass spectrometry analysis substantiates that the level of DB4 at 72 hr remained similar to that of the 0 hr control (FIG. 5C and FIG. 5D).

To gain the insights into the molecular interactions between DB4 and the triapine-binding pocket, re-docking of DB4 were performed as described in FIG. 1 and its potential binding pose was modeled. The results showed that DB4 binds to the triapine-binding pocket through a hydrogen bond and an array of hydrophobic interactions (FIG. 3C, FIG. 3D, and Table 1). Similar to triapine, DB4 formed a hydrogen bond with Arg330 of the binding pocket but lacks a hydrogen bond with Glu232. DB4 also shared all eight hydrophobic interactions with triapine and possessed additional seven hydrophobic interactions with the binding pocket.

TABLE 1
Comparison of putative molecular interactions of the
triapine-binding pocket with triapine and DB4.

| Interaction Type | Triapine | DB4 |
|---|---|---|
| Hydrogen Bond | Glu232 | |
| | Arg330 | Arg330 |
| Hydrophobic | | Glu232 |
| Interaction | Gly233 | Gly233 |
| | Ser237 | Ser237 |
| | | Phe240 |
| | | Phe244 |
| | | Ser263 |
| | Glu266 | Glu266 |
| | Gly267 | Gly267 |
| | Cys270 | Cys270 |
| | | Asp271 |
| | | Tyr323 |
| | Val327 | Val327 |
| | Glu334 | Glu334 |
| | Leu331 | Leu331 |
| | | Met350 |

To interrogate the importance of in silico interaction between DB4 and the triapine-binding pocket, mutations were engineered to disrupt key binding interactions implicated by the docking model. We performed in silico mutagenesis of 16 amino acid residues (FIG. 3C, FIG. 3D, and Table 1) putatively interacting with DB4 in the triapine-binding pocket of the R2 subunit. Using the GOLD program, DB4 was re-docked into the binding pocket of each of mutated R2 subunits. The GOLDScore of three top-ranking docking poses (similar to that of FIG. 3C and FIG. 3D) were averaged and compared with that of the non-mutated control and a mutated negative control (Y125, a residue remote from the binding pocket). The results showed that in silico mutations in 7 out of 16 amino acid residues led to a significant decrease in the GOLDScore (FIG. 3E), indicative of a reduced ability of DB4 to interact with the triapine-binding packet.

### Comparison of DB4 and Triapine in Modulating Cell Cycle Distribution, CDK2 Activity, and DSB End Resection

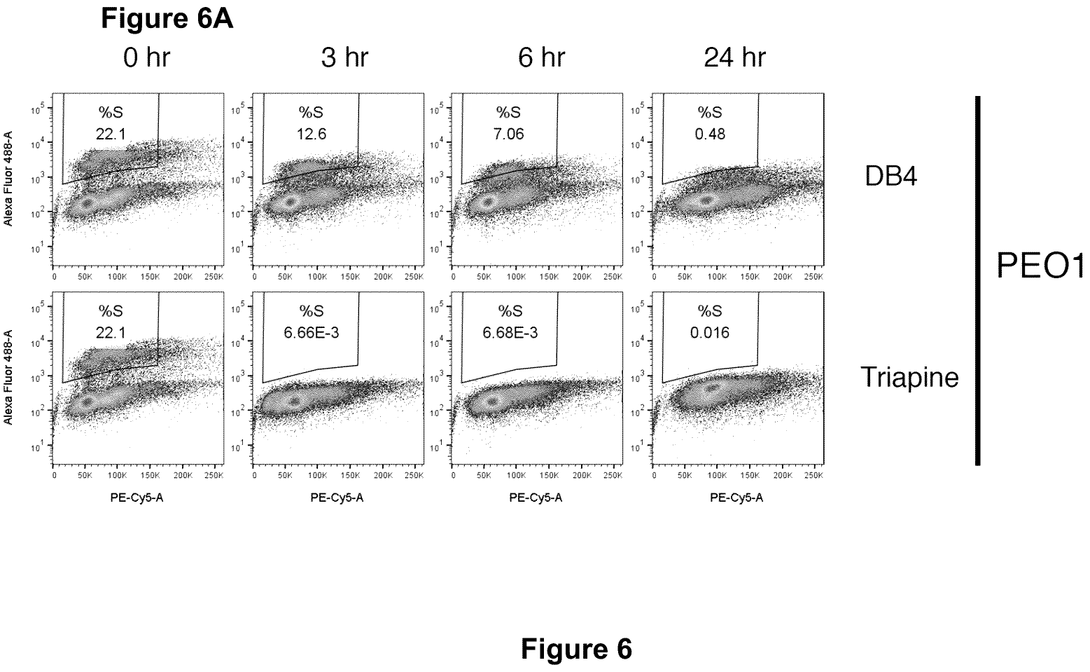

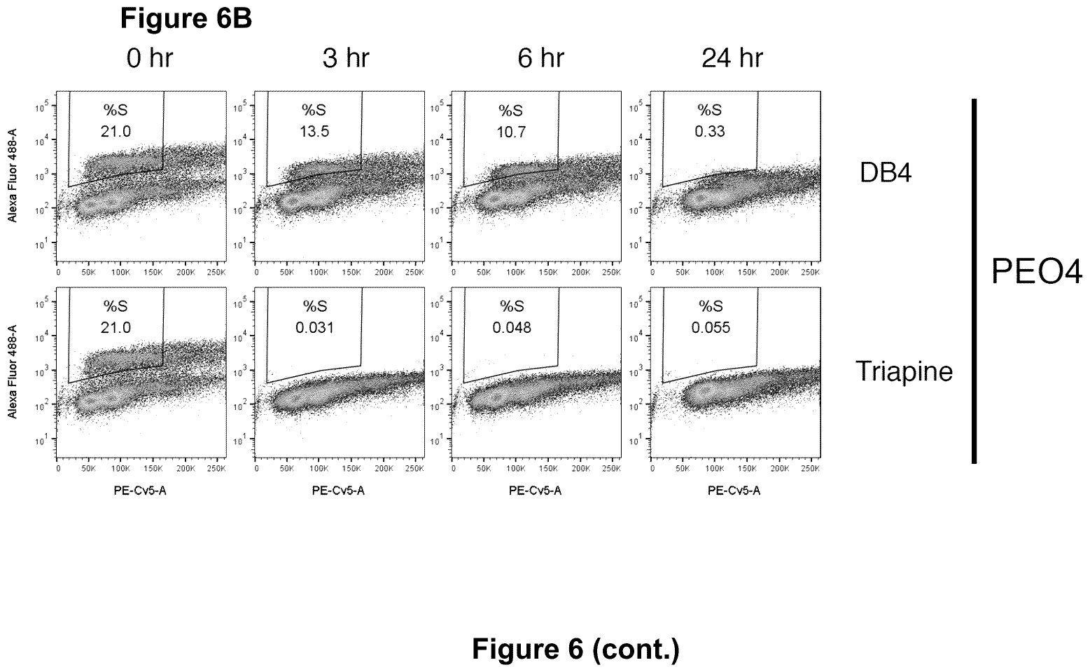

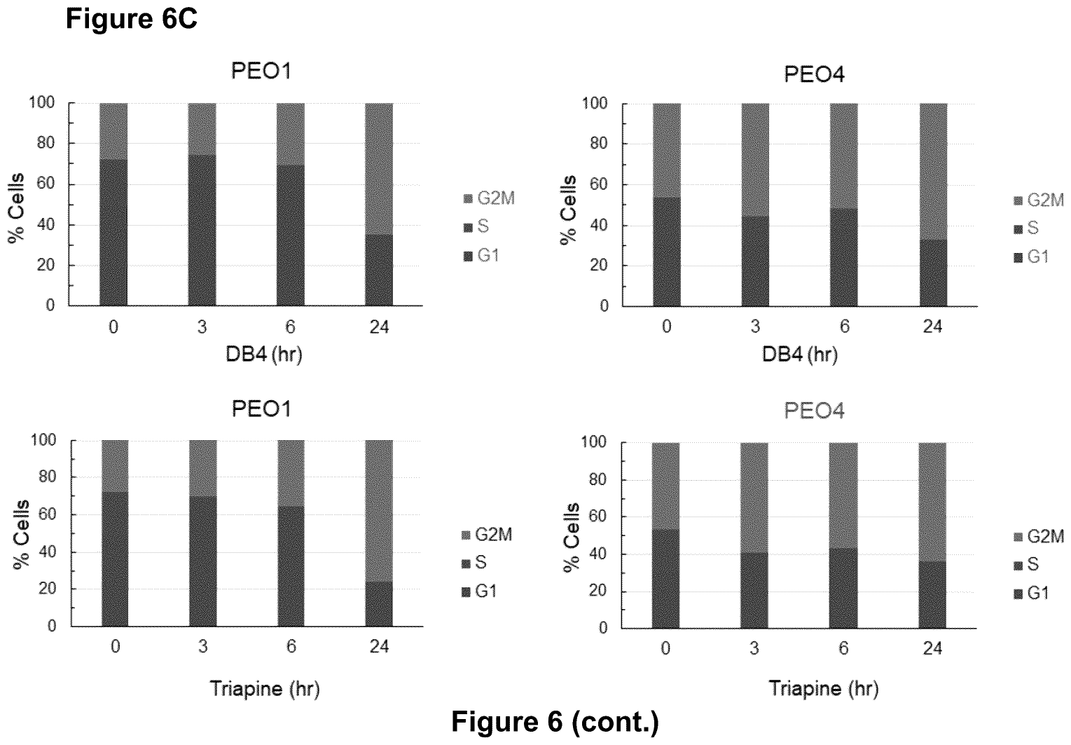

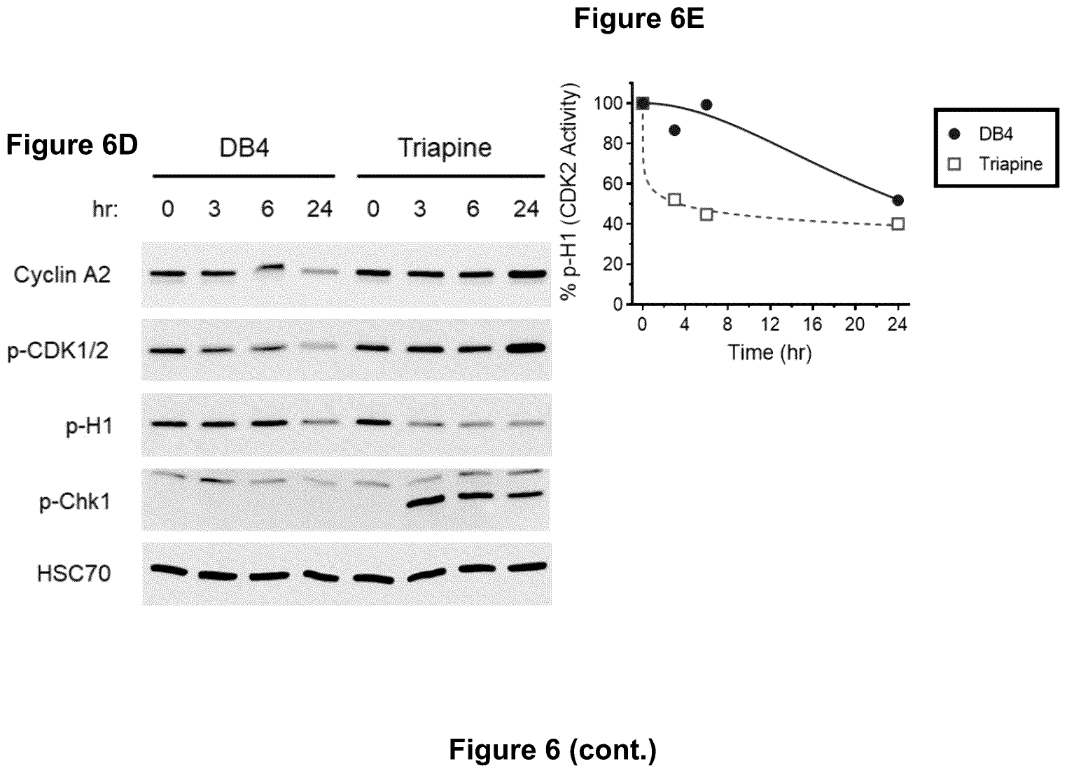

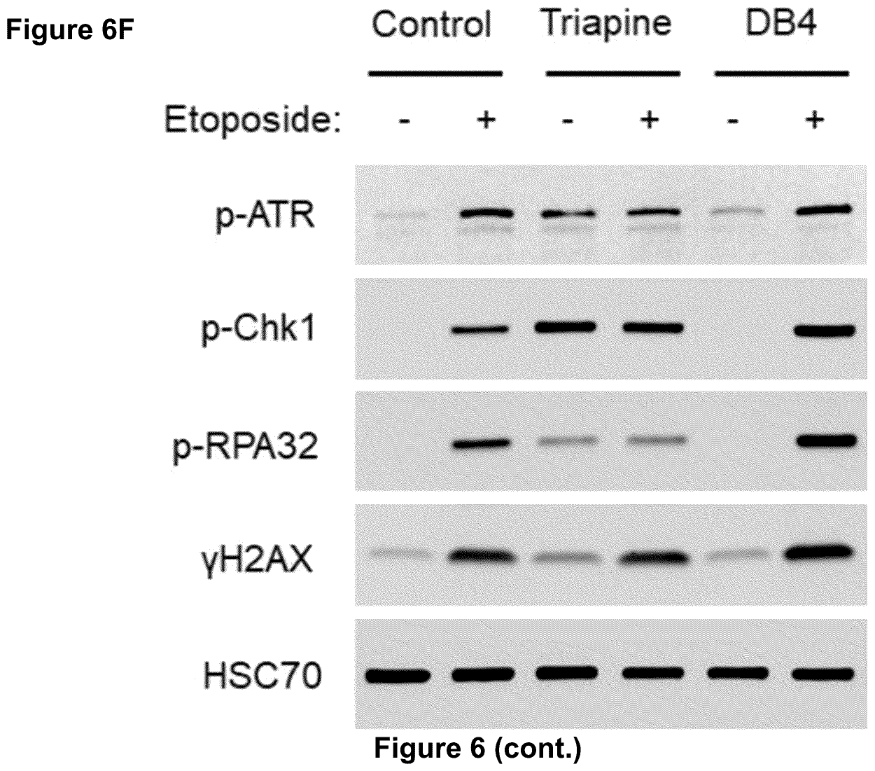

A time course study was performed to comparatively assess how DB4 and triapine affect DNA synthesis and cell cycle in PEO1 and PEO4 cells. Both cell lines displayed a time-dependent inhibition of DNA synthesis by DB4 or triapine (FIG. 6A and FIG. 6B). Triapine caused a prompt reduction in EdU-positive cells, detected at 3 hr and remained very low at 24 hr. In contrast, DB4 led to a gradual decline in the EdU-positive cell population, sustaining a partial reduction at 3 hr and 6 hr, and only becoming very low at 24 hr. Furthermore, the analysis of cell cycle distribution shown in FIG. 6A and FIG. 6B was performed. Both DB4 and triapine caused an expansion of the G2/M population, presumably stemmed from a reduction in G1 and S phase populations in PEO1 cells (FIG. 6C). Similar phenomenon was also found in PEO4 cells, except that the G1 population remained relatively unchanged (30-35%). The S phase population of PEO4 cells appeared to incorporate to the G2/M population by 24 hr.

Additional studies focused on determining how DB4 inhibited CDK2 activity in comparison with triapine. In PEO4 cells, DB4 downregulated the levels of cyclin A2 and CDK2 phosphorylation most prominently at 24 hr (FIG. 6D). In contrast, triapine increased the levels of cyclin A2 and CDK2 phosphorylation evidently at 24 hr. Because CDK2 phosphorylation is inhibitory, the level of phosphorylated histone H1, a substrate of CDK2, was determined as a readout of CDK2 activity (Lin Z P et al., 2014, Molecular Cancer Res, 12:381-393) to reconcile this discrepancy. DB4 shared a similarity with triapine as evidenced by down-regulation of phosphorylated histone H1 at 24 hr. Triapine reduced the level of phosphorylated histone H1 earlier starting at 3 hr, which correlated with the onset of Chk1 phosphorylation (FIG. 6D and FIG. 6E). However, DB4 did not induced Chk1 phosphorylation. The kinetics of CDK2 activity somewhat resembled to the level of DNA synthesis caused by DB4 and triapine. Thus, DB4 slowly decreased in CDK2 activity by down-regulating the level of cyclin A2, whereas triapine promptly reduced CDK2 activity by increasing Chk1-meditade inhibitory phosphorylation of CDK2.

To investigate the effects of DB4 on DSB end resection, the level of RPA32 phosphorylation, indicative of CDK2-dependent DSB end resection, was determined. Etoposide induced DSBs, leading to a marked increase in RPA32 phosphorylation in PEO4 cells (FIG. 6F). Triapine induced a minor RPA32 phosphorylation but blocked phosphorylation of RPA32, induced by etoposide. In contrast, DB4 had no effect on basal RPA32 phosphorylation and yet augmented etoposide-induced RPA32 phosphorylation.

Triapine induced ATR and Chk1 phosphorylation independently of etoposide treatment whereas DB4 only synergized with etoposide to induce Chk1 phosphorylation. DB4 appeared to weakly induce ATR phosphorylation. The synergy in Chk1 phosphorylation between DB4 and etoposide may result from enhanced RPA32 phosphorylation or DSB end resection. Collectively, these findings indicated that DB4 caused a gradual reduction in CDK2 activity allowing occurrence of DSB end resection and subsequent Chk1 activation. In contrast, triapine rapidly inactivated CDK2 primarily through Chk1 activation thereby blocking DSB end resection.

### DB4 Abrogated HR Repair in GFP Reporter and Nuclear Foci Assays

The ability of DB4 to inhibit endonuclease-induced DSB repair of reporter genes in SKOV3 cells was evaluated. SKOV3 cells are proficient in HR and classical NHEJ (cNHEJ). SKOV3-DR-GFP cells were transfected with an I-SceI expression plasmid to induce a DSB in the DR-GFP reporter integrated in the genome for assaying HR repair activity, as described previously (Lin Z P et al., 2014, Molecular Cancer Res, 12:381-393; Ratner E S et al., 2016, Br J Cancer, 114:777-786).

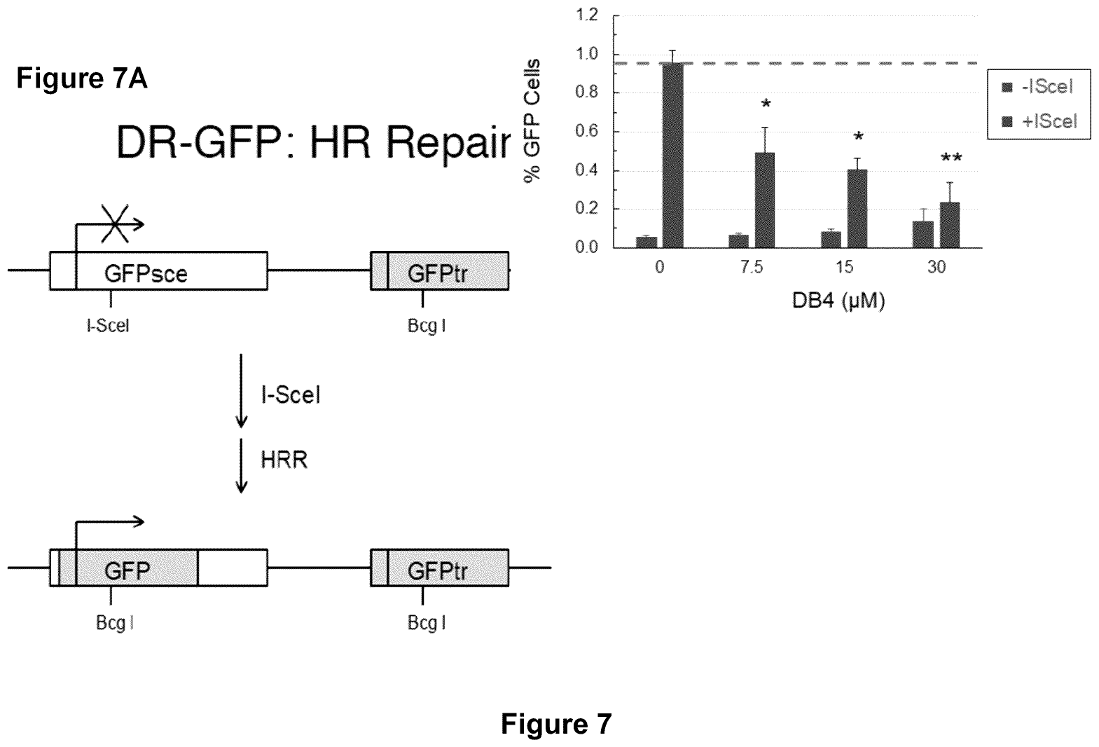

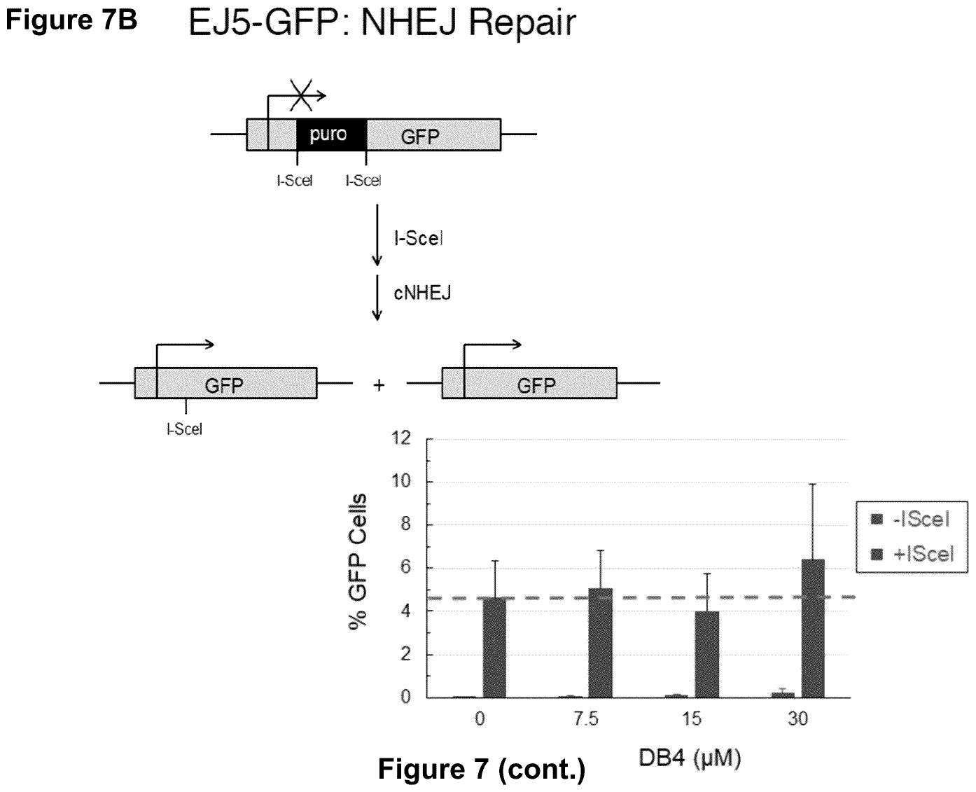

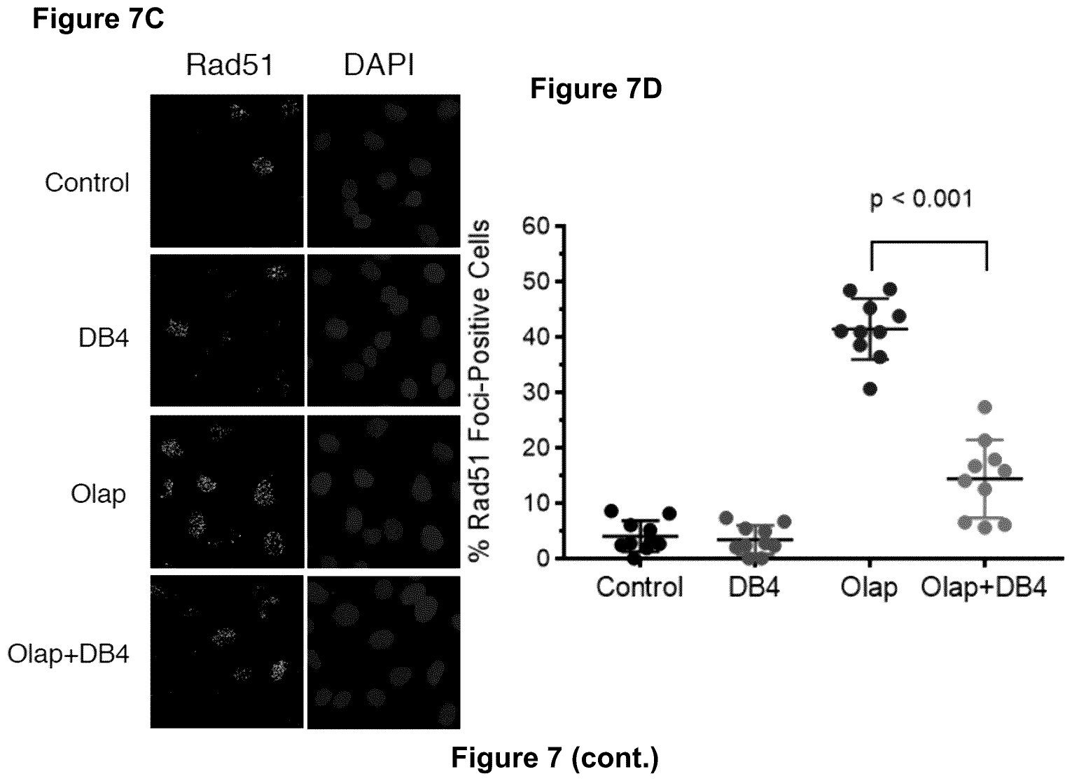

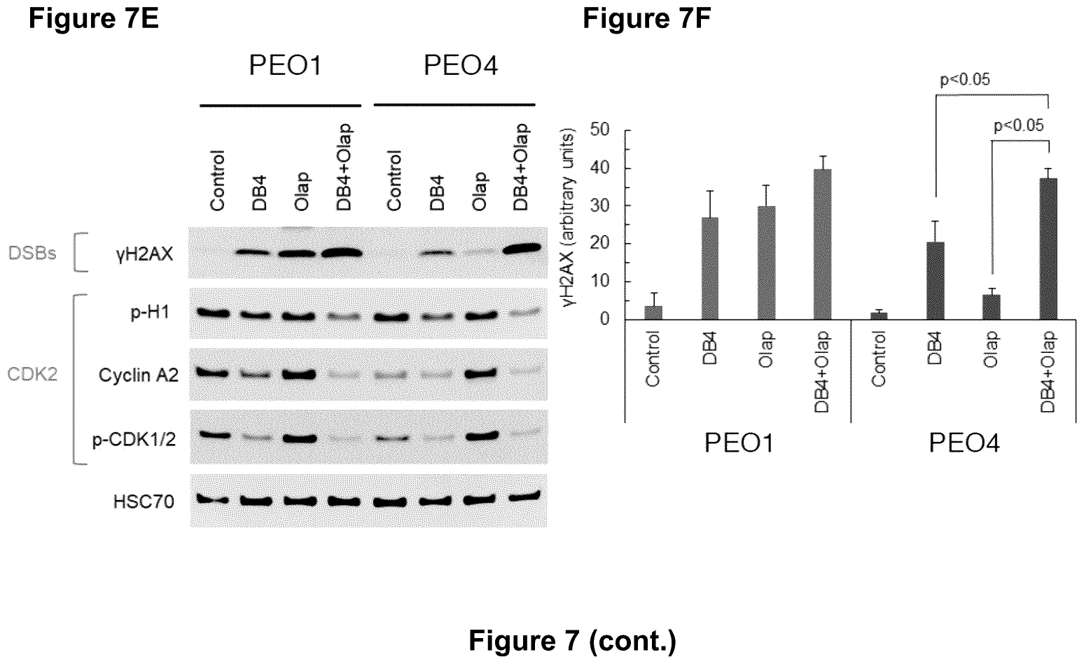

In addition, SKOV3-EJ5-GFP were transfected with an I-SceI expression plasmid to induce two DSBs in the EJ5-GFP reporter for assaying cNHEJ activity (Bennardo N et al., 2008, PLOS Genet, 4:e1000110). SKOV3-EJ5-GFP cells have been established by stable transfection of the EJ5-GFP construct. Treatment of these SKOV3 cells with DB4 led to significant inhibition of HR repair induced by an I-SceI-induced DSB in a dose dependent manner (FIG. 7A). In contrast, DB4 appeared to enhance the cNHEJ activity induced by I-SceI-mediated DSBs but the increase was not statistically significant (FIG. 7B). The IC₅₀ of DB4 for inhibiting HR repair was determined at 5.4 μM in SKOV3 cells. These results suggest that DB4 selectively inhibits HR-mediated DSB repair in SKOV3 cells.

To interrogate whether DB4 inhibited PARP inhibitor-induced DSB repair, nuclear Rad51 foci were determined in SKOV3 cells. Rad51 is a key component of the HR repair pathway. Treatment with DB4 had no effects on Rad51 foci, whereas treatment with olaparib induced a pronounced increase in Rad51 foci in SKOV3 cells. Importantly, DB4 significantly attenuated olaparib-induced Rad51 foci (FIG. 7C and FIG. 7D). These findings corroborated that DB4 inhibited HR repair in EOC cells.

Given that abrogation of HR repair hinders the recovery from DNA damage, additional studies investigated whether DB4 enhanced the accumulation of olaparib-induced DSBs as measured by the level of γH2AX in BRCA-wild type EOC cells. The combination of DB4 and olaparib led to a pronounced increase in the level of γH2AX significantly greater than DB4 or olaparib alone in PEO4 cells (FIG. 7E and FIG. 7F). In contrast, due to a lack of HR repair, the combination of DB4 and olaparib produced a greater but not significant increase in γH2AX than DB4 or olaparib alone in PEO1 cells. As judged by the level of phosphorylated histone H1, DB4 caused a decrease in cyclin A2 and CDK2 activity corresponding to an increase in γH2AX, confirming HR impairment in HR-proficient PEO4 cells.

### DB4 Enhanced DSBs and Sensitized BRCA-Wild Type EOC to PARP Inhibition

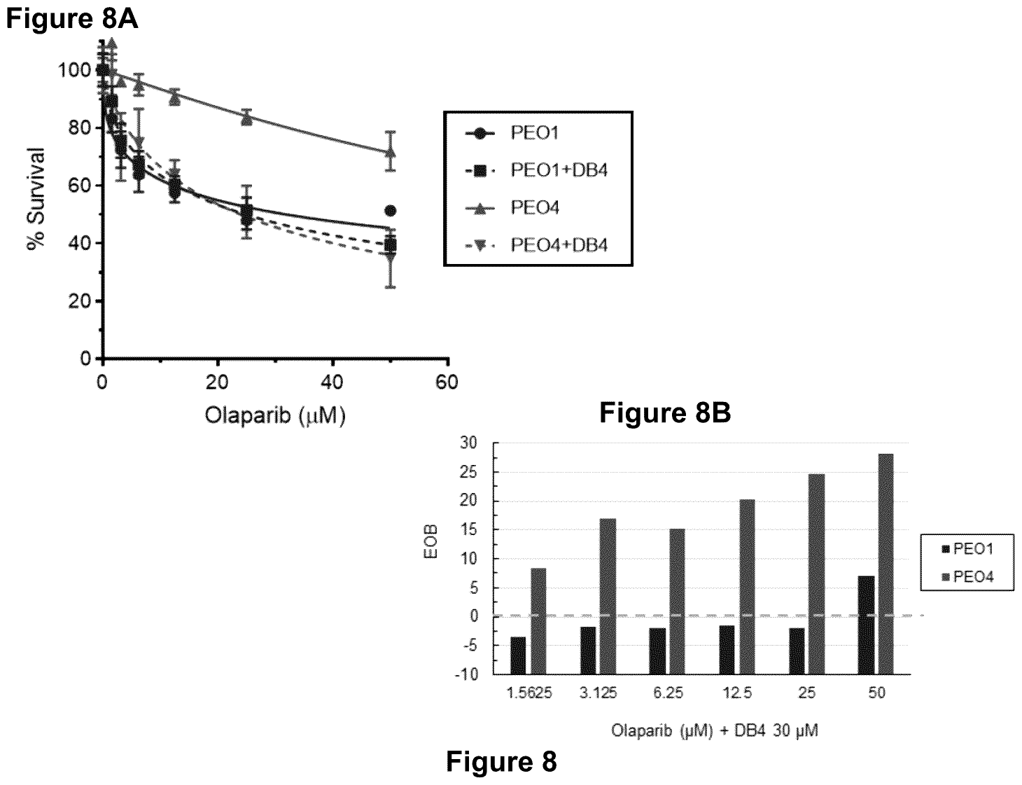

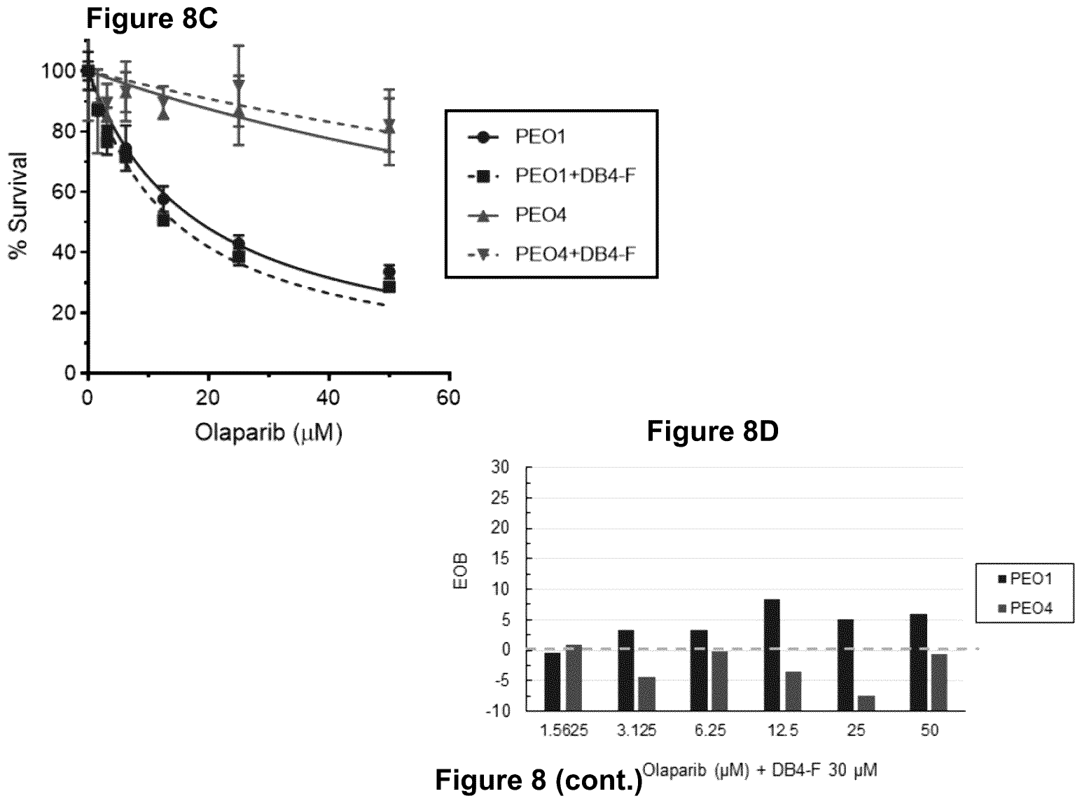

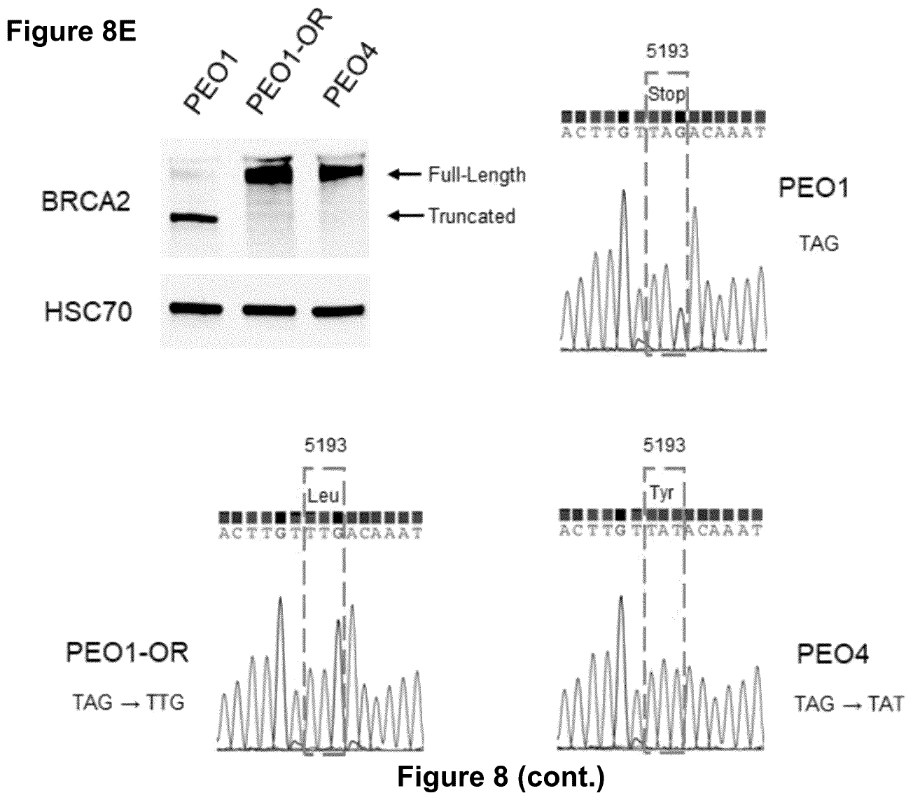

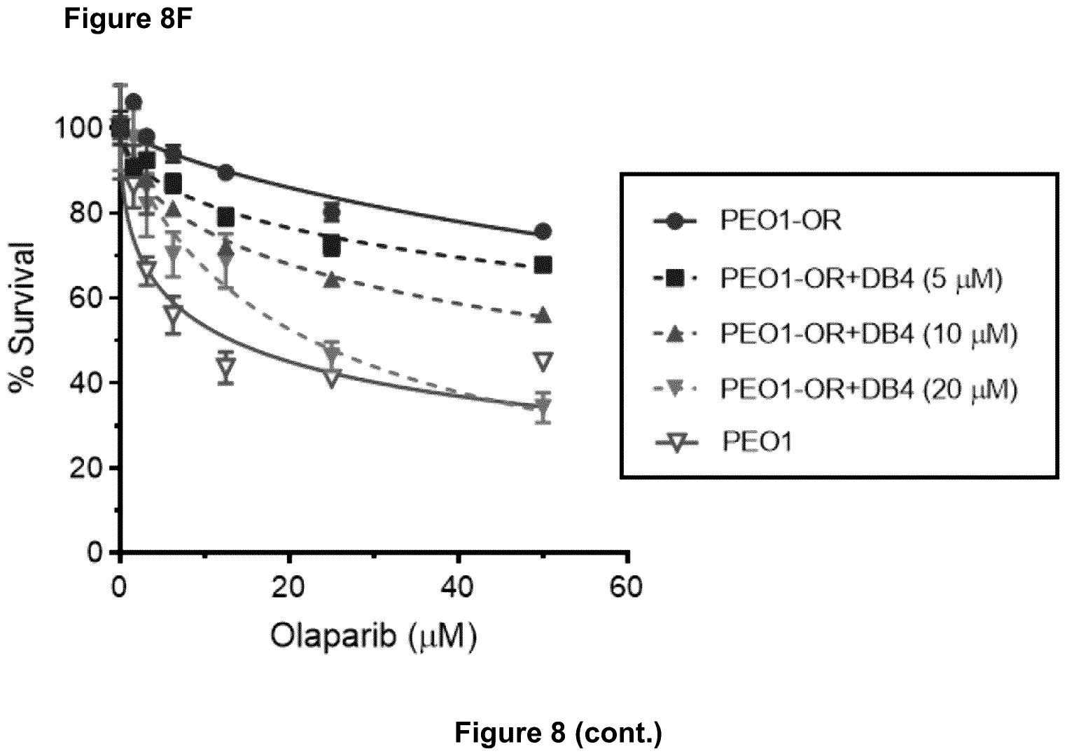

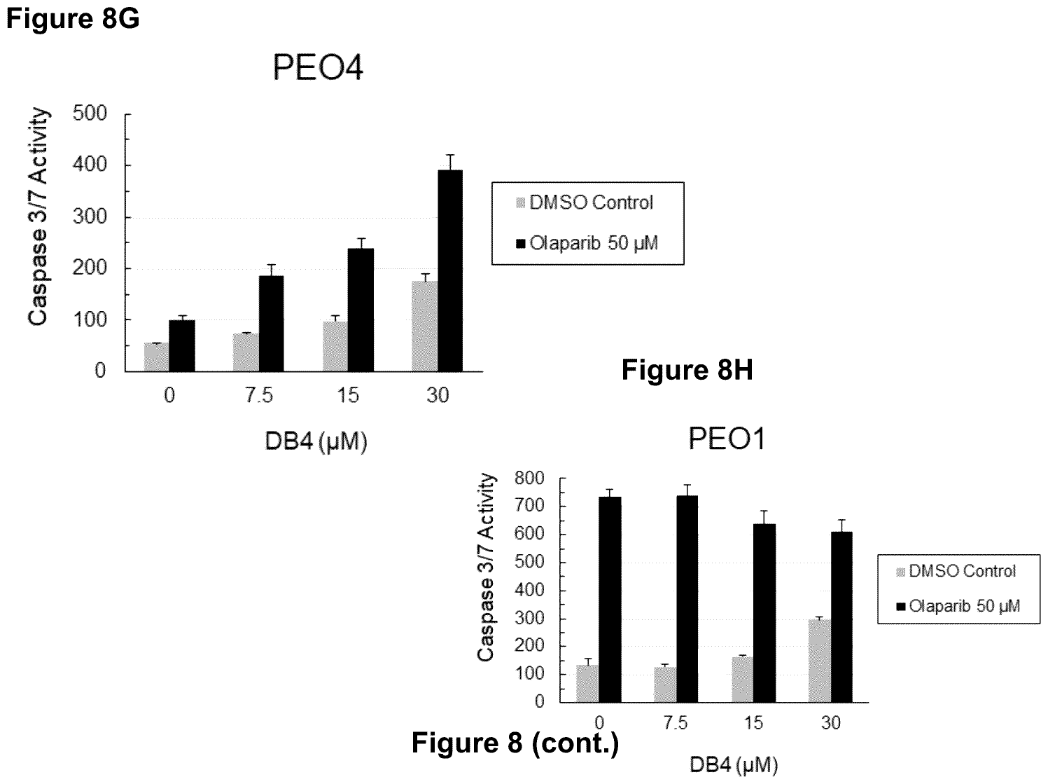

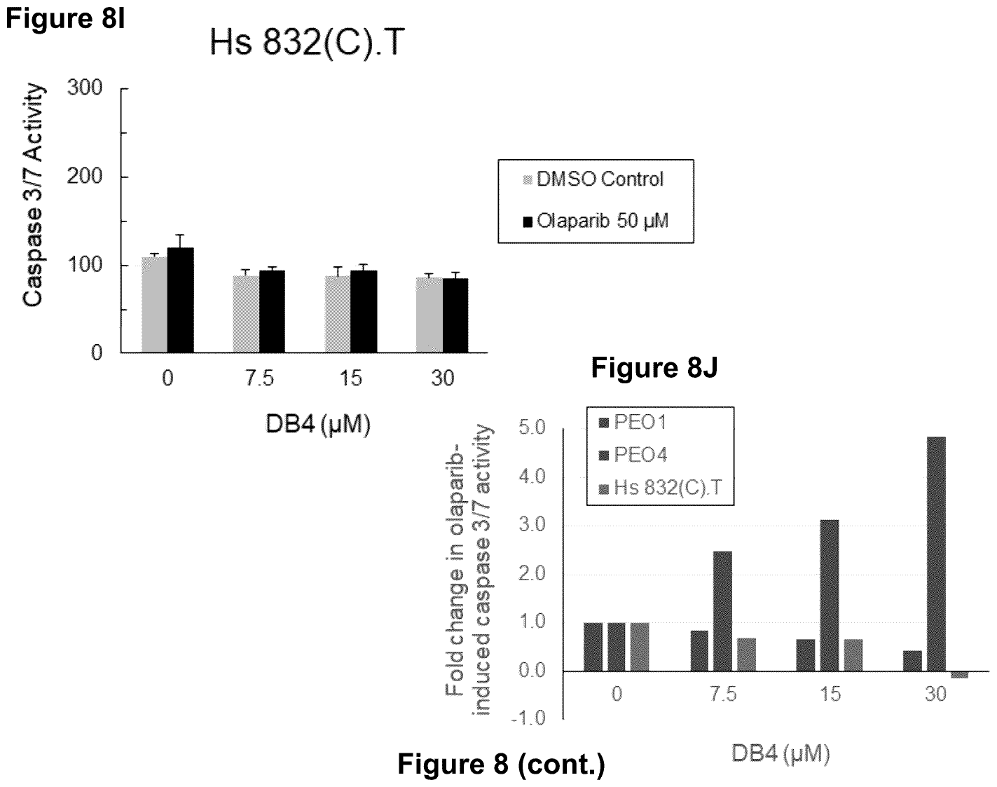

It was also examined whether DB4 sensitized BRCA-wild type EOC cells to a PARP inhibitor. BRCA2-mutated PEO1 and BRCA2-wild type PEO4 cells were treated with DB4 at 30 μM in combination with various concentrations of olaparib and cell survival was determined. It was found that DB4 effectively sensitized PEO4 cells to olaparib, while having minimal effects on PEO1 cells (FIG. 8A and FIG. 8B). Excess over Bliss (EOB) analysis for drug pair synergy also corroborated that the combination of DB4 and olaparib produced strong synergistic effects on PEO4 cells but had additive to slight antagonistic effects on PEO1 cells.

In addition, the effects of a DB4 analog, DB4-F, on the sensitivities of PEO1 and PEO4 cells to Olaparib were also tested. Because of a lack of inhibitory activity, DB4-F had no effects on the olaparib sensitivity of PEO1 cells and was unable to sensitize PEO4 cells to olaparib. (FIG. 8C and FIG. 8D).

Next, the concentrations of DB4 were varied to ascertain its effectiveness to cause sensitization to olaparib using a different BRCA-wild type EOC cell line. PEO1-OR cells, an olaparib-resistant PEO1 cell line derived from olaparib-sensitive PEO1 cells, was established and used because of a reverted BRCA2 mutation and restored wild type BRCA2 expression (FIG. 8E). PEO1-OR cells were sensitized by DB4 to various concentrations of olaparib in a dose-dependent manner (FIG. 8F). PEO1-OR cells were partially sensitized by DB4 at 5 μM, and near-fully sensitized to olaparib at 20 μM as compared with PEO4 cells at 30 μM. Collectively, these results confirmed that DB4 abrogated HR repair and therefore rendered BRCA-wild type EOC cells hypersensitive to a PARP inhibitor.

Furthermore, the effects of DB4 on olaparib-induced apoptosis at 24 hr were evaluated. Olaparib caused a minor increase in caspase 3/7 activity in PEO4 cells. DB4, starting at 7.5 μM, markedly augmented olaparib-induced caspase 3/7 activity in a dose dependent manner (FIG. 8G and FIG. 8J). In contrast, olaparib produced a pronounced increase in caspase 3/7 activity and DB4 failed to enhance olaparib-induced caspase 3/7 activity in PEO1 cells (FIG. 8H and FIG. 8J). Furthermore, the effects of DB4 on olaparib-induced apoptosis in non-malignant cells were investigated. Non-malignant cells arrest the cell cycle at the G1 phase and curtail HR repair in response to olaparib treatment. DB4 did not enhance the apoptotic effects of olaparib on the benign ovarian cyst fibroblast cells Hs 832(C).T (FIG. 8I and FIG. 8J).

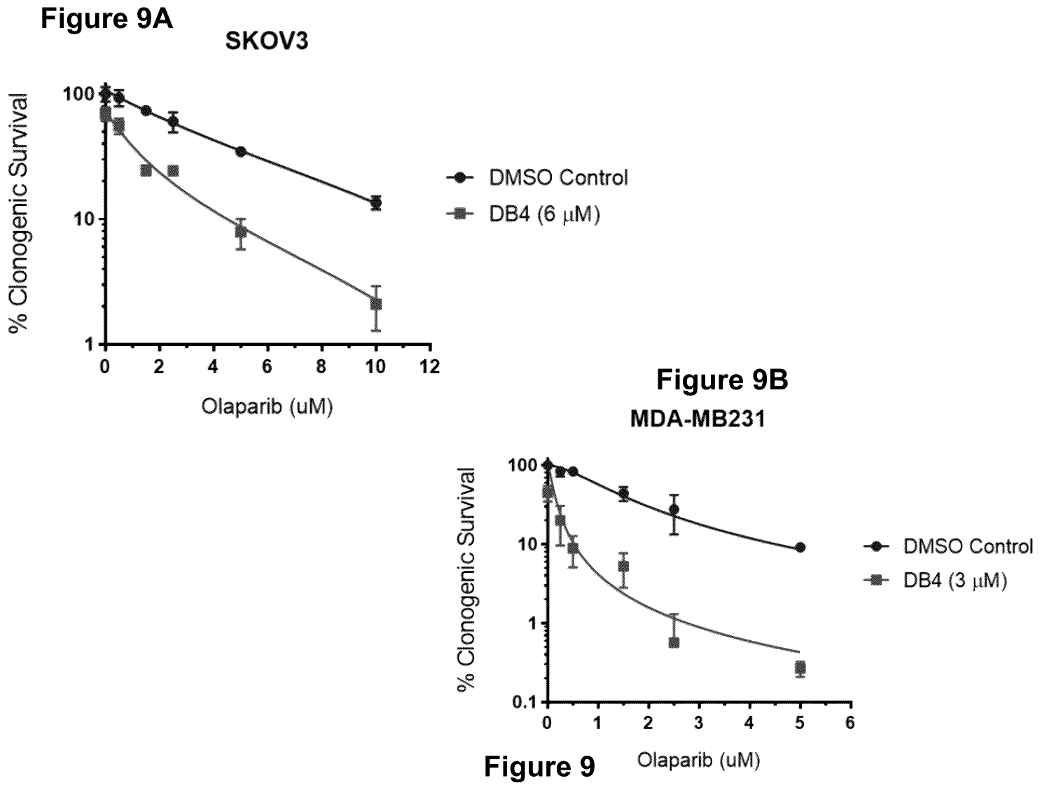

To further substantiate the effectiveness of DB4 and mitigate the issue of off-target effects with a higher concentration range of drugs used in the MTS cytotoxicity assay, clonogenic assays were carried out to determine the long-term survival of cells in response to treatments. The results showed that DB4 at 6 μM and 3 μM effectively reduced the clonogenic survival of BRCA-wild type SKOV3 EOC and MDA-MB231 breast cancer cells, respectively, in the presence of various lower concentrations of olaparib (FIG. 9).

### The Combination of DB4 and Olaparib Hindered the Peritoneal and Subcutaneous Progression of Olaparib-Resistant EOC Xenografts in Mice

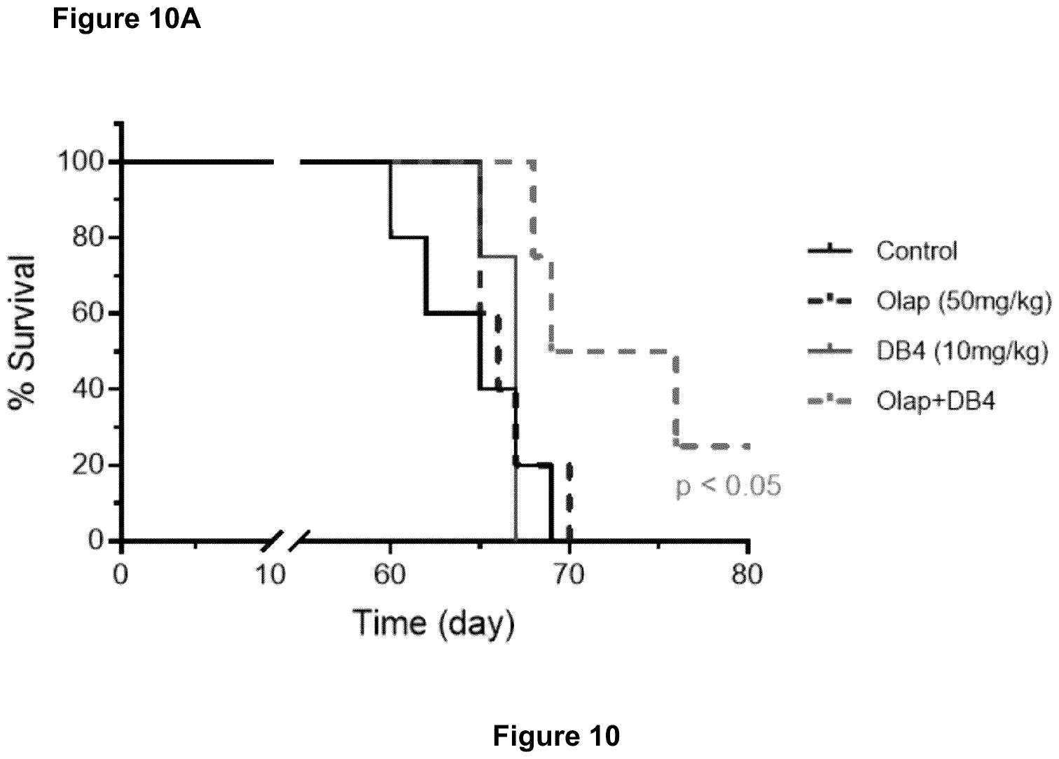

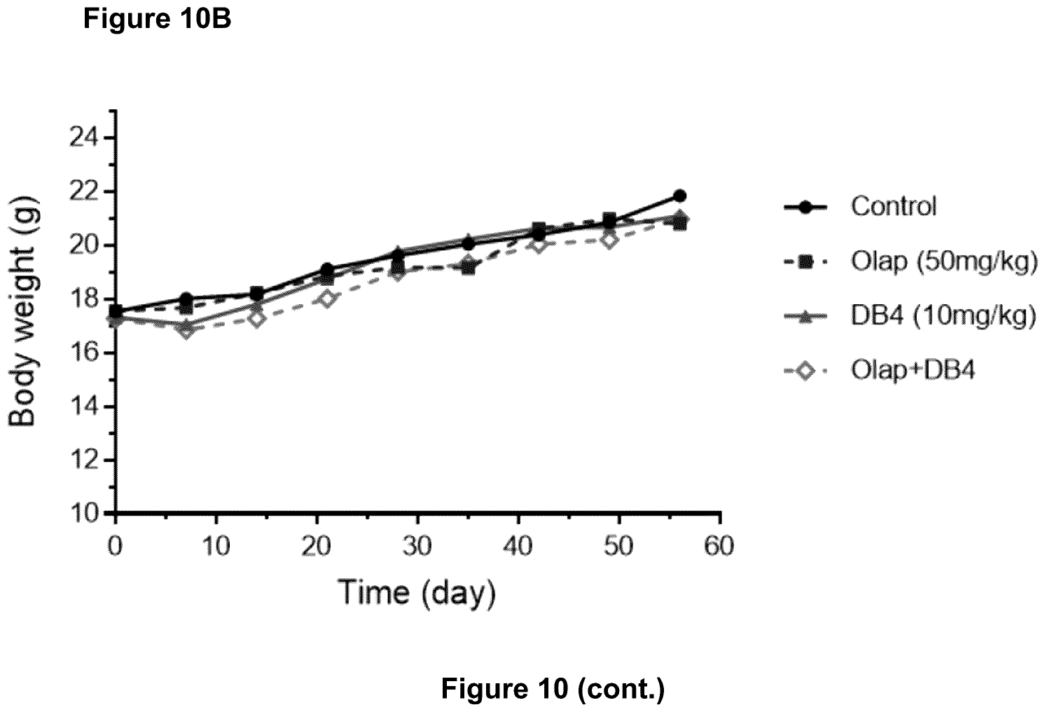

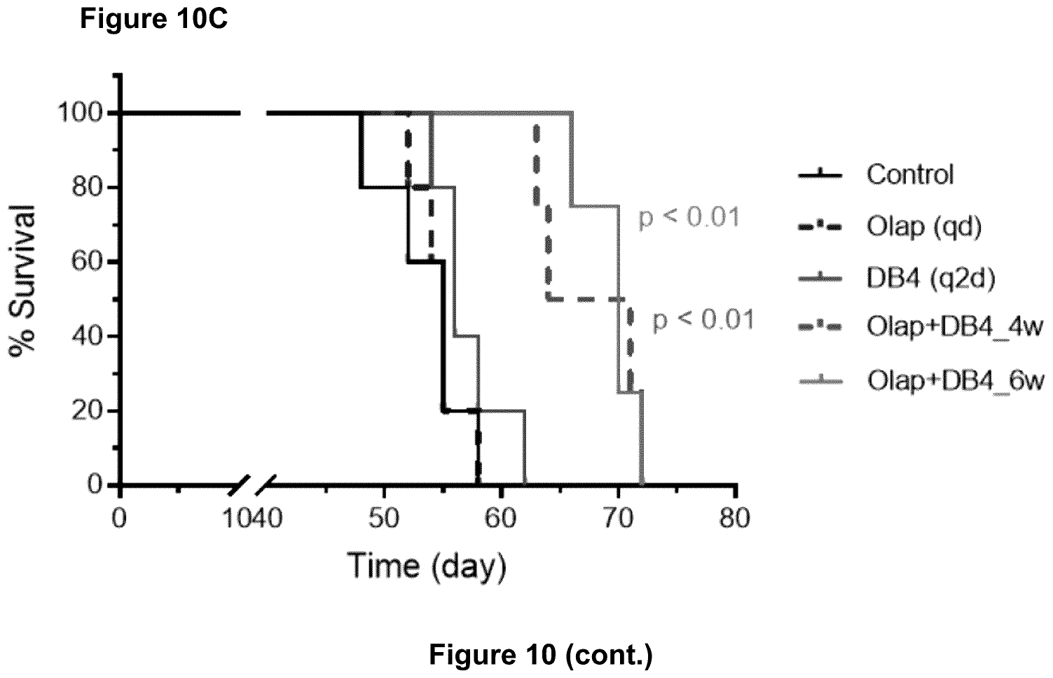

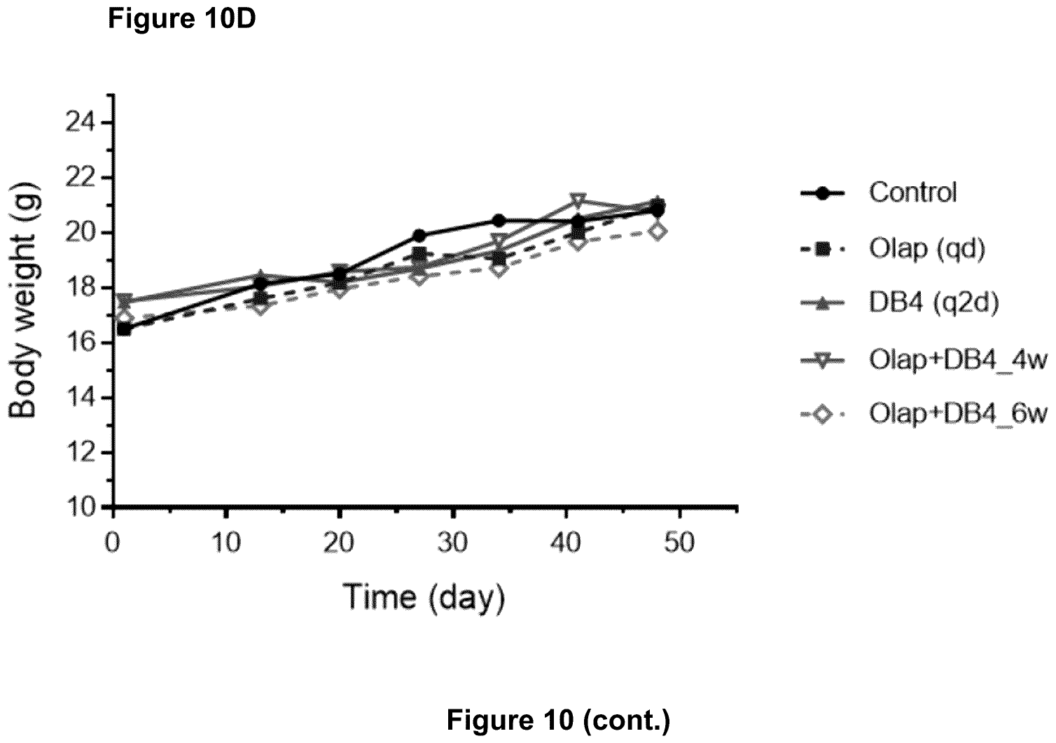

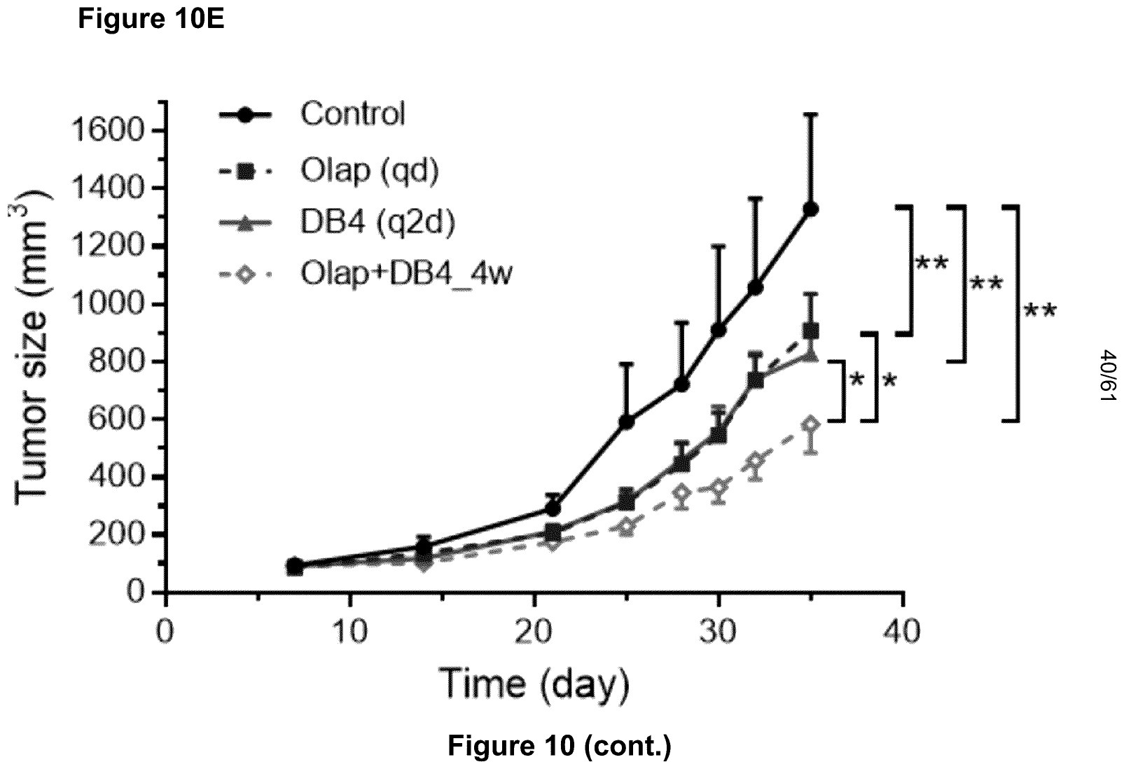

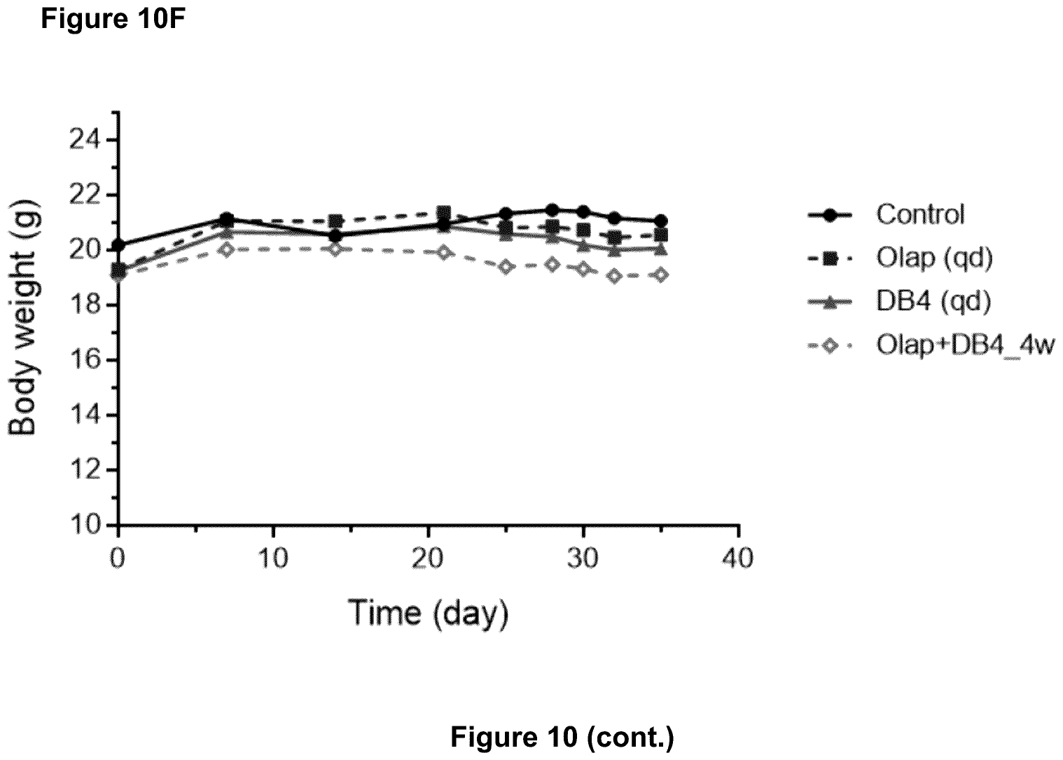

It was previously demonstrated that both PEO4ip and SKOV3 cells were BRCA-wild type and olaparib-resistant in cell-based assays and in vivo (Lin Z P et al., 2014, Molecular Cancer Res, 12:381-393; Ratner E S et al., 2016, Br J Cancer, 114:777-786; Lin Z P et al., 2018, PLOS One, 13:e0207399). First, the efficacy of the combination of DB4 and olaparib to treat PEO4ip tumor xenografts, as determined by the median survival time, was evaluated in a peritoneal mouse model (Lin Z P et al., 2018, PLOS One, 13:e0207399). Mice were implanted intraperitoneally (i.p.) with PEO4ip cells and then treated i.p. with olaparib, DB4, or both drugs in combination daily for 4 weeks. The survival endpoint (a 50% increase in abdominal circumference) for vehicle-treated control mice ranged from 55-65 days (8-9 weeks). Olaparib and DB4 alone had no effects on the survival time of mice (FIG. 10A and Table 2). In contrast, the combination of olaparib and DB4 moderately prolonged the survival time of mice (p<0.05).

TABLE 2
Median survival time of PEO4ip xenograft-bearing
SCID-Beige mice treated daily with olaparib,
DB4, and both drugs in combination for 6 weeks.

| Treatment | Median Survival Time (day) |
|---|---|
| Control | 65 |
| Olaparib (50 mg/kg, qd) | 66 |
| DB4 (10 mg/kg, qd) | 67 |
| Olaparib + DB4 | 73 |

Next, these experiments were repeated with modified treatment schedules of DB4 given every two day (q2d) for 4-week and 6-week treatment periods. The combination of DB4 and olaparib produced significant and further extension of survival time of mice (p<0.01) (FIG. 10C and Table 3). No apparent toxicity, as determined by changes in body weight of mice, was observed with either treatment schedules (FIG. 10B and FIG. 10D).

TABLE 3
Median survival time of PEO4ip xenograft-bearing SCID-
Beige mice treated with olaparib, DB4, and both drugs
in combination for varied dose intervals and durations.

| Treatment | Median Survival Time (day) |
|---|---|
| Control | 55 |
| Olaparib (50 mg/kg, qd) | 55 |
| DB4 (10 mg/kg, q2d) | 56 |
| Olaparib + DB4 (4 wks) | 68 |
| Olaparib + DB4 (6 wks) | 70 |

The efficacy of the combination of DB4 and olaparib to treat SKOV3 tumor xenografts, as determined by the tumor size, was ascertained in a subcutaneous (s.c.) mouse model (Ratner E S et al., 2016, Br J Cancer, 114:777-786; Lin Z P et al., 2018, PLOS One, 13:e0207399). Mice were implanted s.c. with SKOV3 cells and then treated i.p. with olaparib, DB4, or both drugs in combination for 4 weeks in the same manner as FIG. 10C. Olaparib or DB4 alone moderately but significantly inhibited subcutaneous growth of SKOV3 xenografts (p<0.01). Importantly, the combination of DB4 and olaparib resulted in concerted inhibition of tumor growth significantly greater than that caused by either olaparib or DB4 alone (p<0.05) (FIG. 10E and FIG. 10F).

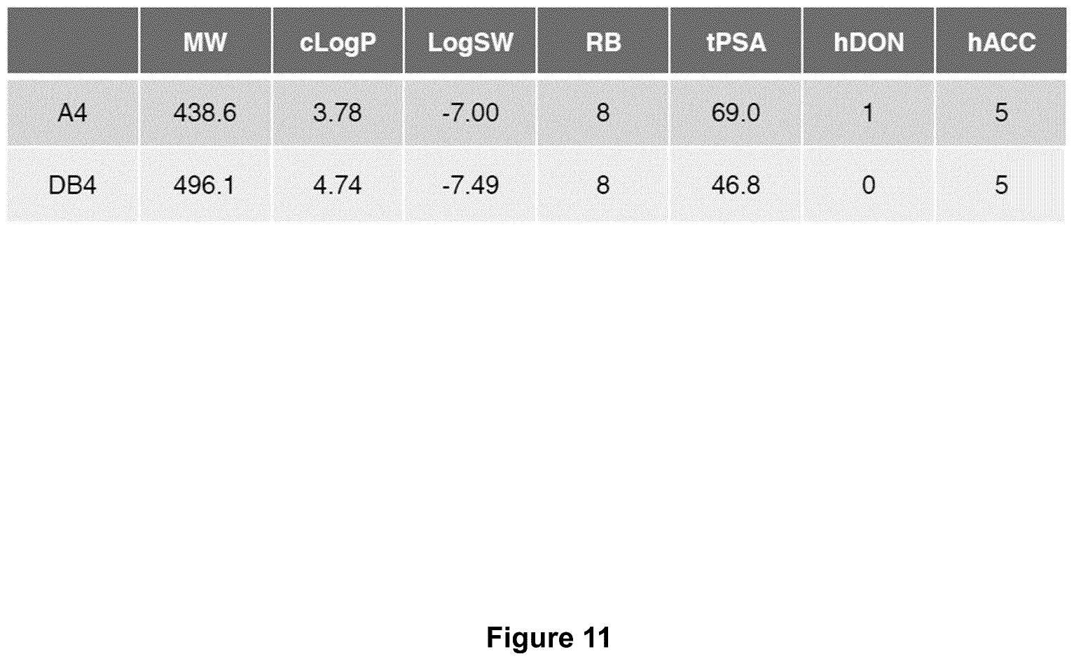

Using the in silico screening approach, DB4 was identified as a putative small molecule inhibitor of RNR that impairs HR repair for ovarian cancer therapy. This strategy for discovery of novel inhibitor compounds enabled enrichment of hit compounds for subsequent experimental validation, providing a much greater advantage over traditional high throughput screening. Because only 25 out of 200,000 compounds were examined experimentally, the true hit rate was 4%, which was substantially higher than the overall hit rate of 0.0005%. In addition to experimental validation of identifying true hits, analysis of physico-chemical properties of active compounds to enhance the likelihood of serving as a drug candidate were also conducted. According to Lipinski's rule of five (Lipinski C A et al., 2001, Adv Drug Deliv Rev, 46:3-26), both DB4 and A4 exhibited appropriate “druglikeness” as defined as a molecular mass less than 500 Daltons, an octanol-water partition coefficient not exceeding five, no more than five hydrogen bond donors, and no more than ten hydrogen bond acceptors (FIG. 11). Topological polar surface areas were optimal (<140), but aqueous solubilities were on the low side (<−4) (Sander T et al., 2015, J Chem Inf Model, 55:460-473).

In the present study, cytotoxicity and DNA synthesis inhibition assays were performed as primary follow-up experimental validation of in silico hits. The present studies took advantage of the fact that inhibition of RNR by small molecule compounds, such as hydroxyurea and triapine, caused a pronounced reduction in cell proliferation and DNA synthesis. RNR mediates the rate-limiting step in the production of dNTPs necessary for DNA synthesis (Lin Z P et al., 2004, J Biol Chem, 279:27030-27038; Lin Z P et al., 2011, Mol Pharmacol, 80:1000-1012). The dNTP measurement and dNs supplementing assays further implicated that DB4 inhibits RNR. However, although not bound by any particular theory, it is also possible that DB4 acts independently of RNR inhibition. To address this issue, additionally studies are exploring in vitro binding assays and in silico molecular dynamics simulation to facilitate the follow-up validation processes for hit discovery.

DB4 is one of the first-in-class of RNR inhibitors that does not involve quenching of tyrosyl-free radical for enzymatic inactivation. Notably, DB4 exhibited a distinct kinetics of DNA synthesis inhibition compared with triapine. It was previously shown that triapine rapidly depleted dNTPs, especially dATP and dGTP, and halted DNA synthesis within 1 hr (Lin Z P et al., 2004, J Biol Chem, 279:27030-27038; Lin Z P et al., 2011, Mol Pharmacol, 80:1000-1012). This was attributable to a prompt destruction of tyrosyl-free radical in the iron center of RNR (Sartorelli A C et al., 1976, Adv Enzyme Regul, 15:117-139). In contrast, DB4 caused a gradual reduction in DNA synthesis over the period of 24 hr.

Although not bound by any particular theory, it was hypothesized that triapine exhibited a greater potency and rate of inhibition by chemically inactivating RNR whereas DB4 reduced RNR activity by physically occupying the triapine-binding pocket of the R2 subunit. The function of the triapine-binding pocket remains largely unknown. Although not bound by any particular theory, it was hypothesized that that the pocket served as a channel involving the long-range proton-coupled electron transfer in RNR (Stubbe J et al., 2003, Chem Rev, 103:2167-2201). The free radical initiated from the iron center of the R2 subunit and propagated through the channel to the R1 subunit where the reduction of NDPs took place. Moreover, the triapine-binding pocket positions in a close proximity to the interface between R2 and R1 subunits (Stubbe J et al., 2003, Chem Rev, 103:2167-2201). Although not bound by any particular theory, it was hypothesized that the binding of DB4 to the pocket disrupted the interaction of R2 and R1 subunits and hindered the formation of enzymatically active RNR. Nevertheless, future investigation into the enzyme kinetics of RNR was merited to characterize the differing mode of action between DB4 and triapine.

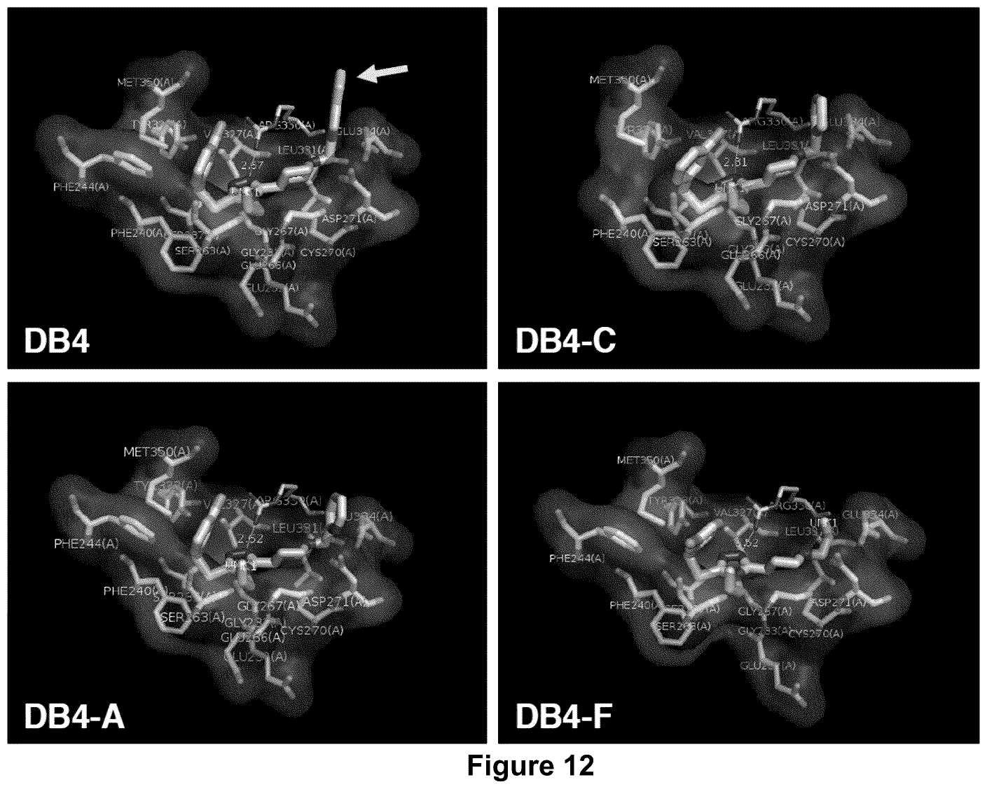

The preliminary SAR study indicated that the benzothiazole group of DB4 was critical because its absence or other functional group substitutions abolished the inhibitory activity in DNA synthesis (FIG. 3B and FIG. 4). Although DB4-A and DB4-C exhibited molecular interactions with the triapine binding pocket similar to DB4, the benzothiazole group of DB4 appeared to endow additional hydrophobic interactions (FIG. 3D). In addition, the fused benzene ring of the benzothiazole group, protruding above the triapine binding pocket, hinders the interaction between the R2 and R1 subunits. This structure was unique to DB4 compared with DB4-A and DB4-C(FIG. 12). Nevertheless, this supposition requires additional experimentation, including site-directed mutagenesis of key amino acid residues in the triapine-binding pocket of R2 subunit in vitro, evaluation of more DB4 analogs with substitutions of other functional groups, and X-ray crystallography of the R2-DB4 complex to substantiate the present model for the molecular docking of DB4.

Furthermore, the difference in the rate of RNR inhibition caused by DB4 and triapine provides an explanation for why DB4 did not abrogate DSB end resection in contrast to triapine. Because causing rapid depletion of dNTPs and stalling of DNA replication, triapine activates ATR and Chk1, thereby leading to prompt and complete inhibition of CDK2 and CtIP-mediated end resection of etoposide-induced DSBs (Lin Z P et al., 2014, Molecular Cancer Res, 12:381-393). In contrast, DB4 inhibited CDK2 activity but failed to block the end resection of DSBs caused by etoposide (FIG. 6F). Although not bound by any particular theory, it was hypothesized that DB4 caused dNTP depletion and CDK2 activity in a gradual fashion, thereby allowing DSB end resection to occur.

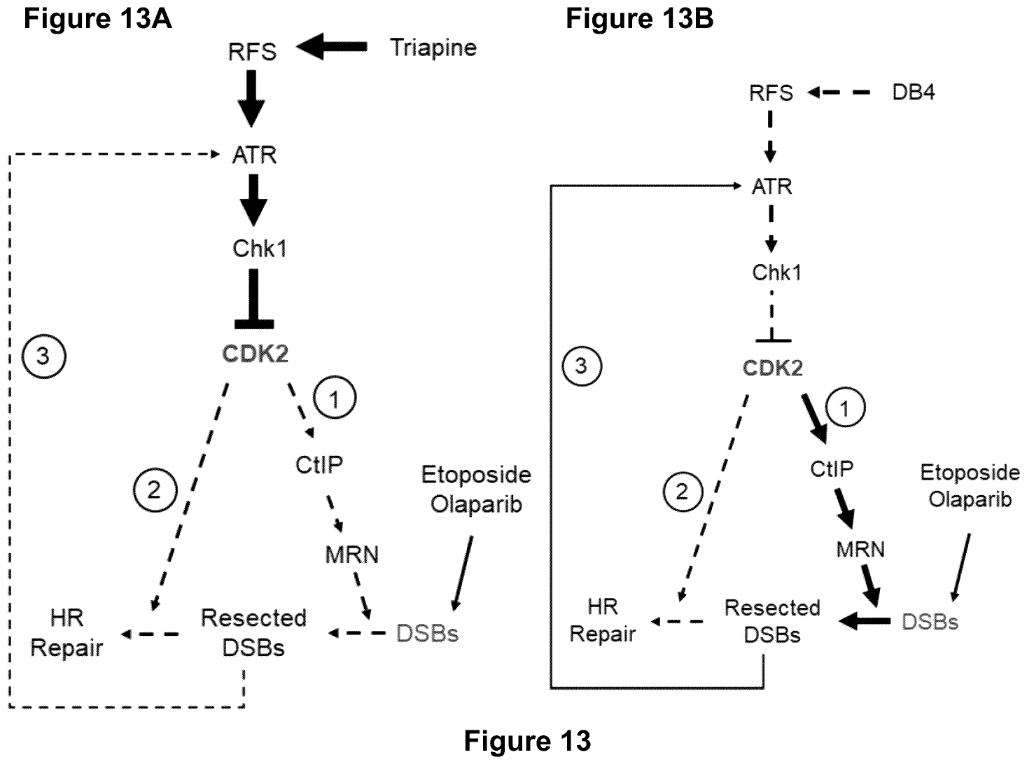

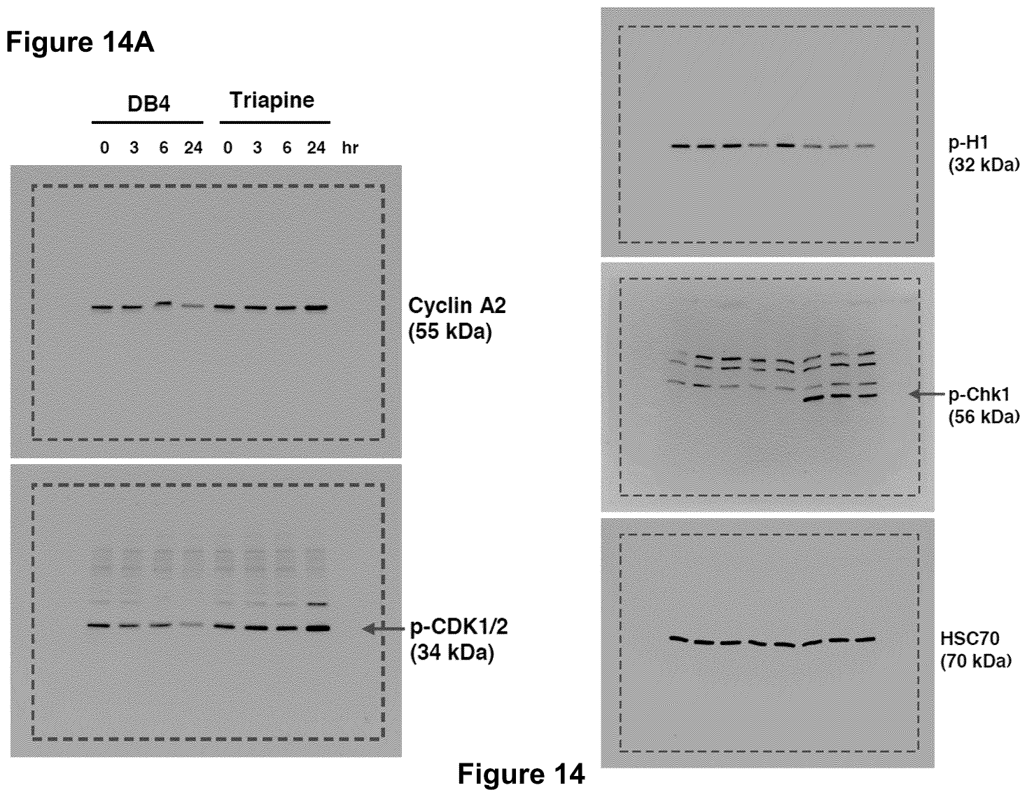

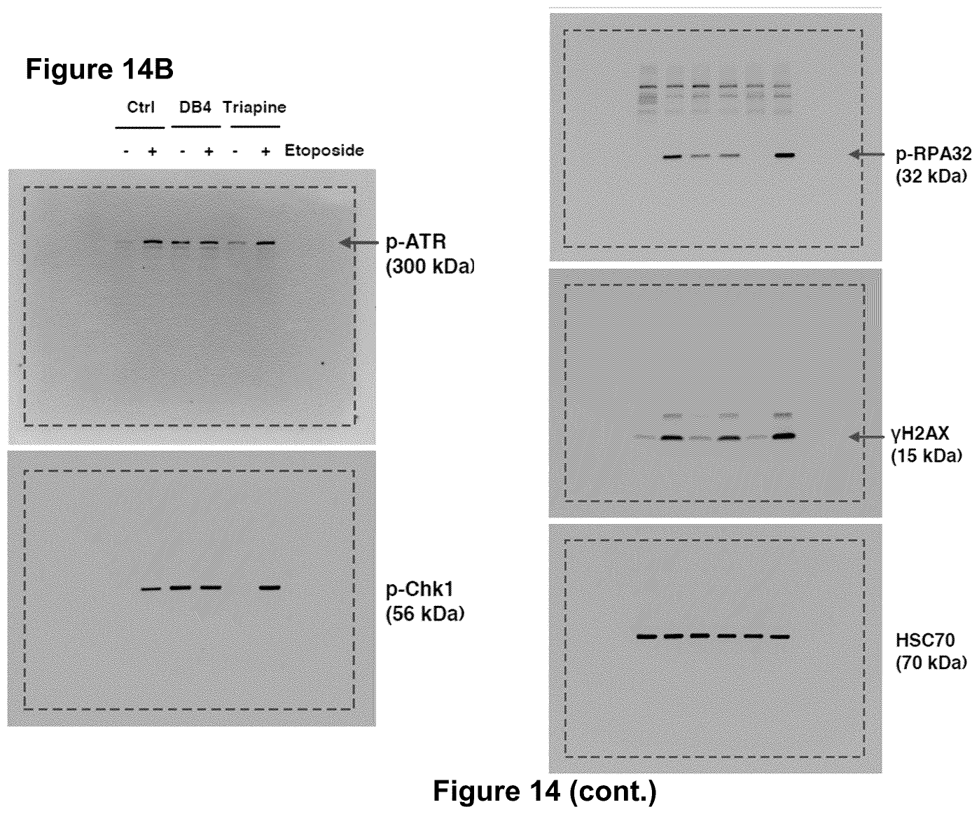

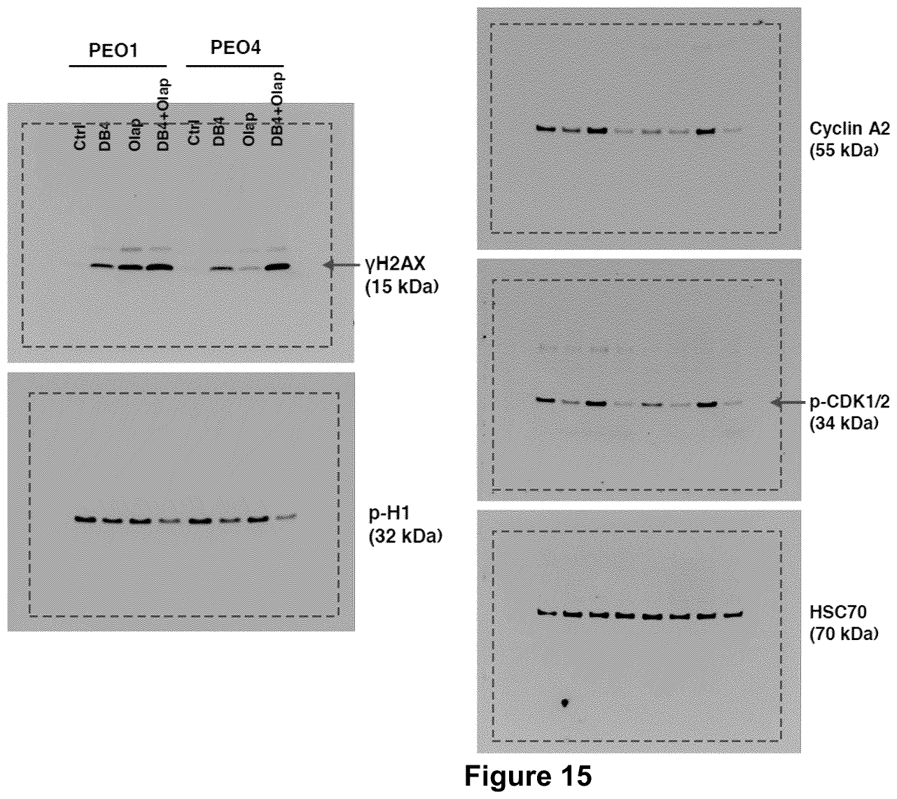

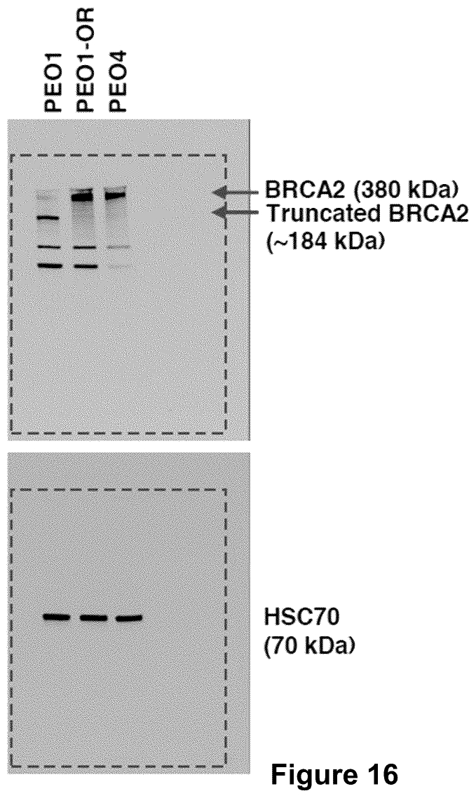

Differing from triapine, the activation of Chk1 by combined DB4 and etoposide treatment results from extensively resected DSBs. Thus, this indicated the models of triapine and DB4-mediated inhibition of CDK2 and HR repair as depicted in FIG. 13. Given that CDK2 activity was required for activation of many key components of DNA damage response and HR repair pathways, such as phosphorylation of Nbs1 on Ser432 (Wohlbold L et al., 2012, PLOS Genet, 8:e1002935) and RNF4 on Thr26 and T112 (Luo K et al., 2015, Nucleic Acids Res, 43:5465-5475), DB4 and triapine seemed to be equally effective to inhibit HR repair regardless of their abilities to block DSB end resection. The inhibitory effects of DB4 on CDK2 substrates during S and G2 phases of the cell cycle is being additionally investigated.

The present studies demonstrated that DB4 not only sensitized PEO4 cells but also PEO1-OR cells to the PARP inhibitor olaparib. PEO1 and PEO4 cells were derived from the same patient at first and second relapses, respectively, following platinum-based chemotherapy. Albeit being isogenic with altered BRCA2 and HR repair status, PEO4 cells may be clonally deviated from PEO1 cells because of tumor heterogeneity during disease progression in the patient. To mitigate this concern, BRCA2-wild type PEO1-OR cells were developed from BRCA2-mutated PEO1 cells in vitro to maintain the lineage of PEO1 background and minimize clonal variability and divergence. The present results substantiated that PEO4 and PEO1-OR cells are effectively and equally sensitized to olaparib by DB4. DB4 indeed abrogated restored HR repair proficiency in these PARP inhibitor-resistant cells. Importantly, DB4 did not sensitize non-malignant ovarian fibroblast cells to olaparib, indicating its specificity to EOC cells that heavily relied on HR repair for DNA damage and its minimal toxicity to normal tissue.

The in vivo studies showed that DB4 synergized with olaparib to hinder the progression of BRCA-wild type EOC xenografts and extended the survival time of tumor-bearing mice. It was noticeable that the combination regimen of DB4 given q2d was more efficacious than that of DB4 given qd to prolong the survival time of mice (FIG. 10B). Given the relatively slower kinetics of DB4, a reduced dosing frequency of DB4 is sufficient to achieve and maintain RNR/HR repair inhibition, with a less burden of RNR/HR inhibition-related toxicity to mice. Nevertheless, monitoring the body weight over the course of treatment strongly indicated this regimen was tolerable to mice even with severe immune deficiency. From clinical studies, triapine was known to have a short half-life in the plasma so a more frequent dosing schedule was considered to be preferable. Furthermore, DB4 is devoid of the adverse effects of triapine associated with iron-chelation including methemoglobinemia and dyspnea. Therefore, DB4 provides some advantages of pharmacokinetics over triapine for treating patients.

In conclusion, DB4 was identified as a potential new class of putative small molecule inhibitor of RNR for HR repair impairment. Since the effective concentration of DB4 for sensitizing BRCA wild-type cancer cells to olaparib was at single-digit μM level, the principal structure of DB4 offered a starting point for design and development of more potent derivatives with improved pharmacokinetic properties. Furthermore, a proof-of-concept approach of using DB4 to leverage PARP inhibitors for the treatment of resistant or refractory EOC was demonstrated. Further characterization of the mode of action and improvement of the efficacy of DB4 have meaningful impact on therapeutic innovation of EOC and provide a treatment option for patients who have PARP inhibitor-resistant EOC.

In summary, PARP inhibitors are promising targeted therapy for EOC with BRCA mutations or defective HR repair. However, reversion of BRCA mutation and restoration of HR repair in EOC lead to PARP inhibitor resistance and reduced clinical efficacy of PARP inhibitors. Previous studies have shown that triapine, a small molecule inhibitor of RNR, impaired HR repair and sensitized HR repair-proficient EOC to PARP inhibitors. However, adverse effects associated with the iron-chelating property of triapine are of concern for patients. To address these issues, in the present studies, in silico screening of small molecule libraries were performed to identify novel compounds that bind to the triapine-binding pocket on the R2 subunit of RNR and inhibit RNR in EOC cells.

Following experimental validation of selected top-ranking in silico hits for inhibition of dNTP and DNA synthesis, DB4, a putative RNR pocket-binding inhibitor markedly abrogated HR repair and sensitized BRCA-wild-type EOC cells to the PARP inhibitor olaparib, was identified. Furthermore, the present studies demonstrated that the combination of DB4 and olaparib deterred the progression of BRCA-wild type EOC xenografts and significantly prolonged the survival time of tumor-bearing mice. DB4 is a novel small molecule inhibitor that binds to a surface pocket on the R2 subunit of RNR. Its binding to the pocket resulted in RNR inhibition. DB4 was chemically inert. In contrast, triapine inhibited RNR by quenching the free radical of the R2 subunit of RNR. Triapine was chemically reactive and causing iron chelation. Thus, the mode of action of DB4 in RNR inhibition differed from and improved upon conventional RNR inhibitors (triapine, hydroxyurea, and many iron-chelators). This novel feature of DB4 circumvents potential side effects of triapine, including methemoglobinemia and dyspnea, in patients. Furthermore, DB4 is modified structurally to improve its binding affinity to RNR and PK/PD properties. Dependent largely on chemical reactivity for RNR inactivation, triapine has limited room for improvement on potency and binding affinity.

Given that PARP inhibitors demonstrated durable and safe clinical efficacy for patients with BRCA-mutated cancer, DB4 extended the indications of PARP inhibitors for the treatment of non-BRCA mutated cancers. Therefore, the present invention relates, in part, to DB4 that has a clear economic advantage over a long and costly process of new drug discovery for the treatment of non-BRCA mutated or PARP inhibitor-resistant cancers. As such, the present studies disclose the discovery of a putative small molecule inhibitor of RNR and HR repair for combination with PARP inhibitors to treat PARP inhibitor-resistant and HR repair-proficient EOC is reported.

The materials and methods employed in the present experiments are now described herein.

### Cell Lines and Chemicals

SKOV3 ovarian cancer cell line (ATCC; Manassas, VA) was grown in McCoys 5A medium supplemented with 10% FBS and penicillin-streptomycin antibiotics. PEO1 and PEO4 ovarian cancer, Hs 832(C).T ovarian fibroblast, and MDA-MB231 breast cancer cell lines were grown in DMEM medium supplemented with 10% FBS and penicillin-streptomycin antibiotics. PEO1 and PEO4 cell lines were confirmed by short-tandem repeat (STR) analysis (Promega-ATCC). PEO1-OR cells were established by chronically exposing PEO1 cells to 2.5 μM olaparib for 4 weeks and propagating surviving olaparib-resistant PEO1 cells. Ascites-derived PEO4ip cell line was also authenticated by STR analysis (Yale DNA Analysis Facility on Science Hill). Hs 832(C).T and MDA-MB231 cells were purchased from ATCC (Manassas, VA). Olaparib was purchased from Selleck (Houston, TX). Triapine (3-aminopyridine-2-carboxaldehyde-thiosemicarbazone) was synthesized as previously described (Ratner E S et al., 2016, Br J Cancer, 114:777-786). Screening compounds, DB4, and DB4 analogs were purchased from Chembridge (San Diego, CA).

### In Silico Screening, Docking, and Druglikeness Analysis

In silico screening, docking, scoring, and ranking of hit compounds was performed using the GOLD docking program (Cambridge Crystallographic Data Centre; Cambridge, UK). The database files of the compound library were obtained from Chembridge (San Diego, CA). The PDB file of the R2 subunit of RNR (2UW2) was loaded and prepared (protonation and tautomeric states). The triapine-binding pocket (Zhou B et al., 2013, Cancer Res, 73:6484-6493; Popovic-Bijelic A et al., 2011, J Inorg Biochem, 105:1422-1431) using the Gly233 residue as the central point of the pocket was defined as the binding site for local docking. Two subsets of the compound database as the SDF file format were used for docking into the pocket. Each compound was docked for 10 times. GOLDScore was chosen for scoring the ligand fitness by protein-ligand hydrogen bond energy, protein-ligand and ligand internal van der Waals energy, and ligand torsion strain. Following the run, the GOLDScore of each screened compound was obtained and exported as an Excel file and ranked. Two hundred top-ranked hit compounds were manually clustered based on common pharmacophores and structural similarities. For visualization of docking poses and rendering of protein structures, the PyMOL program (Schrödinger, Inc., New York, NY) was used. The schematic diagrams of protein-ligand interactions were analyzed and generated from the docking poses files using the LigPlot+ program (The European Bioinformatics Institute). Physical and chemical properties of hit compounds was determined from the Chembridge database files using the Data Warrior program (openmolecules.org/).

### Cytotoxicity/Viability Assay

The assay was performed as described previously (Lin Z P et al., 2004, J Biol Chem, 279:27030-27038; Lin Z P et al., 2011, Mol Pharmacol, 80:1000-1012). PEO1, PEO1-OR, and PEO4 cells were plated into 96 well plates for 24 hr. Cells were then treated with drugs in triplicate or quadruplicated wells for 72 hr. Thereafter, 20 μL of CellTiter 96 AQueous MTS Reagent (Promega; Madison, WI) was added to wells for additional 2 hr incubation and the plates were immediately read by a colorimetric plate reader at wavelength of 490 nm. The absorbance was calculated to determine the percentage of cell survival relative to vehicle-treated controls.

### Clonogenic Assay

The assay was conducted as described previously (Lin Z P et al., 2014, Molecular Cancer Res, 12:381-393). Cells were plated at various densities in 6-well plates in triplicate. After 24 hr of incubation, cells were treated continuously with various concentrations of olaparib, DB4, or both drugs in combination. After 12-14 days, colonies were fixed/stained with crystal violet solution and counted to determine the percentage of survival using a GelDoc imaging system with QuantityOne software (Bio-Rad, Hercules, CA).

### DNA Synthesis Assay

During the final hour of drug treatment, cells were pulse-labeled with 10 μM EdU (5-ethynyl-2′-deoxyuridine). Cells were then collected, fixed, permeabilized for detection of S phase population using Click-iT EdU Alexa Fluor 488 Flow Cytometry Assay Kit (Thermo Fisher Scientific, Waltham, MA), followed by counterstaining of DNA with 7-aminoactinomycin D (7-AAD) (BD Biosciences, Franklin Lakes, NJ). Bivariate analysis of EdU incorporation and DNA content was performed by flow cytometry using LSRII flow cytometer (BD Biosciences) and FlowJo software (FlowJo LLC, Ashland, OR). The S phase cell population was gated to determine the percentage of cells undergoing DNA synthesis.

dNTP Measurement

The cellular levels of dNTP determination were carried out using the HIV-1 RT-based dNTP assay as described previously (Diamond T L et al., 2004, J Biol Chem, 279:51545-51553). Approximate 2-3×10⁶ cells were treated with drugs for 24 hr, trypsinized, counted, and washed with PBS twice. Cell pellets were extracted with 65% methanol at 95° C. for 3 min. The level of each of dNTP was determined and expressed as pmole/10⁶ cells.

### Liquid Chromatography-Multiple Reaction Monitoring-Mass Spectrometry (LC-MRM-MS) Analysis

DB4 was dissolved in DMEM medium at 50 μM and incubated for 0 hr, 24 hr, 48 hr, and 72 hr at 37° C. and flash frozen after incubation. A 2 μL aliquot was injected and separated using an Agilent 1290 Infinity UPLC system with a reversed-phase column (Zorbax Eclipse Plus Rapid Resolution HD, 2.1×50 mm, 1.8 μm C18, Agilent) at room temperature. Mobile Phase A was 0.1% formic acid in water, mobile phase B was 0.1% formic acid in acetonitrile (LC-MS Optima Fisher). Gradient was 100% A for 1 min offline to desalt the sample, at 0.4 mL/min flow followed by a linear gradient to 98% B over 5 min at 0.4 mL/min. The qToF (Agilent 6550) was operated in positive scanning mode (50-1000 m/z) and the following source parameters: VCap:3500 V, nozzle voltage: 2000 V, gas temp: 225° C.; drying gas 13 L/min; nebulizer: 20 psig; sheath gas temp 225° C.; sheath gas flow 12 L/min. Online mass calibration was performed using a second ionization source and a constant flow (5 μL/min) of reference solution (121.0509 and 922.0098 m/z). Compounds were identified based on the retention time of chemical standards and their accurate mass (tolerance 20 ppm).

### Western Blot Analysis

The methodology was described previously (Lin Z P et al., 2014, Molecular Cancer Res, 12:381-393; Ratner E S et al., 2016, Br J Cancer, 114:777-786). The anti-HSC70 antibody was purchased from Santa Cruz (Santa Cruz, CA). Anti-cyclin A2, anti-phospho-ATR (Thr-1981), anti-phospho-Chk1 (Ser-345), anti-phospho-CDK1/2 (Tyr-15), and anti-Y-H2AX (Ser-139) antibodies were from Cell Signaling (Danvers, MA). Anti-phospho-RPA32 (S4/S8) and anti-BRCA2 antibodies were from Bethyl Laboratories (Montgomery, TX). Anti-phospho-histone H1 antibody was from Millipore-Sigma (Burlington, MA). To avoid the cross-reaction of secondary antibodies with previously probed primary antibodies, blots were stripped and re-probed with next primary antibodies raised in a different species in an alternate fashion. All images were acquired and processed using the G: BOX gel documentation system and the GeneSnap software (Syngene, Frederick, MD). Brightness and contrast of images were applied equally across the entire gel/blot image and to controls. Images of protein bands were cropped and only brightness-adjusted using the PowerPoint software (Microsoft, Redmond, WA). Quantification of the protein band intensity was performed using the ImageJ software (NIH, Bethesda, MD).

### Caspase 3/7 Assay

The assay was performed as described previously (Lin Z P et al., 2018, PLOS One, 13:e0207399). At the end of 24 hr drug treatment, cells were lysed with the lysis buffer (PBS, 1% NP40, 0.1% SDS). 10 μL of lysate was incubated with Caspase-Glo 3/7 Assay reagent (Promega) at room temperature for 1 h and luminescence was subsequently measured with a luminometer (Turner Designs/Promega). Total protein concentration of cell lysates was determined as described above. Caspase 3/7 activity was expressed as luminescence units (RLU) per μg/μL protein for each sample.

### HR and NHEJ Reporter Assays

SKOV3-DR-GFP cells were established as described previously (Lin Z P et al., 2014, Molecular Cancer Res, 12:381-393). SKOV3-EJ5-GFP cells were established by stable transfection of SKOV3 cells with pimEJ5-GFP plasmid (Bennardo N et al., 2008, PLOS Genet, 4:e1000110) (Addgene, Watertown, MA). Cells were transiently transfected with the empty vector pRc/CMV/Neo (Thermo Fisher Scientific) or the I-SceI endonuclease expression vector pCBASceI (Addgene) (Richardson C et al., 1998, Genes Dev, 12:3831-3842) using Lipofactamine 2000 (Thermo Fisher Scientific) according to the manufacturer's protocol. 2 hr after transfection, cells were treated with various concentrations of DB4 for 48 hr. Cells were then trypsinized, stained with 7-AAD, and analyzed for the percentage of GFP-positive and 7-AAD-positive cells by flow cytometry using an LSR II flow cytometer (BD Biosciences) and FlowJo software (Tree Star; Ashland, OR). The ratio of %7-AAD-positive cells was used to normalize the percentage of GFP-positive cells.

### Immunofluorescent Staining and Confocal Microscopy

SKOV3 cells were grown for 24 hr prior to drug treatment. Cells were then fixed with 2% formaldehyde and permeabilized in cooled 100% methanol. The slides were blocked with 3% bovine serum albumin and stained with the rabbit primary anti-Rad51 antibody (Thermo Fisher Scientific) followed by incubation with the AlexaFluor 488-conjugated anti-rabbit secondary antibody (Thermo Fisher Scientific). Slides were then mounted with coverslips using the Prolong Gold Anti-Fade reagent (Thermo Fisher Scientific) containing DAPI for nuclei counterstaining. Immunofluorescence of Rad51 proteins and nuclei were acquired with a Leica SP5 confocal microscope (Wetzlar, Germany). Nuclei containing equal or more than 10 Rad51 foci were counted to determine % Rad51-positive cells.

### Excess Over Bliss (EOB)

Effects of two drugs in combination on cell survival were determined by EOB based on the principle of Bliss independence (Bliss C I et al., 1939, Ann. Appl. Biol., 26:585-615). The value of EOB was calculated by the formula: EOB=(Ev−Cv)×100, where Ev is the experimental value and Cv is the calculated value. Cv=Ea+Eb−Ea×Eb, where Ea is the fraction affected by drug a and Eb is the fraction affected by drug b. EOB>0 indicates synergy, EOB=0 indicates additivity, and EOB<0 indicates antagonism.

### Statistical Analysis

Statistical analysis for the effects of treatments in cell-based assays was performed by ordinary one-way ANOVA with Holm-Sidak's multiple comparisons test, using the Prism software (GraphPad, La Jolla, CA). All tests were two-tailed with an a level at 0.05.

### Tumor Xenografts, Drug Treatments, and Tumor Growth Evaluation

The Yale University Institutional Animal Care and Use Committee approved the protocol (IACUC #2018-20038) for the in vivo animal studies in compliance with the US Public Health Policy on Humane Care and Use of Laboratory Animals. Yale University is registered as a research facility with the United States Department of Agriculture, License and Registration number 16-R-0001 The School of Medicine is fully accredited by the American Association for Accreditation of Laboratory Animal Care (AAALAC). An Animal Welfare Assurance (D16-00146) is on file with OLAW-NIH; Approval Period: May 1, 2019-May 31, 2023.

Five to six weeks old female SCID-Beige mice were purchased from Envigo (Indianapolis, IN). PEO4ip cells (Lin Z P et al., 2018, PLOS One, 13:e0207399) suspended in 100 μL serum-free medium were implanted i.p. (1×10⁷ cells per mouse). Six to eight weeks old female NCG (NOD-Prkdcem26Cd52Il2rgem26Cd22/NjuCrl) mice were purchased from Charles River (Wilmington, MA). SKOV3 cells suspended in 100 μL serum-free medium were mixed with 50 μL Matrigel (BD Biosciences) and implanted s.c. in the right dorsal medial area (1×10⁷ cells per mouse). PEO4ip xenografts exhibited peritoneal progression as evidenced by ascitic development and abdominal distension. The abdominal circumference of PEO4ip-bearing mice reached a 50% increase about 50-70 days. SCID-Beige mice bearing PEO4ip xenografts exhibited 95% tumor take rate. NCG mice bearing SKOV3 xenografts displayed 100% tumor take rate.

Mice were randomly assigned to treatment groups (n=4-5) and drug treatment was initiated 3-7 days after tumor implantation. The control group of mice received i.p. treatment with vehicle (2% DMSO) and treatment groups of mice received i.p. treatment for a period of 4 or 6 weeks as follows: Olaparib (50 mg/kg) once daily for 5 consecutive days per week; DB4 (10 mg/kg) once daily for 5 consecutive days (M, T, W, R, F) per week or once every 2 days (M, W, F) per week; The combined treatment with olaparib and DB4 at the dose and schedule described above.

The progression of PEO4ip tumor xenografts in mice was evaluated by measuring the size of the circumference at the lower one-third of the abdominal area with the mouse head positioned upward. A 50% increase in the abdominal circumference was defined as the endpoint of survival as described previously (Lin Z P et al., 2018, PLOS One, 13:e0207399). In contrast, the cohort mice without tumor implantation exhibited 15-17% increase in abdominal circumference at the time when vehicle treated PEO4ip-bearing mice reached a 50% increase in the abdominal circumference. Kaplan-Meier survival curve, median survival time, and statistical analysis was performed using the Prism software (GraphPad). Comparisons were made by the Mantel-Cox test comparing the control with each treatment group. The growth of SKOV3 tumor xenografts was evaluated by the size of s.c. tumor using a digital caliper. Tumor volumes were calculated using the formula: length×width2/2. Statistical difference in the tumor size was determined by the Wilcoxon matched-pairs signed test comparing the control with each treatment group and comparing between treatment groups. Body weights of mice were measured on every treatment day before administration and on the same schedule as tumor measurements during the treatment period.

### Example 2: Subsequent Studies of in Silico Hit and Experimentally Active RNR Inhibitors on In Vitro and In Vivo Dosage Comparison, Pharmacophore Modeling, and Combination with Immune Checkpoint Blockade to Treat Ovarian Cancer

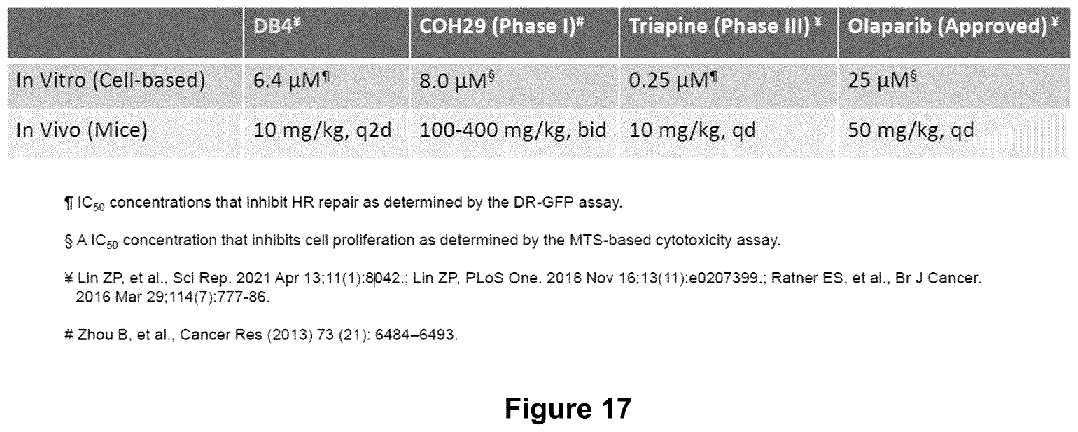

The comparative evaluation of in vitro and in vivo dosages of DB4 and other clinically used and FDA-approved RNR and PARP inhibitors was subsequently performed. As shown in FIG. 17, DB4, COH29, and triapine were used as exemplary RNR inhibitors and olaparib was used as an exemplary PARP inhibitor. DB4 was demonstrated to exhibit in vitro and in vivo potencies comparable to other clinically used and FDA-approved RNR and PARP inhibitors.

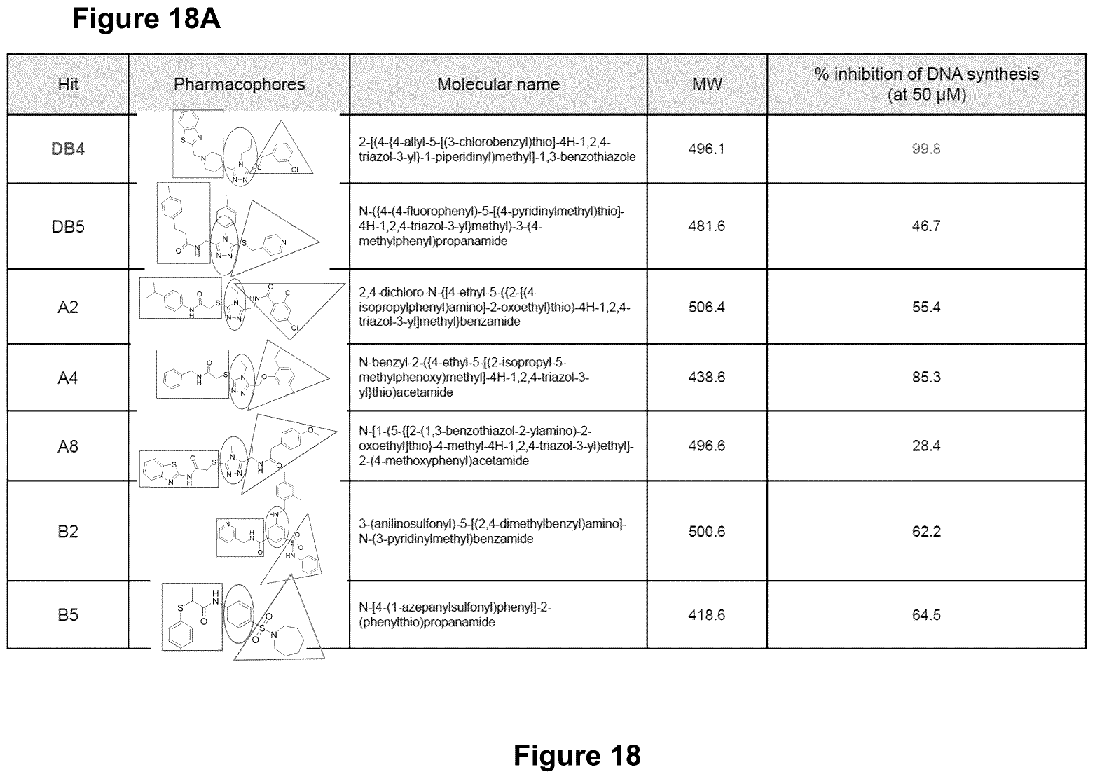

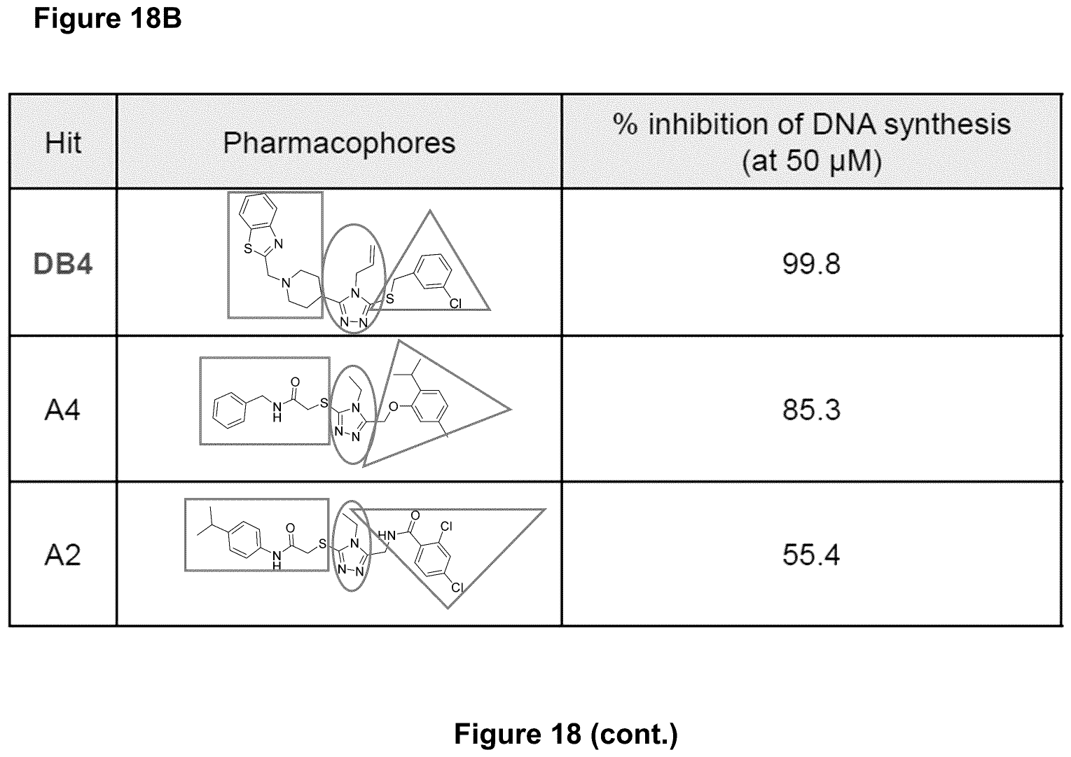

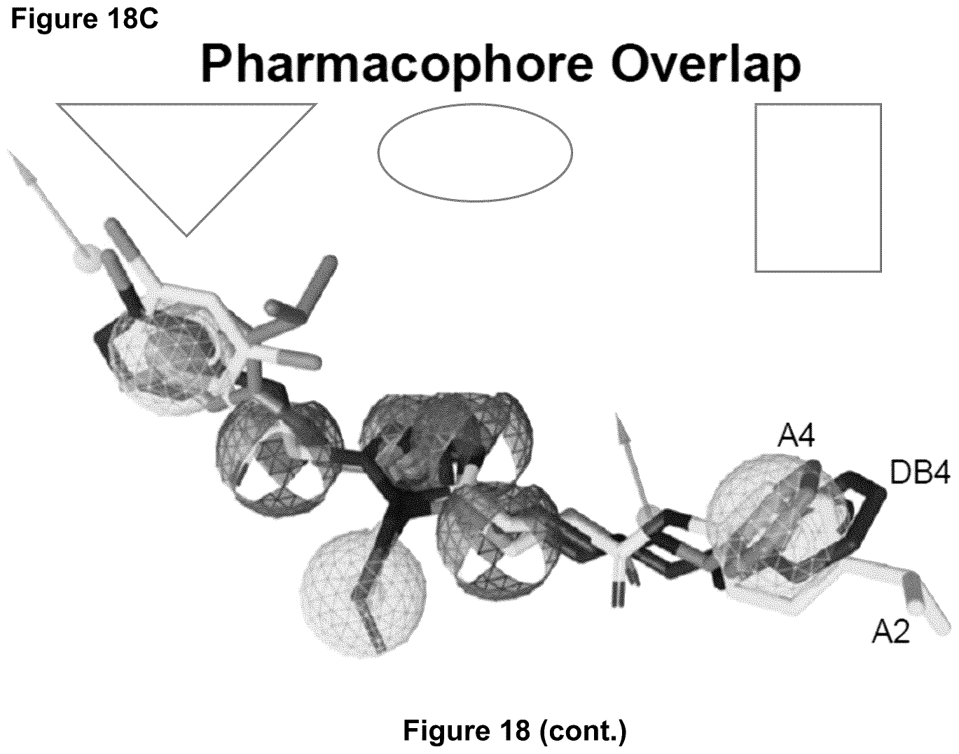

Molecular modeling (LigandScout) showed that hits of experimentally active RNR inhibitors contained a core hydrophilic 1,2,4-triazole pharmacophore (FIG. 18, ovals) flanked by two variable hydrophobic pharmacophores (FIG. 18, triangles and rectangles). The pharmacophoric features of these RNR inhibitors afford a basis for further structure modifications and the improvement of drug potency and PK/PD properties.

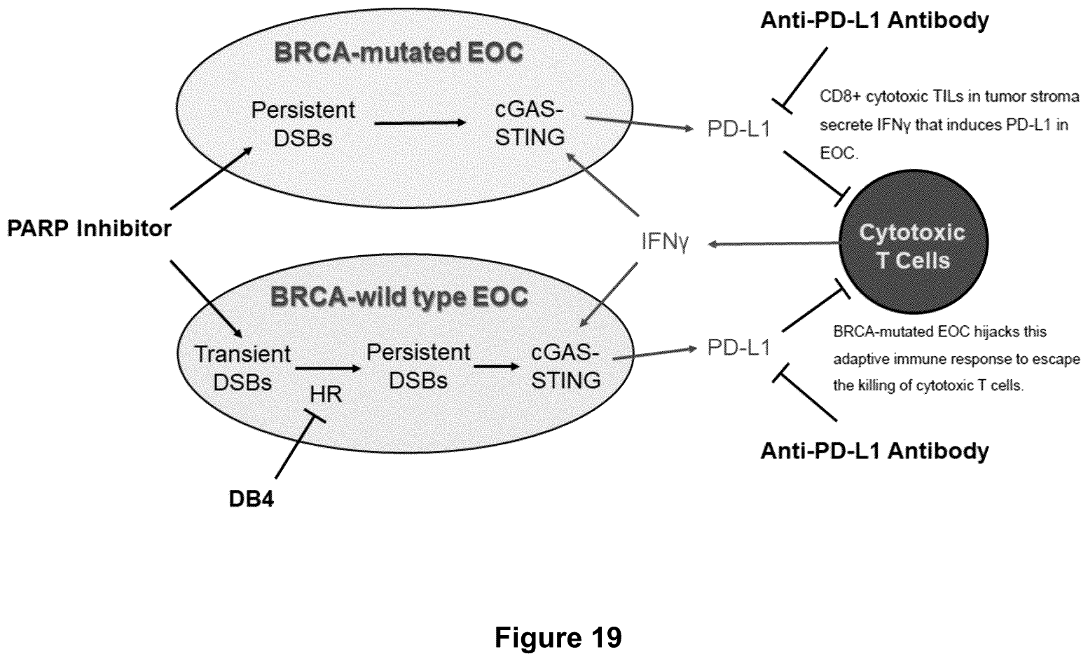

Furthermore, DB4 was shown to harness immune checkpoint blockage to treat BRCA-wild type EOC (FIG. 19). Specifically, BRCA-wild type EOC has low tumor neoantigens and exhibits poor immunogenicity (“cold” tumors). A lack of tumor infiltrating lymphocytes (TILs) in tumor stroma is a major hurdle to mount a strong immune attack on BRCA-wild type EOC using immune checkpoint blockage therapy. Thus, DB4 exploited the HR repair deficiency of BRCA-mutated EOC and enabled immune checkpoint inhibitors (e.g. an anti-PD-L1 antibody) to treat BRCA-wild type EOC.

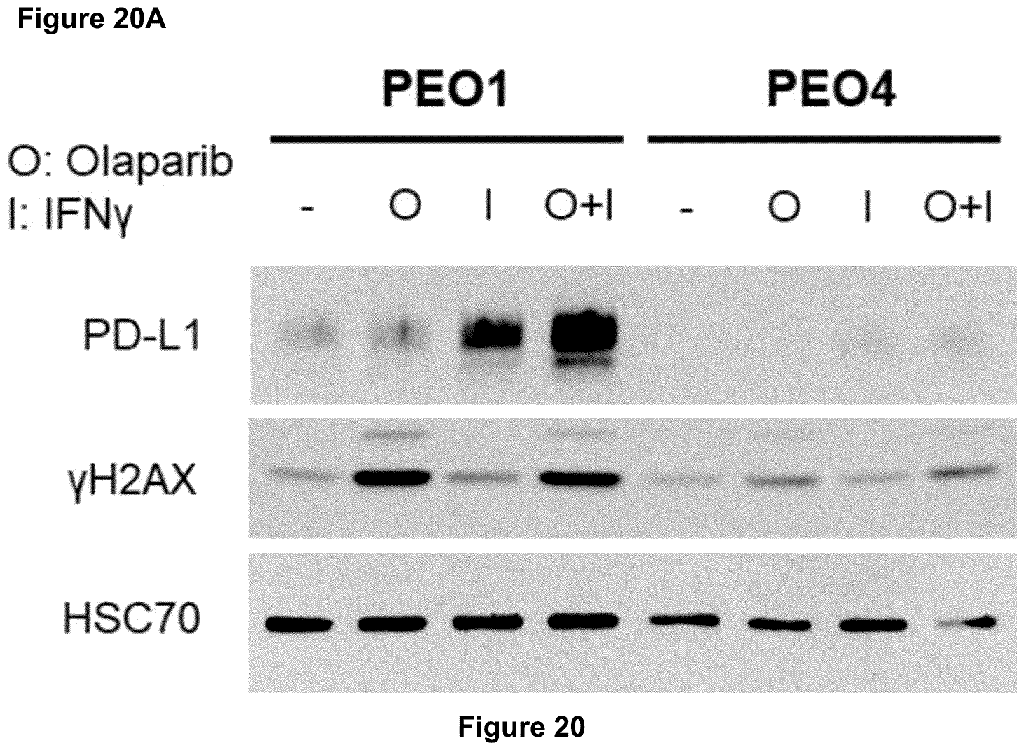

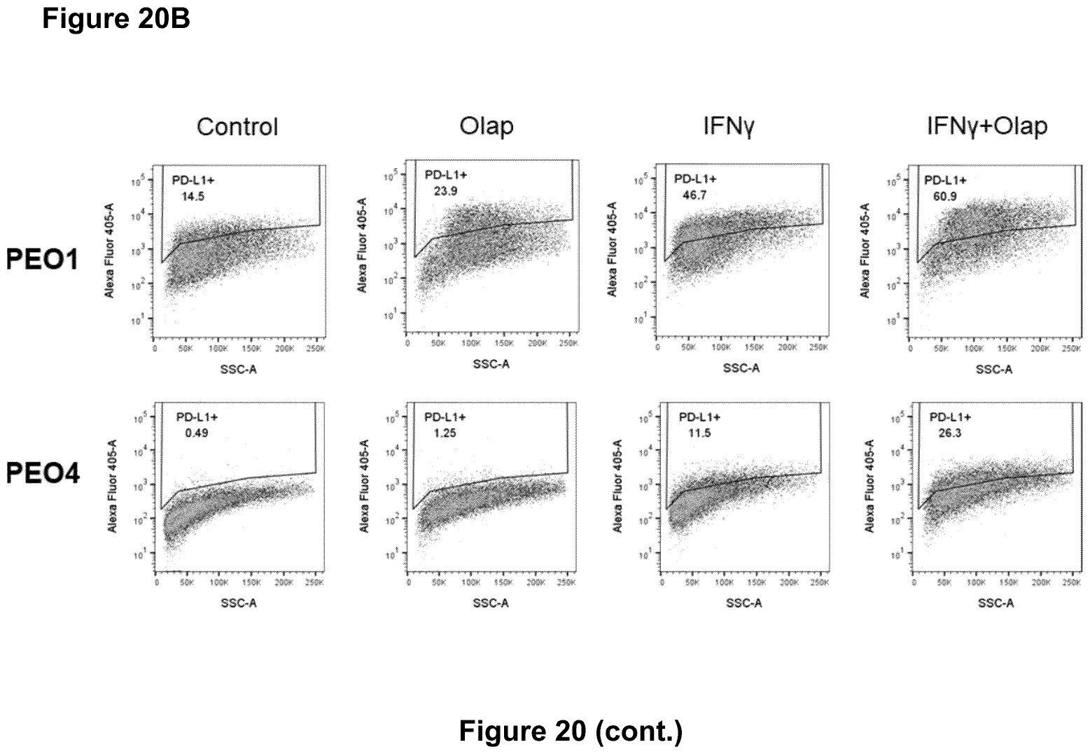

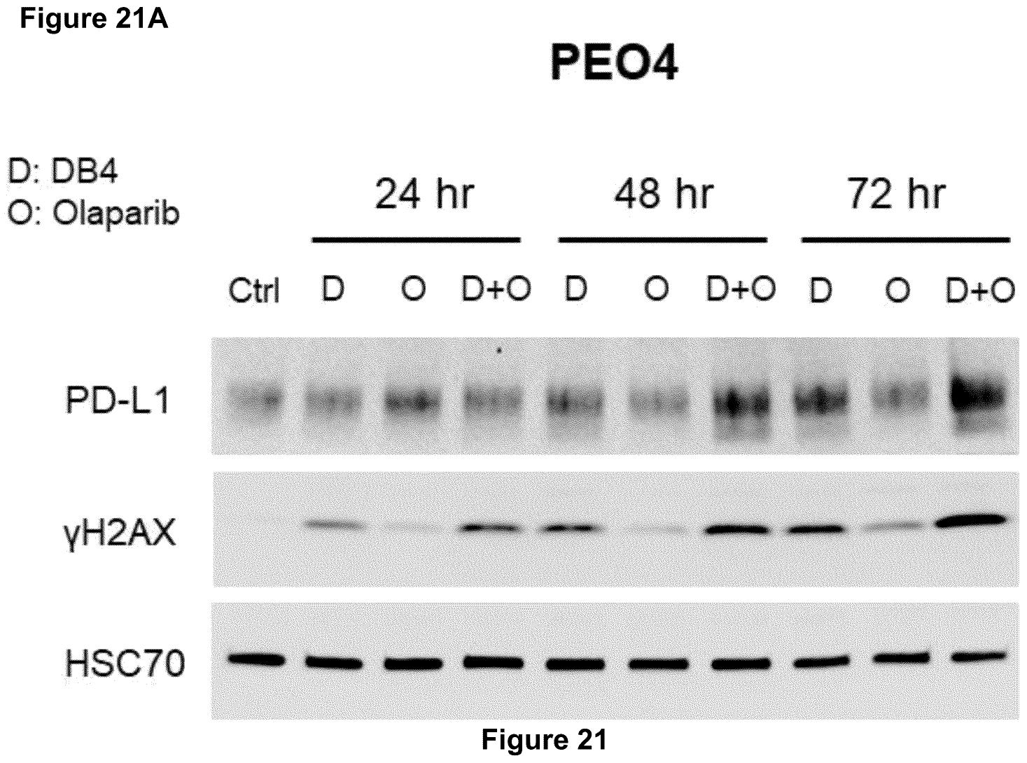

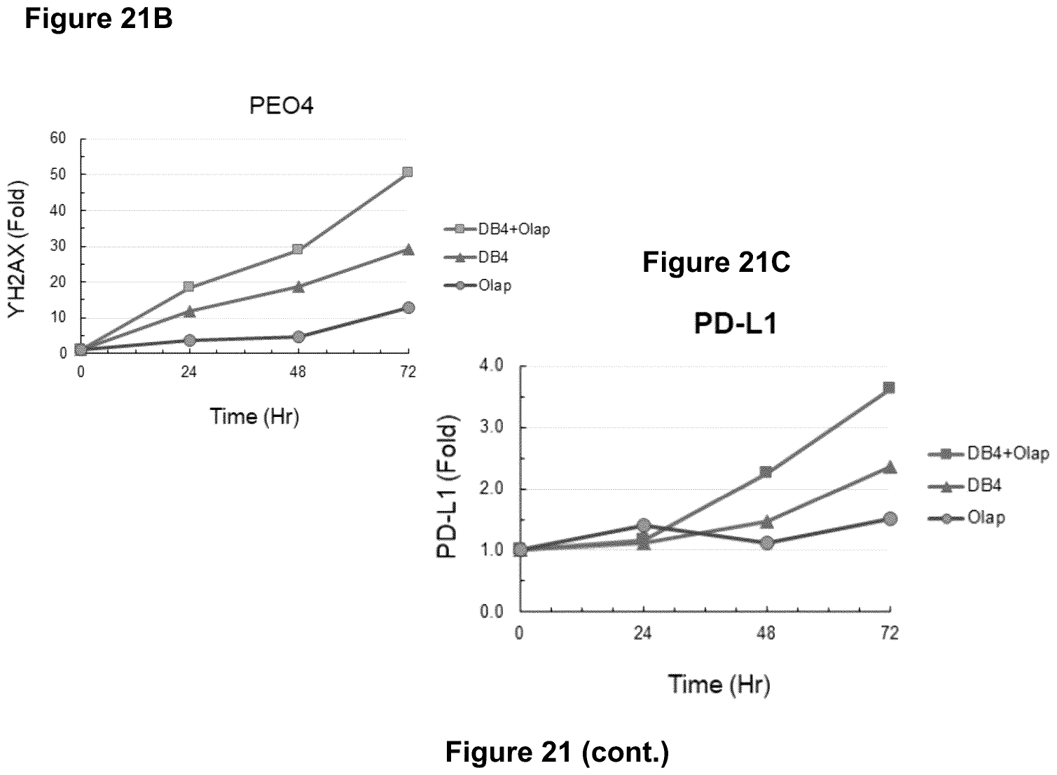

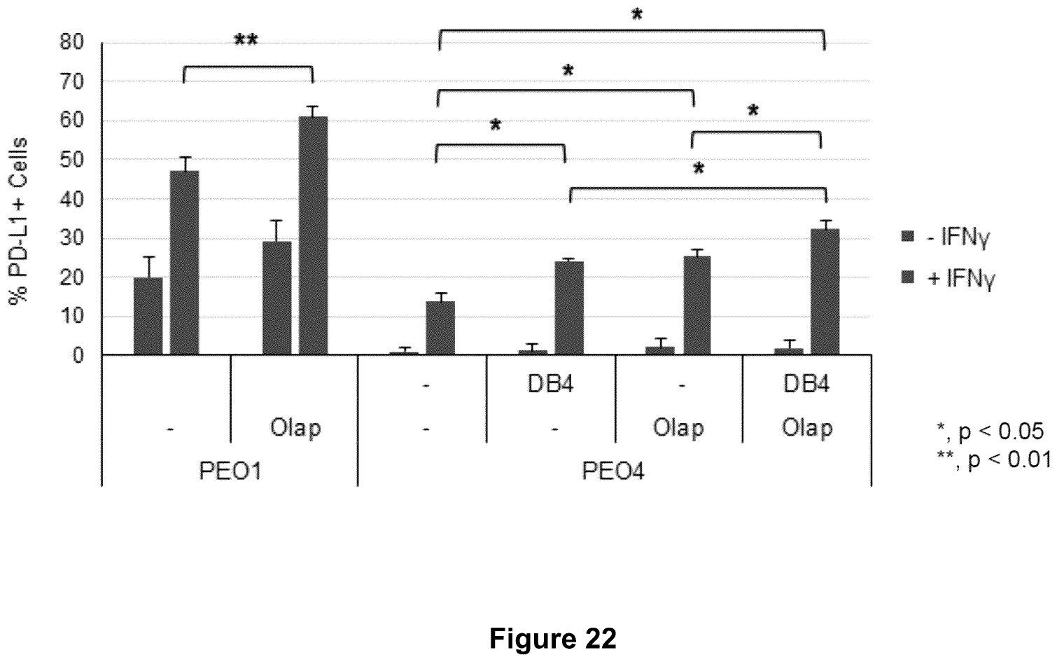

BRCA-mutated PEO1 cells were demonstrated to exhibit an increase in basal and olaparib-induced PD-L1 compared with BRCA-wild type PEO4 cells, and IFNγ markedly augmented the levels of basal and olaparib-induced PD-L1 in PEO1 cells while having relatively minor effects on that of PEO4 cells (FIG. 20). By blocking HR repair, DB4 enhanced olaparib-induced DSBs (γH2AX) and subsequently led to up-regulation of PD-L1 protein in BRCA-wild type EOC cells (FIG. 21). DB4 also significantly increased the PD-L1-positive population of PEO4 cells in the presence of IFNγ (FIG. 22). Furthermore, DB4 and olaparib synergistically increased the PD-L1-positive population of PEO4 cells in the presence of IFNγ.

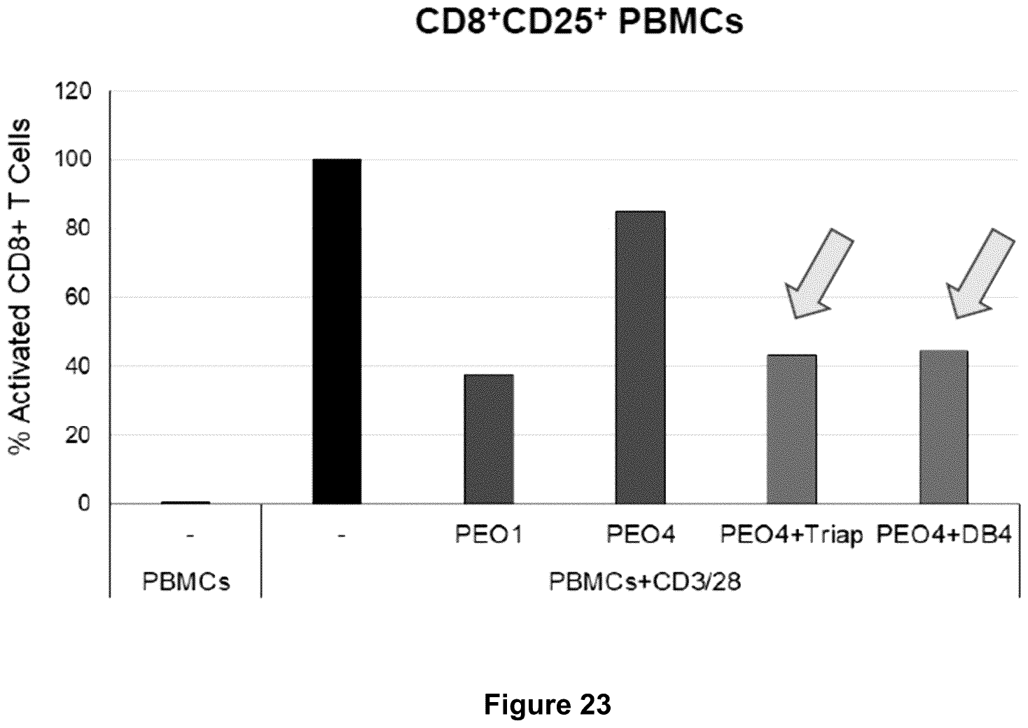

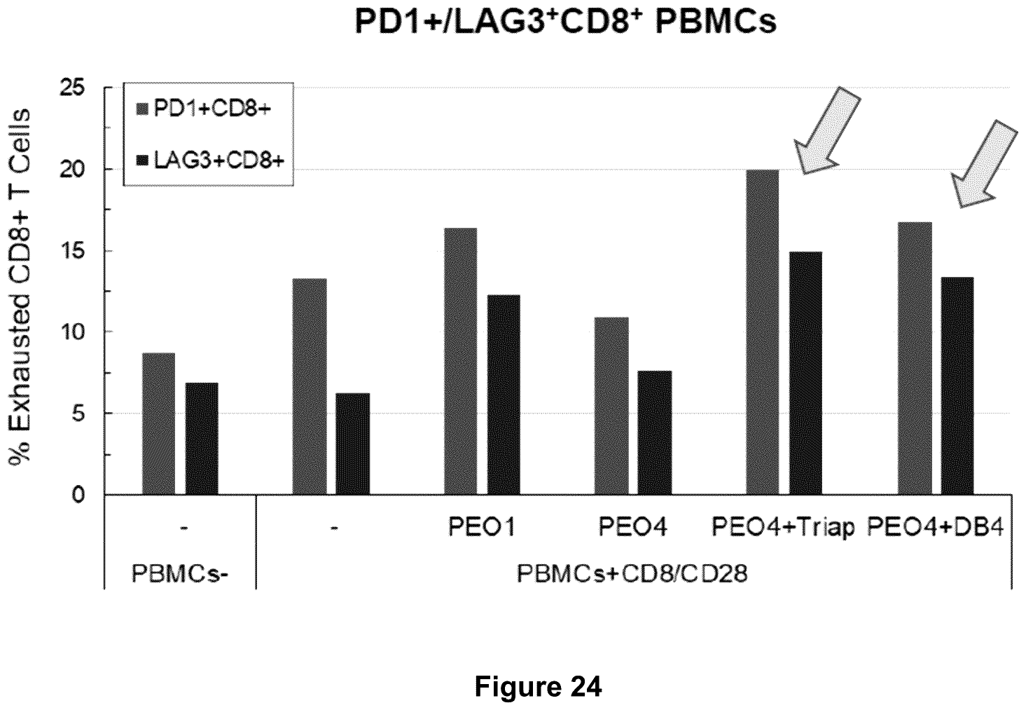

Further experiments were conducted using PBMCs containing T cells that were co-cultured with PEO1 or PEO4 cells and activated by CD3-CD28 beads (FIG. 23). PEO1 cells significantly suppressed the activation of CD8+ positive T cells due to expressing a high level of the PD-L1 compared with PEO4 cells (FIG. 24). DB4/triapine-treated PEO4 cells mimicked PEO1 cells as evidenced by suppressing T cell activation and enhancing T cell exhaustion (arrows). Both DB4 and triapine are RNR inhibitors. As such, although not bound by any particular theory, it was hypothesized that DB4 turned BRCA-wild type EOC into immunogenic “hot” tumors. DB4 promoted an increase in tumor neoantigens and the recruitment of tumor infiltrating lymphocytes (TILs) to tumor stroma, which enabled an effective antitumor response elicited by immune checkpoint inhibitors.

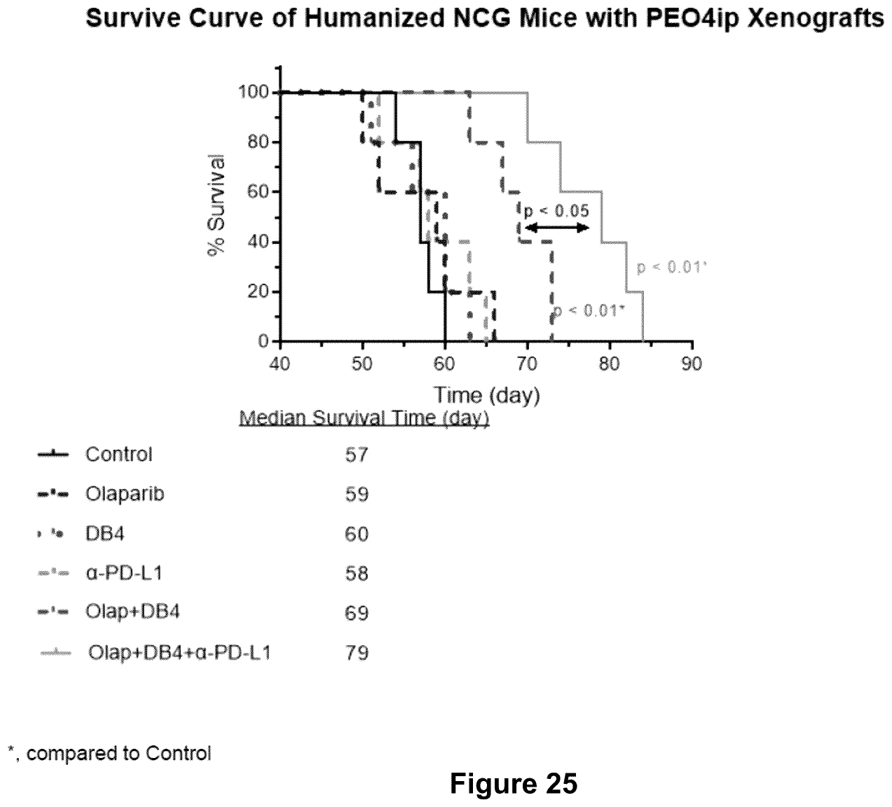

Studies investigating the survival of humanized NCG mice with PEO4ip xenografts were conducted. 1×10⁷ PEO4ip cells were implanted i.p. into NCG mice (N=5) at day 0. i.p. olaparib (50 mg/kg) was administered daily and DB4 (10 mg/kg) was administered every 2 days for 6 weeks (day 14-56). At day 27, 5×10⁶ human PBMCs were implanted i.p. into NCG mice (N=5) followed by i.p. anti-PD-L1 antibody (10 mg/kg) weekly administration for 4 weeks (day 28-56). As shown in FIG. 25, representative results demonstrated that the anti-PD-L1 antibody augmented the efficacy of the DB4-olaparib combination to suppress the progression of BRCA-wild type EOC xenografts in humanized mice.

In summary, DB4 disrupted HR repair and increased mutational burden or tumor neoantigens, thereby enhancing immunogenicity for immune checkpoint blockade therapy. DB4 was also demonstrated to up-regulate PD-L1 in BRCA-wild type EOC cells, which attenuated T cell activation and enhanced T cell exhaustion. Thus, the present studies demonstrated that PD-L1 blockade enhanced the combination of DB4 and olaparib to suppress the progression of BRCA-wild type EOC xenografts in humanized mice.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound represented by one of Formulae (I) to (V), or a racemate, enantiomer, diastereomer, pharmaceutically acceptable salt, solvate, hydrate, or derivative thereof: 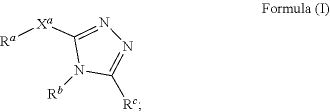
wherein in Formula (I):
X^{a} is selected from the group consisting of O, S, —SO₂, —N(R^{x}), —C(R^{x})(R^{y}), and —C═R^{z};
R^{a}, R^{b}, R^{c}, R^{x}, and R^{y} are each independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, and any combination thereof, wherein R^{a}, R^{b}, R^{c}, R^{x}, and R^{y} are each independently optionally substituted; and
R^{z} is selected from the group consisting of O, S, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxy heteroaryl, sulfoxyalkyl-heteroaryl, and any combination thereof, wherein R^{z} is optionally substituted: 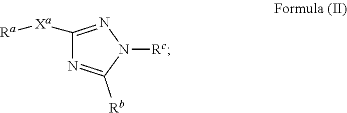
wherein in Formula (II):
X^{a} is selected from the group consisting of O, S, —SO₂, —N(R^{x}), —C(R^{x})(R^{y}), and —C═R^{z}:
R^{a}, R^{b}, R^{c}, R^{x}, and R^{y} are each independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxy cycloalkyl, sulfoxyalkenyl, sulfoxyalkynyl, sulfoxyaryl, sulfoxyalkyl-aryl, sulfoxyheteroaryl, sulfoxyalkyl-heteroaryl, and any combination thereof, wherein R^{a}, R^{b}, R^{c}, R^{x}, and R^{y} are each independently optionally substituted; and
R^{z} is selected from the group consisting of O, S, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxy cycloalkyl, sulfoxyalkenyl, sulfoxyalkynyl, sulfoxyaryl, sulfoxyalkyl-aryl, sulfoxyheteroaryl, sulfoxyalkyl-heteroaryl, and any combination thereof, wherein R^{z} is optionally substituted: 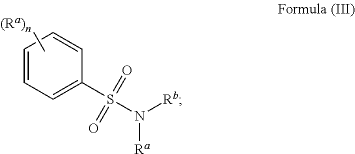
wherein in Formula (III):
each occurrence of R^{a}, R^{b}, and R^{c} is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, —NO₂, —CN, thiol, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, and any combination thereof, wherein R^{a}, R^{b}, and R^{c} are each independently optionally substituted; and wherein R^{b} and R^{c} are optionally joint to form a cycle;
n is an integer represented by 0 to 5: 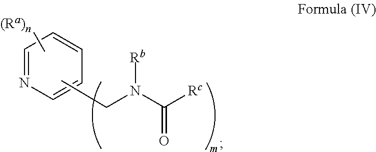
wherein in Formula (IV):
each occurrence of R^{a}, R^{b}, and R^{c} is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, —NO₂, —CN, thiol, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, and any combination thereof, wherein R^{a}, R^{b}, and R^{c} are each independently optionally substituted; and
m and n are each independently an integer represented by 0 to 5; and 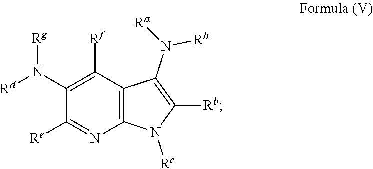
wherein in Formula (V):
R^{a}, R^{b}, R^{c}, R^{d}, R^{e}, R^{f}, R^{g}, and R^{h} are each independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, —NO₂, —CN, thiol, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, and any combination thereof, wherein R^{a}, R^{b}, R^{c}, R^{d}, R^{e}, R^{f}, R^{g}, and R^{h} are each independently optionally substituted.

2. The compound of claim 1, wherein in Formula (I):
R^{a}—X^{a} is selected from the group consisting of 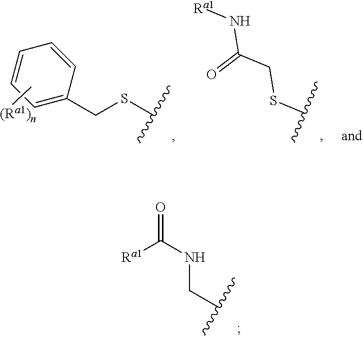
R^{b} is selected from the group consisting of, 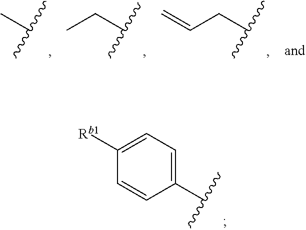
R^{c} is selected from the group consisting of 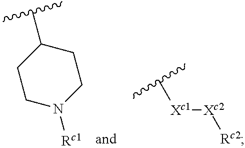
wherein each occurrence of R^{a1} is independently selected from the group consisting of hydrogen, deuterium, fluoro, chloro, bromo, iodo, alkyl, aryl, aryl alkyl, heteroaryl, heteroaryl alkyl, aminoaryl, aminoaryl alkyl, aminoheteroaryl, aminoheteroaryl alkyl, thioaryl, thioaryl alkyl, thioheteroaryl, thioheteroaryl alkyl, and any combination thereof,
R^{b1} is selected from the group consisting of hydrogen, deuterium, fluoro, chloro, bromo, iodo, alkyl, and any combination thereof,
R^{c1} and R^{c2} are independently selected from the group consisting of hydrogen, deuterium, fluoro, chloro, bromo, iodo, alkyl, aryl, aryl alkyl, heteroaryl, heteroaryl alkyl, alkoxycarbonyl, acyl, hydroxyl, hydroxyalkyl, hydroxyaryl, hydroxyalkyl-aryl, hydroxyheteroaryl, hydroxyalkyl-heteroaryl, alkoxy, carboxyl, carboxylate, ester, aminoaryl, aminoaryl alkyl, aminoheteroaryl, aminoheteroaryl alkyl, thioaryl, thioaryl alkyl, thioheteroaryl, thioheteroaryl alkyl, and any combination thereof;
X^{c1} and X^{c2} are independently selected from the group consisting of O, S, —SO₂, —N(R^{x}), and —C(R^{x})(R^{y});
wherein R^{x} and R^{y} are independently selected from the group consisting of hydrogen and alkyl;
R^{a1}, R^{b1}, R^{c1}, R^{c2}, R^{x}, and R^{y} are each independently optionally substituted; and
n is an integer represented by 0 to 5.

3. The compound of claim 1, wherein the compound represented by Formula (I) is selected from the group consisting of 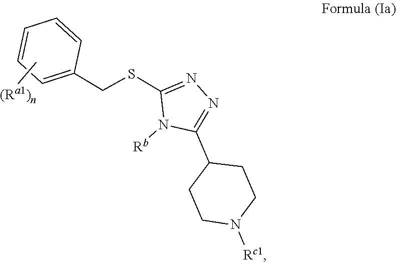
or a racemate, enantiomer, diastereomer, pharmaceutically acceptable salt, solvate, hydrate, or derivative thereof; 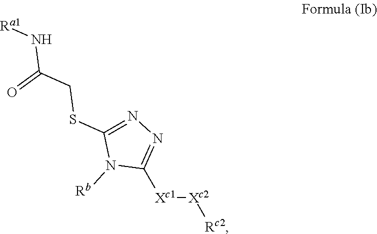
or a racemate, enantiomer, diastereomer, pharmaceutically acceptable salt, solvate, hydrate, or derivative thereof; and 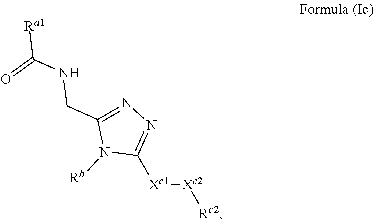
or a racemate, enantiomer, diastereomer, pharmaceutically acceptable salt, solvate, hydrate, or derivative thereof;
wherein each occurrence of R^{a1} is independently selected from the group consisting of hydrogen, deuterium, fluoro, chloro, bromo, iodo, alkyl, aryl, aryl alkyl, heteroaryl, heteroaryl alkyl, aminoaryl, aminoaryl alkyl, aminoheteroaryl, aminoheteroaryl alkyl, thioaryl, thioaryl alkyl, thioheteroaryl, thioheteroaryl alkyl, and any combination thereof,
R^{b1} is selected from the group consisting of hydrogen, deuterium, fluoro, chloro, bromo, iodo, alkyl, and any combination thereof,
R^{c1} and R^{c2} are independently selected from the group consisting of hydrogen, deuterium, fluoro, chloro, bromo, iodo, alkyl, aryl, aryl alkyl, heteroaryl, heteroaryl alkyl, alkoxycarbonyl, acyl, hydroxyl, hydroxyalkyl, hydroxyaryl, hydroxyalkyl-aryl, hydroxyheteroaryl, hydroxyalkyl-heteroaryl, alkoxy, carboxyl, carboxylate, ester, aminoaryl, aminoaryl alkyl, aminoheteroaryl, aminoheteroaryl alkyl, thioaryl, thioaryl alkyl, thioheteroaryl, thioheteroaryl alkyl, and any combination thereof;
X^{c1} and X^{c2} are independently selected from the group consisting of O, S, —SO₂, —N(R^{x}), and —C(R^{x})(R^{y});
wherein R^{x} and R^{y} are independently selected from the group consisting of hydrogen and alkyl;
R^{a1}, R^{b1}, R^{c1}, R^{c2}, R^{x}, and R^{y} are each independently optionally substituted; and
n is an integer represented by 0 to 5.

4. The compound of claim 3, wherein R^{c1} is selected from the group consisting of hydrogen, methyl, ethyl,

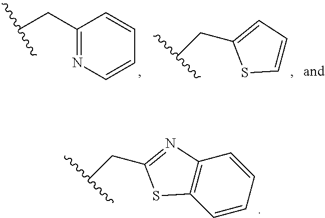

5. The compound of claim 3, wherein the compound represented by Formula (Ia) is selected from the group consisting of:

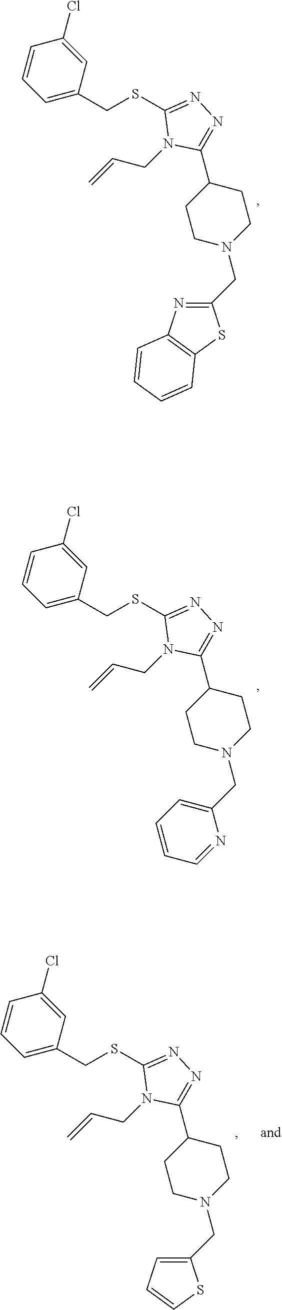 [SMILES: C=CCn1c(SCc2cccc(Cl)c2)nnc1C1CCN(Cc2nc3ccccc3s2)CC1 ; C=CCn1c(SCc2cccc(Cl)c2)nnc1C1CCN(Cc2ccccn2)CC1 ; C=CCn1c(SCc2cccc(Cl)c2)nnc1C1CCN(Cc2cccs2)CC1]

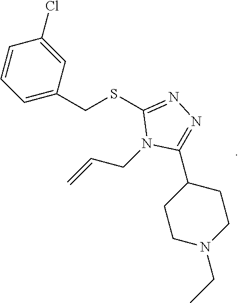 [SMILES: C=CCn1c(SCc2cccc(Cl)c2)nnc1C1CCN(CC)CC1]

6. The compound of claim 3, wherein the compound represented by Formula (Ib) is selected from the group consisting of

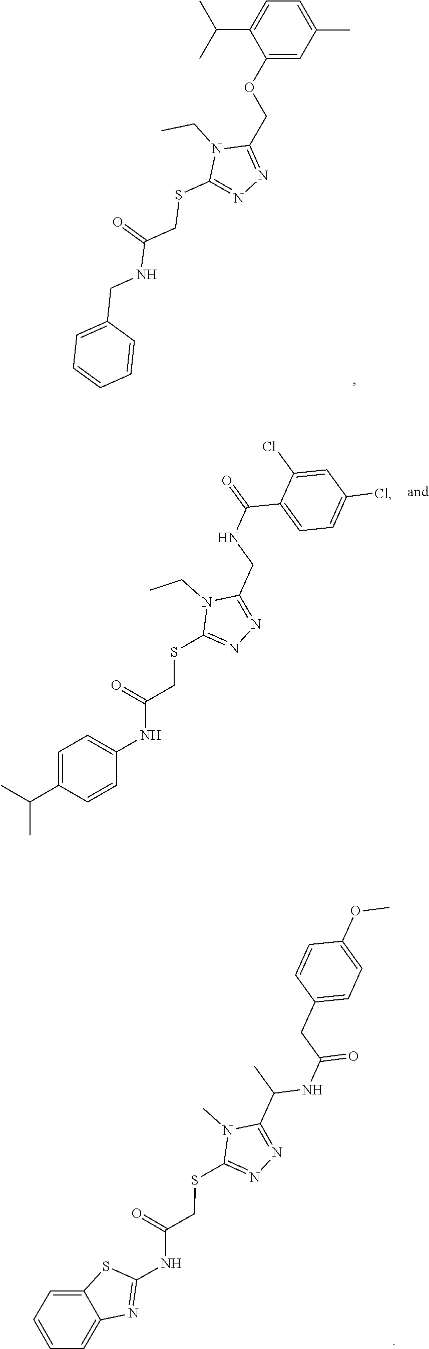

7. The compound of claim 3, wherein the compound represented by Formula (Ic) is

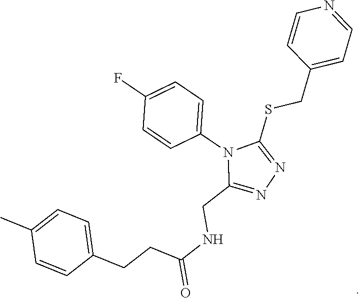 [SMILES: Cc1ccc(CCC(=O)NCc2nnc(SCc3ccncc3)n2-c2ccc(F)cc2)cc1]

8. The compound of claim 1, wherein in Formula (II):
each occurrence of R^{a}, R^{b}, and R^{c} is independently selected from the group consisting of hydrogen, deuterium, fluoro, chloro, bromo, iodo, alkyl, aryl, aryl alkyl, heteroaryl, heteroaryl alkyl, alkoxycarbonyl, amino, aminoalkyl, aminoalkyl-aryl, aminoalkyl-heteroaryl, amido, secondary amide, tertiary amide, aminoalkenyl, aminoalkynyl, aminoacetate, acyl, hydroxyl, hydroxyalkyl, hydroxyaryl, hydroxyalkyl-aryl, hydroxyheteroaryl, hydroxyalkyl-heteroaryl, alkoxy, carboxyl, carboxylate, ester, aminoaryl, aminoaryl alkyl, aminoheteroaryl, aminoheteroaryl alkyl, thioaryl, thioaryl alkyl, thioheteroaryl, thioheteroaryl alkyl, and any combination thereof,
wherein R^{a}, R^{b}, and R^{c} are each independently optionally substituted;
R^{b} and R^{c} are optionally joint to form a cycle; and
n is an integer represented by 0 to 5;

9. The compound of claim 1, wherein the compound represented by Formula (II) is selected from the group consisting of

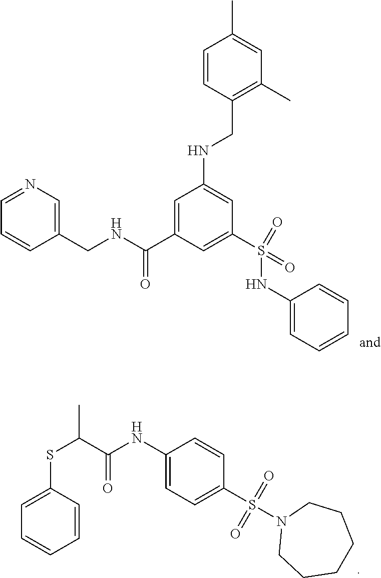 [SMILES: Cc1ccc(CNc2cc(C(=O)NCc3cccnc3)cc(S(=O)(=O)Nc3ccccc3)c2)c(C)c1 ; CC(Sc1ccccc1)C(=O)Nc1ccc(S(=O)(=O)N2CCCCCC2)cc1]

10. The compound of claim 1, wherein the compound represented by Formula (IV) is a compound represented by Formula (IVa) 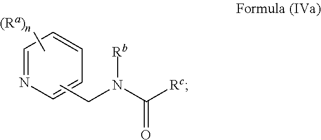
or a racemate, enantiomer, diastereomer, pharmaceutically acceptable salt, solvate, hydrate, or derivative thereof;
wherein each occurrence of R^{a}, R^{b}, and R^{c} is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminocycloalkyl, aminoaryl, aminoalkyl-aryl, aminoheteroaryl, aminoalkyl-heteroaryl, amido, secondary amide, tertiary amide, aminoalkenyl, aminoalkynyl, aminoacetate, acyl, hydroxyl, hydroxyalkyl, hydroxycycloalkyl, hydroxyalkenyl, hydroxyalkynyl, hydroxyaryl, hydroxyalkyl-aryl, hydroxyheteroaryl, hydroxyalkyl-heteroaryl, alkoxy, carboxyl, carboxylate, ester, —NO₂, —CN, thiol, thioalkyl, thiocycloalkyl, thioalkenyl, thioalkynyl, thioaryl, thioalkyl-aryl, thioheteroaryl, and thioalkyl-heteroaryl, sulfoxy, sulfoxyalkyl, sulfoxycycloalkyl, sulfoxyalkenyl, sulfoxyalkynyl, sulfoxyaryl, sulfoxyalkyl-aryl, sulfoxyheteroaryl, sulfoxyalkyl-heteroaryl, and any combination thereof, wherein R^{a}, R^{b}, and R^{c} are each independently optionally substituted; and
n is an integer represented by 0 to 5.

11. The compound of claim 1, wherein the compound is a ribonucleotide reductase (RNR) inhibitor.

12. A composition comprising at least one compound of claim 1.

13. The composition of claim 12, wherein the composition further comprises at least one poly ADP-ribose polymerase (PARP) inhibitor.

14. The composition of claim 13, wherein the poly ADP-ribose polymerase (PARP) inhibitor is olaparib.

15. A method of reducing the level or activity of poly ADP-ribose polymerase (PARP), wherein the method comprises administering a therapeutically effective amount of at least one compound of claim 1 or a composition thereof.

16. A method of reducing the level or activity of ribonucleotide reductase (RNR), wherein the method comprises administering a therapeutically effective amount of at least one compound of claim 1 or a composition thereof.

17. A method of reducing homologous recombination (HR) repair, wherein the method comprises administering a therapeutically effective amount of at least one compound of claim 1 or a composition thereof.

18. A method of reducing the level or activity of cyclin A2, wherein the method comprises administering a therapeutically effective amount of at least one compound of claim 1 or a composition thereof.

19. A method of reducing the level or activity of Rad51 foci, wherein the method comprises administering a therapeutically effective amount of at least one compound of claim 1 or a composition thereof.

20. A method of reducing the level or activity of poly ADP-ribose polymerase (PARP), ribonucleotide reductase (RNR), homologous recombination (HR) repair, cyclin A2, Rad51 foci, or any combination thereof, wherein the method comprises administering a therapeutically effective amount of at least one compound of claim 1 or a composition thereof.

21. A method of inducing at least one double strand break (DSB), wherein the method comprises administering a therapeutically effective amount of at least one compound of claim 1 or a composition thereof.

22. A method of regulating the level or activity of at least one selected from the group consisting of checkpoint kinase 1 (Chk1), ataxia telangiectasia and Rad3-related protein (ATR), replication protein A32 (RPA32), histone H2A, histone H2AX, histone H1, cyclin-dependent kinase 1 (CDK1), and cyclin-dependent kinase 2 (CDK2), wherein the method comprises administering a therapeutically effective amount of at least one compound of claim 1 or a composition thereof.

23. A method of regulating the phosphorylation of at least one selected from the group consisting of checkpoint kinase 1 (Chk1), ataxia telangiectasia and Rad3-related protein (ATR), replication protein A32 (RPA32), histone H2A, histone H2AX, histone H1, cyclin-dependent kinase 1 (CDK1), and cyclin-dependent kinase 2 (CDK2), wherein the method comprises administering a therapeutically effective amount of at least one compound of claim 1 or a composition thereof.

24. A method of increasing the level or activity of programmed death-ligand 1 (PD-L1), wherein the method comprises administering a therapeutically effective amount of at least one compound of claim 1 or a composition thereof.

25. A method of increasing the level or activity of at least one tumor neoantigen, wherein the method comprises administering a therapeutically effective amount of at least one compound of claim 1 or a composition thereof.

26. A method of preventing or treating cancer in a subject in need thereof, wherein the method comprises administering a therapeutically effective amount of at least one compound of claim 1 or a composition thereof to the subject.

27. The method of claim 26, wherein the cancer is ovarian cancer, BRCA-wild type cancer, or a combination thereof.

28. A method of administering at least one compound of claim 1 or a composition thereof to a subject, wherein the subject has cancer.

29. The method of claim 28, wherein the cancer is ovarian cancer, BRCA-wild type cancer, or a combination thereof.

30. The method of claim 28, wherein the method further comprises administering at least one poly ADP-ribose polymerase (PARP) inhibitor.

31. The method of claim 30, wherein the at least one PARP inhibitor is olaparib.

32. The method of claim 31, wherein the at least one PARP inhibitor is administered at the same time as the compound of claim 1 or a composition thereof.
